SEQUENCE LISTING (116 sequences)

SEQ ID NO: 1 - protein, 130 aa, Mus musculus
  mat_peptide (1)..(130): Galecctin-2
MSEKFEVKDLNMKPGMSLKIKGKIHNDVDRFLINLGQGKETLNLHFNPRFDESTIVCNTSEGGRWGQEQRENHMCFSPGSEVKITITFQDKDFKVTLPDGHQLTFPNRLGHNQLHYLSMGGLQISSFKLE

SEQ ID NO: 2 - protein, 2583 aa, Mus musculus
  mat_peptide (1)..(2583): IgGFc-binding protein (Fcgbp)
MGSPWKWWALWAGATLLWALLTPAEASCQGIQCASGQRCQMVSGKARCVAESTAVCRAQGDPHYTTFDGRRYDMMGTCSYTMAELCGSDETLPAFSVEAKNEHRGSRQVSYVGLVTVYAYSHSVSLVRGEIGFVRIDNQRSRLPASLSEGRLRVHKSGTRGVIEMDFGLVVTYDWDGQLTLSLPKRFQDQVCGLCGNYNGDPADDFLTPDLDQAPDALEFANSWKLDDGDYLCDDGCHNSCPSCTPGQTQHYKGDRLCGMLTLSTGPFSACHEFLDPKPFLDDCVFDLCVTGGERLSLCRSLSAYAQACVELGVTLENWRLPASCPMSCPANSCYDPCSPACPPSCNSEAVPTNCSSRPCVEGCVCLPGFVASGGDCVPVSSCGCIYQGRLLAPGQEVFDDDRCRRRCTCDGATQKVTCRDTTGCPSGERCNVQNGLLGCYPDNFASCQASGDPHYVSFDGKRFDFMGTCTYLLVGSCGQNAALPAFKVLVENEHRGSQTVSYTRAVRVVAHGVEVAVRRENPGRVLIDGVLQYLPFQAAGGKIQVYRQGNDAIVSIDFGLTVTYNWDAHVTAKVPSSYAKDVCGLCGNFNGNPDDDLALKGGGQASNVLDFGNSWQEEIIPGCGATEPGDCPQLDSLVTQQIQDKKECGILADPEGPFRECHKLLNPQGAIRDCVYDLCLLPGQSGPLCDALAAYAAACQAAGGTVHPWRSEELCPLTCPPNSHYEQCSYGCPLSCGDLPVQGGCGSECREGCVCNEGFALSGESCVPLASCGCVHEGAYHAPGETFYPGPGCDSLCHCEEGGLVSCEPSSCGPQEACQPSNGVLGCVAVGTTTCQASGDPHYVTFDGRRFDFMGTCVYVLAQTCGNRPGLHQFTVLQENEAWGNGKVSVTKVITVLVANYTLRLEQSQWKVKVNGVDTKLPVMLDGGKIRVSQHGSDVVIETDFGLRVAYDLVYYVRVTIPGNYYKQMCGLCGDYNGDPKDDFQKPDGSQTTDPSDFGNSWEEAVPDSPCAPVPPCTGDDCDTECSPELQDKYHGEQFCGLLTSPTGPLAACHKLLDPQGPLQDCVFDLCLGGGNQSILCNIIHAYVSACQAAGGQVEPWRTETFCPMECPPHSHYEVCADTCSLGCWALNTPQQCPEGCAEGCECDSGFLYNGKACVPIEQCGCYHNGVYYEPEESVLIENCQQHCVCQPGKGMMCQDHSCKPGQVCEPSGGVLTCVTKDPCHGITCRPQETCKVQGGEGVCVPNYNSTCWLWGDPHYNSFDGWSFDFQGTCNYLLAGTLCPGVNAEGLTPFTVTTKNENRGSPAVSYVRQVTVTTLNTNISIHKNEIGKVRVNGVLMALPVYLAGGRISVINGGSKAVLETDFGLQVTYDWNWRVDVTLPSSYHGAVCGLCGNMDKNHQNDQVFPNGTMAPSIPTWGGSWQVPGWDPLCWHECQGSCPTCPEDRVEEYEGPGFCGPLAPGTGSPFTSCHAHVPPESFFKGCVLDVCLGGGSKDILCQALAAYAAACQAAGIKIEDWRTQAGCEITCPDNSHYELCGPPCPASCPPPARHTAPTVCDGPCVEGCQCDEGFVLSADQCVPLDGGCGCWVNGTYYEAGTEFWADTTCSKRCHCGPGGDSLVCKPASCGLGEECALLPSGEIGCQPTSITECQAWGDPHYTTLDGHRFDFQGTCEYLLSAPCHEPPTGTEYFNVTVANEHRGSQAVSYTRSVTLQIYGLSLTLSAQWPRKLQVNGEFVALPFHLDQKLSVYISGADVVVNTASGVSLAFDGDSFVRLRVPAAYAGTLCGLCGNYNKNPNDDLTAVGGKPEGWKVGGAPGCDQCEPEPCPKPCTPEEQEPFRGPDACGIITAPEGPLAPCHSLVPPTQYFEACLLDACQVQGHPGGLCPAIATYVAACQAAGAQLGEWRKPDFCPLQCPAHSHYQLCGDSCPVSCPSLSAPVGCETICREGCVCDAGFVLSGDTCVPVGQCGCLYQGRYYVLGATFYPGPECERLCECGPDGQVTCQEGADCEPYEECRIENGVQACHPTGCGHCLANGGLHYVTLDGRVYDLHGSCSYVLASVCHPKPGDEEFSIVLEKNSAGDPQRVVVTVAGQVVGLARGPQVTVDGEVVTLPVATGHVSVTAEGRNIVLQTNKGMKVLFDGDAHILMSIPSSFRGRLCGLCGNFNGNWSDDFVLPSGAVAPNVEAFGTAWRAPGSSLGCGEGCGPQGCPVCLAEETQAYEKNDACGKIRDPHGPFAACHKVLSPLEYFRQCVYDMCAHKGDKAYLCRSLAAYTAACQAAGAAVKPWRTDSVCPLQCPAHSHYSICTRSCQGSCAALSGLTGCTTRCFEGCECDDHFLLSHGVCIPAQDCGCVHNGQYMPVNSSLMSSDCSERCFCSPNNGLTCHEAGCPSGHVCEIQAGVRECQAARGLCSISVGANLTTFDGAHNAISSPGVYELSSRCPGLQKNVPWYRVLADVQPCHNNDKIVSKVHIFFQDGLVTVIPSKGAWVNGLRVDLPATVLTSVSVRRMPDGSMLVHQKAGVTVWLGKDGLLDVMVGDDLAAMLCGACGNFDGDQTNDAYGSQGKTPIEKWRAQDFSPCSN

SEQ ID NO: 3 - protein, 326 aa, Mus musculus
  mat_peptide (1)..(326): galectin-4
MAYVPAPGYQPTYNPTLPYKRPIPGGLSVGMSVYIQGMAKENMRRFHVNFAVGQDDGADVAFHFNPRFDGWDKVVFNTMQSGQWGKEEKKKSMPFQKGKHFELVFMVMPEHYKVVVNGNSFYEYGHRLPVQMVTHLQVDGDLELQSINFLGGQPAAAPYPGAMTIPAYPAGSPGYNPPQMNTLPVMTGPPVFNPRVPYVGALQGGLTVRRTIIIKGYVLPTARNFVINFKVGSSGDIALHLNPRIGDSVVRNSFMNGSWGAEERKVAYNPFGPGQFFDLSIRCGMDRFKVFANGQHLFDFSHRFQAFQMVDTLEINGDITLSYVQI

SEQ ID NO: 4 - protein, 2680 aa, Mus musculus
  mat_peptide (1)..(2680): mucin-2 (Fragments)
MGLPLARLVAACLVLALAKGSELQKEARSRNHVCSTWGDFHYKTFDGDVYRFPGLCDYNFASDCRDSYKEFAVHLKRGLGEAGGHSQIESILITIKDDTIYLTHKLAVVNGAMVSTPHYSSGLLIEKNDAYTKVYSRAGLSLMWNREDALMVELDSRFQNHTCGLCGDFNGMQTNYEFLSEEGIQFSAIEFGNMQKINKPEVQCEDPEAVQEPESCSEHRAECERLLTSAAFEDCQTRVPVESYVRACMHDRCQCPKGGACECSTLAEFSRQCSHAGGRPENWRTASLCPKKCPNNMVYLESSSPCVDTCSHLEVSSLCEEHYMDGCFCPEGTVYDDITGSGCIPVSQCHCKLHGHLYMPGQEFTNDCEQCVCNAGRWVCKDLPCPETCALEGGSHITTFDGKKFTFHGDCYYVLTKSEHNDSYALLGELASCGSTDKQTCLKTVVLLTDDKKNVVAFKSGGSVLLNEMEVTLPHVAASFSIFQPSSYHIVVNTKFGLRLQIQLLPVMQLFVTLDQAAQGQVQGLCGNFNGLESDDFMTSGGMVEATGAGFANTWKAQSSCHDKLDWLDDPCSLNIETNYAEHWCSLLKRSETPFARCHLAVDPTEYYKRCKYDTCNCQNNEDCMCAALSSYARACAAKGVMLWGWRERVCNKDVHACPSSQIFMYNLTTCQQTCRSLSEGDSHCLKGFAPVEGCGCPDHTFMDEKGRCVPLAKCSCYHHGLYLEAGDVILRQEERCICRNGRLQCTQVKLIGHTCQYPKILVDCNNLTALAVRKPRPTSCQTLVAGYYHTECISGCVCPDGLLDDGRGGCVEEDKCPCIHNKDLYSSGESIKLDCNNTCTCQKGRWECTRYACHSTCSIYGSGHYITFDGKHYDFDGHCSYVAVQDYCGQNSTGSFSIITENVPCGTTGVTCSKAIKIFIGGTELKLVDKHRVVKQLEEGHHVPYITREVGQYLVVEASSGIIVIWDKKTTIFIKLDPSYKGTVCGLCGNFDDQTKNDFTTRDHMVVTSELDFGNSWKEASTCPDVSHNPDPCSLNPHRRSWAEKQCSIIKSRVFKVCHSKVDPTVFYEACVHDSCSCDTGGDCDCFCSAVASYAQECTKAEACVFWRTPDLCPIFCDYYNPPDECEWHYEPCGNRSFETCRTLNGIHSNISVSYLEGCYPRCPEDRPIYDEDLKKCVTGDKCGCYIEDTRYPPGGSVPTDEICKSCTCTNTSKIECHPDEGKILNMTQDGIFCYWEFCGPNGTVGQHFNICGSSTAIPSTTTSFTTISTPISTTPISTTITTTTVTMTTEQVPCCFWSDWINKYHPTKENGGDRETFTHVCSAPEDIECRAATDPKLSWEELGQKVQCNVSTGLICNNEDQYGIGEFELCYDYEIRVNCCYPMEYCTPSTISPTTSTTTLSTTPPTSSPTTLPTSSPVTSSATLPTTSSITSTISPTTSPSTATQTISVTTSQTSSSATPPNSSPTSSATTSPTTSSGTSTATSPSTSPTTSSTFTTPPSTTCIDDCKWTGWLDSGKPTYDIKSGDFELIKGVCEPHWEVQNISCRAVMHSNIPLDQLGQIVVCNKEVGLVCKNEDQEIGGIIPMRMCLNYEINVYCCNPICFTSTPSSTTTETPTTTSTTKTSILTSTTTQTPSPSPTTTVTPTPAPTTTQIPTSTSTTTQTTTPTPITETSTPTSTISQTPSPASTTTVTPATTSTTTETSTSTSTTTQTTSPTPTVTETSTPRSTTTQTPSPVPTTTVTSTPTPTIGETTTPKRPPSTSTPTSFTVPTETTTQTRPLSTTPTTLETTRTSSWGTFSSTSPITSPSTVWTHTETQVTCCVLNEMFYGPGELVYNSTHGGTCFYVNCSLDCHLQFFNWSCPSTPSTPTPSTPTPTPSQTTTPSTTSSKSTPSTPQSTSPKSTLSTPTKTTPYGCPDFDPPRQVNETWWLCNCTMAICNHDNVVEIVPLKCDPPPMPTCANGLKPVRVPDADNCCWHWECDCYCTGWGDPHFVTFDGLYYSYQGNCTYVLVEEITPTVDNFGVYIDNYHCDANDKVSCPRTLIVRHETQEVQIKTVRMMPIEVEVQVNKQLVALPYKKYGLEVYESGINIVVNISRLEAKISYNGLSFSIRLPYKLFVNNTKGQCGTCTNNTADDCILPSGKIISDCEIAADEWLVNDPSKPHCPHKGLTTKRPATTTPGLSLNNCTVSPVCHLIMDSLFSQCHAFVPPKHYYEACLFDSCYVPGSNMECASVQAYATLCAKEGVCIDWRNHTQGVCSVKCPPHKQYQACGPEEEPTCQPSSSQNSTLLVEGCFCPEGTTKFAPGYDVCVKTCGCVGPDNVPREFGEHFEFDCKDCVCREGGSGIVCQPKKCSGGNQTTCEEDGTYLVVETNPDDKCCNITSCKCDTKRCKAERPTCLLGFEVKTEIVPGKCCPVYSCVPKGVCVHQNAEYQPGSPVYSNKCQDCVCTNILDNSTQLNVISCTHVPCNISCSSGFELVDVPGECCKKCQQTHCIIEGPKQQYIILKPGEIHKNPSNKCTFFSCMKINNQLISSVSNITCPDFNPSDCVSGSITYMPNGCCKTCIPQNQTRVPCSAVSVMKEISYNGCTKNISMNYCFGSCGTFAMYSAQVQGLDHRCSCCKEEKTSVRSVTLECPDGSELSHTYTHIESCLCQDTVCGLPQAQQVRTRRSSPRFLGRK

SEQ ID NO: 5 - protein, 175 aa, Mus musculus
  mat_peptide (1)..(175): anterior gradient protein 2 homolog
MEKFSVSAILLLVAISGTLAKDTTVKSGAKKDPKDSRPKLPQTLSRGWGDQLIWTQTYEEALYRSKTSNRPLMVIHHLDECPHSQALKKVFAEHKEIQKLAEQFVLLNLVYETTDKHLSPDGQYVPRIVFVDPSLTVRADITGRYSNRLYAYEPSDTALLYDNMKKALKLLKTEL

SEQ ID NO: 6 - protein, 827 aa, Mus musculus
  mat_peptide (1)..(827): cadherin-17
MVSAQLHFLCLLTLYLTCGYGEEGKFSGPLKPMTFSIFEGQEPSQVIFQFKTNPPAVTFELTGETDGIFKIEKDGLLYHTRALDRETRAVHHLQLAALDSHGAIVDGPVPITIEVKDINDNRPTFLQSKYEGSVRQNSRPGKPFMYVNATDLDDPATPNGQLFYQIVIQLPQINDVMYFQIDSKTGAISLTPEGSQELDPVKNPSYNLVVSVKDMGGQSENSFSDTTYVDISIRENIWKAPEPVEIRENSTDPHPIKITQVQWNDPGAQYSLVNKEKLSPFPFSIDQEGNIYVTQALDREEKNSHVFFATAKDENGKPLAYPLEIYVKVIDINDNPPTCLSPVTVFEVQENEPLGNSIGIFEAHDMDEANNINSILKYKLVDQTPKVPSDGLFLIGEYEGKVQLSKQSLKKQDSPQYNLSIEVSDVDFKTLCYIQVNVIDINDQIPIFETSNYGSKTLSEDTAIGSTILIIQATDADEPFTGSSKILYKIVQGDTEGRLEVVTDPTTNAGYVKIKKPLDFETQPVSSIVFQAENPEPLVKGIEYNASSFASFELIVTDVNEVPVFPQRIFQANVSEDAAVGSRVGNVTARDPEGLTVSYSLKGNMRGWLKIDSVTGEIFSAAPLDRETESVYRVQVVATEVGGSSLSSTADFHLVLTDVNDNPPRLAKDYTGLFFCHPLSAPGSLIFEVTDDDQQSLRRPKFTFALGREGLQSDWEVSKINGTHARLSTRHTRFEEQVYNIPIRINDGGQPPMEGTVFLPVTFCQCVEGSCFRPAGRQDGIPTVGMAVGILLTTFLVIGIILAVVFIRMRKDKVENPQSPENKPLRS

SEQ ID NO: 7 - protein, 167 aa, Mus musculus
  mat_peptide (1)..(167): zymogen granule membrane protein 16
MLAVALLVLLCASASANSIQSRTSSYSGEYGGKGGKRFSHSGNQLDGPITAFRIRVNRYYIVGLQVRYGTVWSDYVGGTQGDLEEIFLHPGESVIQVSGKYKSYVKQMIFVTDKGRYLPFGKASGTSFNAVPLHPNTVLRFISGRSGSAIDSISLHWDTYPSHCNTC

SEQ ID NO: 8 - protein, 771 aa, Mus musculus
  mat_peptide (1)..(771): metal transporter CNNM4
MAPGGGGGRRDGWPARGRLLLAALLLLWTRAASGQSSPQQSVILGMRLASCNKSCGMNPDGIIFVSEGSTVNLRLYGHSLGDISSNLISFTEVDDAEAVHNSTNCLELTKDLVVQRLVNVSRGNTSGMLVVITKFLRRSENMKLYALCTRPRADGPWTRWTDKDSLLFMVEEHGRFLPLWLHILLVMVLLVLSGIFSGLNLGLMALDPMELRIVQNCGTEKERKYARKIEPIRRKGNYLLCSLLLGNVLVNTSLTILLDNLIGSGIMAVASSTIGIVIFGEILPQALCSRHGLAVGANTIVLTKVFMLLTFPLSFPISKLLDFVLGQEIRTVYNREKLMEMLKVTEPYNDLVKEELNMIQGALELRTKTVEDIMTQLHDCFMIRSDAILDFNTMSEIMESGYTRIPVFEDEQSNIVDILYVKDLAFVDPDDCTPLKTITRFYNHPVHFVFHDTKLDAMLEEFKKGKSHLAIVQKVNNEGEGDPFYEVLGLVTLEDVIEEIIKSEILDESDMYTDNRTRKRVSVKNKRDFSAFKDTDNELKVKISPQLLLAAHRFLATEVPQFSPSLMSEKILLRLLKYPDVIQELRFNEHNRYCVRHYLYTRNKPADCFVLILQGKVEVEAGKENMKFETGAFSYYGTMALSVAPPDRSPALPTPLSRSASLSYPDRNTDLTSTSLAGSNQFGSCILGQYVSDFSVRALTDLQYIKITRQQYQNGLMASRMDNSPQPTFDGCATCSENFMERPELPPVDETTTLLNERNSLLHRASEEETI

SEQ ID NO: 9 - protein, 751 aa, Mus musculus
  mat_peptide (1)..(751): amiloride-sensitive amine oxidase [copper- containing]
MSLAFGWAAVILLLQTADTASAVTTPHDKARIFADLSPQEIKAVHSFLMSRKELGLESSKNLTLAKNSVFLIEMLLPKKKNVLKFLDEGRKSPVREARAIIFFGAQDHPNVTEFAVGPLPRPCYVQALSPRPGHHLSWSSRPISTAEYDLLYHMLNRAITPLHQFFLDTTGFSFLGCDDRFLTFTDVAPRGVESGQRRSWLIVQRYVEGYFLHPTGLEILVDHSSTDVQDWRVEQLWYNGKFYNSPEELAQKYAVGEVEAVVLEEVVLEDPLPGATEQPPLFSSYKPRGEFHTPVTVAGPHVVQPSGPRYKLEGNVVLYGDWSFSYRLRSSSGLQIFNVLFGGERVAYEVSVQEAVALYGGHTPAGMQTKYIDVGWGLGSVTHELAPGIDCPETATFLDAFHYYDSDGPVLYPRALCLFEMPTGVPLRRHFDSNFKGGFNFYAGLKGYVLVLRTTSTVYNYDYIWDFIFYPNGVMETKMHATGYVHATFYTPEGLRHGTRLQTHLLGNIHTHLVHYRVDLDVAGTKNSFRTLKTKLENITNPWSPSHSLVQPTLEQTQYSHEHQAAFRFGQTLPKYLLFSSPQKNRWGHRRSYRLQIHSMAEQVLPPGWQEERAVTWARYPLAVTKYRESERYSSSLYNQNDPWDPPVVFEEFLRNNENIENEDLVAWVTVGFLHIPHSEDVPNTATPGNCVGFLIRPFNFFEEDPSLASRDTVIVWPQDNGLNHVQRWIPENRDCLVSPPFSYNGTYKPV

SEQ ID NO: 10 - protein, 1157 aa, Mus musculus
  mat_peptide (1)..(1157): Hephaestin
MKAGHLLWALLLMHSLWSIPTDGAIRNYYLGIQDMQWNYAPKGRNVITNQTLNNDTVASSFLKSGKNRIGSSYKKTVYKEYSDGTYTEEIAKPAWLGFLGPLLQAEVGDVILIHLKNFASRPYTIHPHGVFYEKDSEGSLYPDGSSGYLKADDSVPPGGSHVYNWSIPESHAPTEADPACLTWIYHSHVDAPRDIATGLIGPLITCKRGTLDGNSPPQRKDVDHNFFLLFSVIDENLSWHLDDNIATYCSDPASVDKEDGAFQDSNRMHAINGFVFGNLPELSMCAQKHVAWHLFGMGNEIDVHTAFFHGQMLSIRGHHTDVANIFPATFVTAEMVPQKSGTWLISCEVNSHLRSGMQAFYKVDSCSMDPPVDQLTGKVRQYFIQAHEIQWDYGPIGYDGRTGKSLREPGSGPDKYFQKSSSRIGGTYWKVRYEAFQDETFQERVHQEEETHLGILGPVIRAEVGDTIQVVFYNRASQPFSIQPHGVFYEKNSEGTVYNDGTSHPKVAKSFEKVTYYWTVPPHAGPTAQDPACLTWMYFSAADPTRDTNSGLVGPLLVCKAGALGADGKQKGVDKEFFLLFTVFDENESWYNNANQAAGMLDSRLLSEDVEGFQDSNRMHAINGFLFSNLPRLDMCKGDTVAWHLLGLGTETDVHGVMFEGNTVQLQGMRKGAVMLFPHTFVTAIMQPDNPGIFEIYCQAGSHREEGMQAIYNVSQCSSHQDSPRQHYQASRVYYIMAEEIEWDYCPDRSWELEWHNTSEKDSYGHVFLSNKDGLLGSKYKKAVFREYTDGTFRIPRPRSGPEEHLGILGPLIRGEVGDILTVVFKNKASRPYSIHAHGVLESNTGGPQAAEPGEVLTYQWNIPERSGPGPSDSACVSWIYYSAVDPIKDMYSGLVGPLVICRNGILEPNGGRNDMDREFALLFLIFDENQSWYLKENIATYGPQESSHVNLKDATFLESNKMHAINGKLYANLRGLTVYQGERVAWYMLAMGQDTDIHTVHFHAESFLYQNGQSYRADVVDLFPGTFEVVEMVASNPGTWLMHCHVTDHVHAGMETIFTVLSHEEHFSTMTTITKEIGKAVILRDIGGDNVKMLGMNIPIKDVEILSSALIAICVLLLLIALALGGVVWYQHRQRKLRRNRRSILDDSFKLLSLKQ

SEQ ID NO: 11 - protein, 697 aa, Mus musculus
  mat_peptide (1)..(697): glutamine-fructose-6-phosphate aminotransferase [isomerizing] 1
MCGIFAYLNYHVPRTRREILETLIKGLQRLEYRGYDSAGVGLDGGNDKDWEANACKIQLIKKKGKVKALDEEVHKQQDMDLDIEFDVHLGIAHTRWATHGEPNPVNSHPQRSDKNNEFIVIHNGIITNYKDLKKFLESKGYDFESETDTETIAKLVKYMYDNWESQDVSFTTLVERVIQQLEGAFALVFKSVHFPGQAVGTRRGSPLLIGVRSEHKLSTDHIPILYRTARTQIGSTWWGSQAERGKDKKGSCGLSRVDSTTCLFPVEEKAVEYYFASDASAVIEHTNRVIFLEDDDVAAVVDGRLSIHRIKRTAGDHPGRAVQTLQMELQQIMKGNFSSFMQKEIFEQPESVVNTMRGRVNFDDYTVNLGGLKDHIKEIQRCRRLILIACGTSYHAGVATRQVLEELTELPVMVELASDFLDRNTPVFRDDVCFFISQSGETADTLMGLRYCKERGALTVGITNTVGSSISRETDCGVHINAGPEIGVASTKAYTSQFVSLVMFALMMCDDRISMQERRKEIMLGLKRLPDLIKEVLSMDDEIQKLATELYHQKSVLIMGRGYHYATCLEGALKIKEITYMHSEGILAGELKHGPLALVDKLMPVIMIIMRDHTYAKCQNALQQVVARQGRPVVICDKEDTETIKNTKRTIKVPHSVDCLQGILSVIPLQLLAFHLAVLRGYDVDFPRNLAKSVTVE

SEQ ID NO: 12 - protein, 771 aa, Mus musculus
  mat_peptide (1)..(771): polymeric immunoglobulin receptor
MRLYLFTLLVTVFSGVSTKSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSDTHVYTKDIGRNVTIECPFKRENAPSKKSLCKKTNQSCELVIDSTEKVNPSYIGRAKLFMKGTDLTVFYVNISHLTHNDAGLYICQAGEGPSADKKNVDLQVLAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERTRGSSHVNPTDANARAKVALEEEVVDSSISEKENKAIPNPGPFANEREIQNVGDQAQENRASGDAGSADGQSRSSSSKVLFSTLVPLGLVLAVGAIAVWVARVRHRKNVDRMSISSYRTDISMADFKNSRDLGGNDNMGASPDTQQTVIEGKDEIVTTTECTAEPEESKKAKRSSKEEADMAYSAFLLQSSTIAAQVHDGPQEA

SEQ ID NO: 13 - protein, 739 aa, Mus musculus
  mat_peptide (1)..(739): sulfate transporter
MSSENKEQHDLSPRDLPEEAFGFPSELPLETQRRSGTDLRQSETGHGRRAFRRIHMELREKPDTDIKQFVIRELQKSCQCSAAKVRDGAFDFFPVLRWLPKYDLKKNILGDVMSGLIVGILLVPQSIAYSLLAGQEPIYGLYTSFFASIIYFLFGTSRHISVGIFGILCLMIGEVVDRELHKACPDTDATSSSIAVFSSGCVVVNHTLDGLCDKSCYAIKIGSTVTFMAGVYQVAMGFFQVGFVSVYLSDALLSGFVTGASFTILTSQAKYLLGLSLPRSHGVGSVITTWIHIFRNIRNTNICDLITSLLCLLVLVPSKELNEHFKDKLKAPIPVELIVVVAATLASHFGKLNGNYNSSIAGHIPTGFMPPKAPDWSLIPNVAVDAIAISIIGFAITVSLSEMFAKKHGYTVKANQEMYAIGFCNIIPSFFHCITTSAALAKTLVKESTGCQTQLSAIVTALVLLLVLLVIAPLFYSLQKCVLGVITIVNLRGALLKFRDLPKMWRLSRMDTVIWFVTMLSSALLSTEIGLLVGVCFSMFCVILRTQKPKNSLLGLEEESETFESISTYKNLRSKSGIKVFRFIAPLYYINKECFKSALYKKALNPVLVKAAWKKAAKRKLKEEMVTFRGDPDEVSMQLSHDPLEVHTIVIDCSAIQFLDTAGIHTLKEVRRDYEAVGIQVLLAQCNPSVRDSLARGEYCKKEEETLLFYSLSEAVAFAEDSQNQKGVCVVNGLSLSGD

SEQ ID NO: 14 - protein, 132 aa, Mus musculus
  mat_peptide (1)..(132): metal transporter CNNM4
MTGELEVKNMDMKPGSTLKITGSIADGTDGFVINLGQGTDKLNLHFNPRFSESTIVCNSLDGSNWGQEQREDHLCFSPGSEVKFTVTFESDKFKVKLPDGHELTFPNRLGHSHLSYLSVRGGFNMSSFKLKE

SEQ ID NO: 15 - protein, 5405 aa, Homo sapiens
  mat_peptide (1)..(5405): IgGFc-binding protein
MGALWSWWILWAGATLLWGLTQEASVDLKNTGREEFLTAFLQNYQLAYSKAYPRLLISSLSESPASVSILSQADNTSKKVTVRPGESVMVNISAKAEMIGSKIFQHAVVIHSDYAISVQALNAKPDTAELTLLRPIQALGTEYFVLTPPGTSARNVKEFAVVAGAAGASVSVTLKGSVTFNGKFYPAGDVLRVTLQPYNVAQLQSSVDLSGSKVTASSPVAVLSGHSCAQKHTTCNHVVEQLLPTSAWGTHYVVPTLASQSRYDLAFVVASQATKLTYNHGGITGSRGLQAGDVVEFEVRPSWPLYLSANVGIQVLLFGTGAIRNEVTYDPYLVLIPDVAAYCPAYVVKSVPGCEGVALVVAQTKAISGLTIDGHAVGAKLTWEAVPGSEFSYAEVELGTADMIHTAEATTNLGLLTFGLAKAIGYATAADCGRTVLSPVEPSCEGMQCAAGQRCQVVGGKAGCVAESTAVCRAQGDPHYTTFDGRRYDMMGTCSYTMVELCSEDDTLPAFSVEAKNEHRGSRRVSYVGLVTVRAYSHSVSLTRGEVGFVLVDNQRSRLPVSLSEGRLRVYQSGPRAVVELVFGLVVTYDWDCQLALSLPARFQDQVCGLCGNYNGDPADDFLTPDGALAPDAVEFASSWKLDDGDYLCEDGCQNNCPACTPGQAQHYEGDRLCGMLTKLDGPFAVCHDTLDPRPFLEQCVYDLCVVGGERLSLCRGLSAYAQACLELGISVGDWRSPANCPLSCPANSRYELCGPACPTSCNGAAAPSNCSGRPCVEGCVCLPGFVASGGACVPASSCGCTFQGLQLAPGQEVWADELCQRRCTCNGATHQVTCRDKQSCPAGERCSVQNGLLGCYPDRFGTCQGSGDPHYVSFDGRRFDFMGTCTYLLVGSCGQNAALPAFRVLVENEHRGSQTVSYTRAVRVEARGVKVAVRREYPGQVLVDDVLQYLPFQAADGQVQVFRQGRDAVVRTDFGLTVTYDWNARVTAKVPSSYAEALCGLCGNFNGDPADDLALRGGGQAANALAFGNSWQEETRPGCGATEPGDCPKLDSLVAQQLQSKNECGILADPKGPFRECHSKLDPQGAVRDCVYDRCLLPGQSGPLCDALATYAAACQAAGATVHPWRSEELCPLSCPPHSHYEACSYGCPLSCGDLPVPGGCGSECHEGCVCDEGFALSGESCLPLASCGCVHQGTYHPPGQTFYPGPGCDSLCHCQEGGLVSCESSSCGPHEACQPSGGSLGCVAVGSSTCQASGDPHYTTFDGRRFDFMGTCVYVLAQTCGTRPGLHRFAVLQENVAWGNGRVSVTRVITVQVANFTLRLEQRQWKVTVNGVDMKLPVVLANGQIRASQHGSDVVIETDFGLRVAYDLVYYVRVTVPGNYYQQMCGLCGNYNGDPKDDFQKPNGSQAGNANEFGNSWEEVVPDSPCLPPTPCPPGSEDCIPSHKCPPELEKKYQKEEFCGLLSSPTGPLSSCHKLVDPQGPLKDCIFDLCLGGGNLSILCSNIHAYVSACQAAGGHVEPWRTETFCPMECPPNSHYELCADTCSLGCSALSAPPQCQDGCAEGCQCDSGFLYNGQACVPIQQCGCYHNGVYYEPEQTVLIDNCRQQCTCHAGKGMVCQEHSCKPGQVCQPSGGILSCVTKDPCHGVTCRPQETCKEQGGQGVCLPNYEATCWLWGDPHYHSFDGRKFDFQGTCNYVLATTGCPGVSTQGLTPFTVTTKNQNRGNPAVSYVRVVTVAALGTNISIHKDEIGKVRVNGVLTALPVSVADGRISVTQGASKALLVADFGLQVSYDWNWRVDVTLPSSYHGAVCGLCGNMDRNPNNDQVFPNGTLAPSIPIWGGSWRAPGWDPLCWDECRGSCPTCPEDRLEQYEGPGFCGPLAPGTGGPFTTCHAHVPPESFFKGCVLDVCMGGGDRDILCKALASYVAACQAAGVVIEDWRAQVGCEITCPENSHYEVCGSPCPASCPSPAPLTTPAVCEGPCVEGCQCDAGFVLSADRCVPLNNGCGCWANGTYHEAGSEFWADGTCSQWCRCGPGGGSLVCTPASCGLGEVCGLLPSGQHGCQPVSTAECQAWGDPHYVTLDGHRFNFQGTCEYLLSAPCHGPPLGAENFTVTVANEHRGSQAVSYTRSVTLQIYNHSLTLSARWPRKLQVDGVFVTLPFQLDSLLHAHLSGADVVVTTTSGLSLAFDGDSFVRLRVPAAYAGSLCGLCGNYNQDPADDLKAVGGKPAGWQVGGAQGCGECVSKPCPSPCTPEQQESFGGPDACGVISATDGPLAPCHGLVPPAQYFQGCLLDACQVQGHPGGLCPAVATYVAACQAAGAQLREWRRPDFCPFQCPAHSHYELCGDSCPGSCPSLSAPEGCESACREGCVCDAGFVLSGDTCVPVGQCGCLHDDRYYPLGQTFYPGPGCDSLCRCREGGEVSCEPSSCGPHETCRPSGGSLGCVAVGSTTCQASGDPHYTTFDGRRFDFMGTCVYVLAQTCGTRPGLHRFAVLQENVAWGNGRVSVTRVITVQVANFTLRLEQRQWKVTVNGVDMKLPVVLANGQIRASQHGSDVVIETDFGLRVAYDLVYYVRVTVPGNYYQLMCGLCGNYNGDPKDDFQKPNGSQAGNANEFGNSWEEVVPDSPCLPPPTCPPGSEGCIPSEECPPELEKKYQKEEFCGLLSSPTGPLSSCHKLVDPQGPLKDCIFDLCLGGGNLSILCSNIHAYVSACQAAGGQVEPWRNETFCPMECPQNSHYELCADTCSLGCSALSAPLQCPDGCAEGCQCDSGFLYNGQACVPIQQCGCYHNGAYYEPEQTVLIDNCRQQCTCHVGKVVVCQEHSCKPGQVCQPSGGILSCVNKDPCHGVTCRPQETCKEQGGQGVCLPNYEATCWLWGDPHYHSFDGRKFDFQGTCNYVLATTGCPGVSTQGLTPFTVTTKNQNRGNPAVSYVRVVTVAALGTNISIHKDEIGKVRVNGVLTALPVSVADGRISVTQGASKALLVADFGLQVSYDWNWRVDVTLPSSYHGAVCGLCGNMDRNPNNDQVFPNGTLAPSIPIWGGSWRAPGWDPLCWDECRGSCPTCPEDRLEQYEGPGFCGPLAPGTGGPFTTCHAHVPPESFFKGCVLDVCMGGGDRDILCKALASYVAACQAAGVVIEDWRAQVGCEITCPENSHYEVCGPPCPASCPSPAPLTTPAVCEGPCVEGCQCDAGFVLSADRCVPLNNGCGCWANGTYHEAGSEFWADGTCSQWCRCGPGGGSLVCTPASCGLGEVCGLLPSGQHGCQPVSTAECQAWGDPHYVTLDGHRFDFQGTCEYLLSAPCHGPPLGAENFTVTVANEHRGSQAVSYTRSVTLQIYNHSLTLSARWPRKLQVDGVFVTLPFQLDSLLHAHLSGADVVVTTTSGLSLAFDGDSFVRLRVPAAYAGSLCGLCGNYNQDPADDLKAVGGKPAGWQVGGAQGCGECVSKPCPSPCTPEQQESFGGPDACGVISATDGPLAPCHGLVPPAQYFQGCLLDACQVQGHPGGLCPAVATYVAACQAAGAQLREWRRPDFCPFQCPAHSHYELCGDSCPGSCPSLSAPEGCESACREGCVCDAGFVLSGDTCVPVGQCGCLHDDRYYPLGQTFYPGPGCDSLCRCREGGEVSCEPSSCGPHETCRPSGGSLGCVAVGSTTCQASGDPHYTTFDGHRFDFMGTCVYVLAQTCGTRPGLHRFAVLQENVAWGNGRVSVTRVITVQVANFTLRLEQRQWKVTVNGVDMKLPVVLANGQIRASQHGSDVVIETDFGLRVAYDLVYYVRVTVPGNYYQLMCGLCGNYNGDPKDDFQKPNGSQAGNANEFGNSWEEVVPDSPCLPPPTCPPGSAGCIPSDKCPPELEKKYQKEEFCGLLSSPTGPLSSCHKLVDPQGPLKDCIFDLCLGGGNLSILCSNIHAYVSACQAAGGHVEPWRNETFCPMECPQNSHYELCADTCSLGCSALSAPLQCPDGCAEGCQCDSGFLYNGQACVPIQQCGCYHNGVYYEPEQTVLIDNCRQQCTCHVGKVVVCQEHSCKPGQVCQPSGGILSCVTKDPCHGVTCRPQETCKEQGGQGVCLPNYEATCWLWGDPHYHSFDGRKFDFQGTCNYVLATTGCPGVSTQGLTPFTVTTKNQNRGNPAVSYVRVVTVAALGTNISIHKDEIGKVRVNGVLTALPVSVADGRISVAQGASKALLVADFGLQVSYDWNWRVDVTLPSSYHGAVCGLCGNMDRNPNNDQVFPNGTLAPSIPIWGGSWRAPGWDPLCWDECRGSCPTCPEDRLEQYEGPGFCGPLSSGTGGPFTTCHAHVPPESFFKGCVLDVCMGGGDRDILCKALASYVAACQAAGVVIEDWRAQVGCEITCPENSHYEVCGPPCPASCPSPAPLTTPAVCEGPCVEGCQCDAGFVLSADRCVPLNNGCGCWANGTYHEAGSEFWADGTCSQWCRCGPGGGSLVCTPASCGLGEVCGLLPSGQHGCQPVSTAECQAWGDPHYVTLDGHRFDFQGTCEYLLSAPCHGPPLGAENFTVTVANEHRGSQAVSYTRSVTLQIYNHSLTLSARWPRKLQVDGVFVALPFQLDSLLHAHLSGADVVVTTTSGLSLAFDGDSFVRLRVPAAYAASLCGLCGNYNQDPADDLKAVGGKPAGWQVGGAQGCGECVSKPCPSPCTPEQQESFGGPDACGVISATDGPLAPCHGLVPPAQYFQGCLLDACQVQGHPGGLCPAVATYVAACQAAGAQLGEWRRPDFCPLQCPAHSHYELCGDSCPVSCPSLSAPEGCESACREGCVCDAGFVLSGDTCVPVGQCGCLHDGRYYPLGEVFYPGPECERRCECGPGGHVTCQEGAACGPHEECRLEDGVQACHATGCGRCLANGGIHYITLDGRVYDLHGSCSYVLAQVCHPKPGDEDFSIVLEKNAAGDLQRLLVTVAGQVVSLAQGQQVTVDGEAVALPVAVGRVRVTAEGRNMVLQTTKGLRLLFDGDAHLLMSIPSPFRGRLCGLCGNFNGNWSDDFVLPNGSAASSVETFGAAWRAPGSSKGCGEGCGPQGCPVCLAEETAPYESNEACGQLRNPQGPFATCQAVLSPSEYFRQCVYDLCAQKGDKAFLCRSLAAYTAACQAAGVAVKPWRTDSFCPLHCPAHSHYSICTRTCQGSCAALSGLTGCTTRCFEGCECDDRFLLSQGVCIPVQDCGCTHNGRYLPVNSSLLTSDCSERCSCSSSSGLTCQAAGCPPGRVCEVKAEARNCWATRGLCVLSVGANLTTFDGARGATTSPGVYELSSRCPGLQNTIPWYRVVAEVQICHGKTEAVGQVHIFFQDGMVTLTPNKGVWVNGLRVDLPAEKLASVSVSRTPDGSLLVRQKAGVQVWLGANGKVAVIVSNDHAGKLCGACGNFDGDQTNDWHDSQEKPAMEKWRAQDFSPCYG

SEQ ID NO: 16 - protein, 323 aa, Homo sapiens
  mat_peptide (1)..(323): galectin-4
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIGGQPLRPQGPPMMPPYPGPGHCHQQLNSLPTMEGPPTFNPPVPYFGRLQGGLTARRTIIIKGYVPPTGKSFAINFKVGSSGDIALHINPRMGNGTVVRNSLLNGSWGSEEKKITHNPFGPGQFFDLSIRCGLDRFKVYANGQHLFDFAHRLSAFQRVDTLEIQGDVTLSYVQI

SEQ ID NO: 17 - protein, 5179 aa, Homo sapiens
  mat_peptide (1)..(5179): mucin-2
MGLPLARLAAVCLALSLAGGSELQTEGRTRYHGRNVCSTWGNFHYKTFDGDVFRFPGLCDYNFASDCRGSYKEFAVHLKRGPGQAEAPAGVESILLTIKDDTIYLTRHLAVLNGAVVSTPHYSPGLLIEKSDAYTKVYSRAGLTLMWNREDALMLELDTKFRNHTCGLCGDYNGLQSYSEFLSDGVLFSPLEFGNMQKINQPDVVCEDPEEEVAPASCSEHRAECERLLTAEAFADCQDLVPLEPYLRACQQDRCRCPGGDTCVCSTVAEFSRQCSHAGGRPGNWRTATLCPKTCPGNLVYLESGSPCMDTCSHLEVSSLCEEHRMDGCFCPEGTVYDDIGDSGCVPVSQCHCRLHGHLYTPGQEITNDCEQCVCNAGRWVCKDLPCPGTCALEGGSHITTFDGKTYTFHGDCYYVLAKGDHNDSYALLGELAPCGSTDKQTCLKTVVLLADKKKNAVVFKSDGSVLLNQLQVNLPHVTASFSVFRPSSYHIMVSMAIGVRLQVQLAPVMQLFVTLDQASQGQVQGLCGNFNGLEGDDFKTASGLVEATGAGFANTWKAQSTCHDKLDWLDDPCSLNIESANYAEHWCSLLKKTETPFGRCHSAVDPAEYYKRCKYDTCNCQNNEDCLCAALSSYARACTAKGVMLWGWREHVCNKDVGSCPNSQVFLYNLTTCQQTCRSLSEADSHCLEGFAPVDGCGCPDHTFLDEKGRCVPLAKCSCYHRGLYLEAGDVVVRQEERCVCRDGRLHCRQIRLIGQSCTAPKIHMDCSNLTALATSKPRALSCQTLAAGYYHTECVSGCVCPDGLMDDGRGGCVVEKECPCVHNNDLYSSGAKIKVDCNTCTCKRGRWVCTQAVCHGTCSIYGSGHYITFDGKYYDFDGHCSYVAVQDYCGQNSSLGSFSIITENVPCGTTGVTCSKAIKIFMGRTELKLEDKHRVVIQRDEGHHVAYTTREVGQYLVVESSTGIIVIWDKRTTVFIKLAPSYKGTVCGLCGNFDHRSNNDFTTRDHMVVSSELDFGNSWKEAPTCPDVSTNPEPCSLNPHRRSWAEKQCSILKSSVFSICHSKVDPKPFYEACVHDSCSCDTGGDCECFCSAVASYAQECTKEGACVFWRTPDLCPIFCDYYNPPHECEWHYEPCGNRSFETCRTINGIHSNISVSYLEGCYPRCPKDRPIYEEDLKKCVTADKCGCYVEDTHYPPGASVPTEETCKSCVCTNSSQVVCRPEEGKILNQTQDGAFCYWEICGPNGTVEKHFNICSITTRPSTLTTFTTITLPTTPTSFTTTTTTTTPTSSTVLSTTPKLCCLWSDWINEDHPSSGSDDGDREPFDGVCGAPEDIECRSVKDPHLSLEQHGQKVQCDVSVGFICKNEDQFGNGPFGLCYDYKIRVNCCWPMDKCITTPSPPTTTPSPPPTTTTTLPPTTTPSPPTTTTTTPPPTTTPSPPITTTTTPLPTTTPSPPISTTTTPPPTTTPSPPTTTPSPPTTTPSPPTTTTTTPPPTTTPSPPMTTPITPPASTTTLPPTTTPSPPTTTTTTPPPTTTPSPPTTTPITPPTSTTTLPPTTTPSPPPTTTTTPPPTTTPSPPTTTTPSPPTITTTTPPPTTTPSPPTTTTTTPPPTTTPSPPTTTPITPPTSTTTLPPTTTPSPPPTTTTTPPPTTTPSPPTTTTPSPPITTTTTPPPTTTPSSPITTTPSPPTTTMTTPSPTTTPSSPITTTTTPSSTTTPSPPPTTMTTPSPTTTPSPPTTTMTTLPPTTTSSPLTTTPLPPSITPPTFSPFSTTTPTTPCVPLCNWTGWLDSGKPNFHKPGGDTELIGDVCGPGWAANISCRATMYPDVPIGQLGQTVVCDVSVGLICKNEDQKPGGVIPMAFCLNYEINVQCCECVTQPTTMTTTTTENPTPPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTGPPTHTSTAPIAELTTSNPPPESSTPQTSRSTSSPLTESTTLLSTLPPAIEMTSTAPPSTPTAPTTTSGGHTLSPPPSTTTSPPGTPTRGTTTGSSSAPTPSTVQTTTTSAWTPTPTPLSTPSIIRTTGLRPYPSSVLICCVLNDTYYAPGEEVYNGTYGDTCYFVNCSLSCTLEFYNWSCPSTPSPTPTPSKSTPTPSKPSSTPSKPTPGTKPPECPDFDPPRQENETWWLCDCFMATCKYNNTVEIVKVECEPPPMPTCSNGLQPVRVEDPDGCCWHWECDCYCTGWGDPHYVTFDGLYYSYQGNCTYVLVEEISPSVDNFGVYIDNYHCDPNDKVSCPRTLIVRHETQEVLIKTVHMMPMQVQVQVNRQAVALPYKKYGLEVYQSGINYVVDIPELGVLVSYNGLSFSVRLPYHRFGNNTKGQCGTCTNTTSDDCILPSGEIVSNCEAAADQWLVNDPSKPHCPHSSSTTKRPAVTVPGGGKTTPHKDCTPSPLCQLIKDSLFAQCHALVPPQHYYDACVFDSCFMPGSSLECASLQAYAALCAQQNICLDWRNHTHGACLVECPSHREYQACGPAEEPTCKSSSSQQNNTVLVEGCFCPEGTMNYAPGFDVCVKTCGCVGPDNVPREFGEHFEFDCKNCVCLEGGSGIICQPKRCSQKPVTHCVEDGTYLATEVNPADTCCNITVCKCNTSLCKEKPSVCPLGFEVKSKMVPGRCCPFYWCESKGVCVHGNAEYQPGSPVYSSKCQDCVCTDKVDNNTLLNVIACTHVPCNTSCSPGFELMEAPGECCKKCEQTHCIIKRPDNQHVILKPGDFKSDPKNNCTFFSCVKIHNQLISSVSNITCPNFDASICIPGSITFMPNGCCKTCTPRNETRVPCSTVPVTTEVSYAGCTKTVLMNHCSGSCGTFVMYSAKAQALDHSCSCCKEEKTSQREVVLSCPNGGSLTHTYTHIESCQCQDTVCGLPTGTSRRARRSPRHLGSG

SEQ ID NO: 18 - protein, 175 aa, Homo sapiens
  mat_peptide (1)..(175): anterior gradient protein 2 homolog
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 19 - protein, 832 aa, Homo sapiens
  mat_peptide (1)..(832): cadherin-17
MILQAHLHSLCLLMLYLATGYGQEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMAVGILLTTLLVIGIILAVVFIRIKKDKGKDNVESAQASEVKPLRS

SEQ ID NO: 20 - protein, 167 aa, Homo sapiens
  mat_peptide (1)..(167): zymogen granule membrane protein 16
MLTVALLALLCASASGNAIQARSSSYSGEYGGGGGKRFSHSGNQLDGPITALRVRVNTYYIVGLQVRYGKVWSDYVGGRNGDLEEIFLHPGESVIQVSGKYKWYLKKLLFVTDKGRYLSFGKDSGTSFNAVPLHPNTVLRFISGRSGSLIDAIGLHWDVYPSSCSRC

SEQ ID NO: 21 - protein, 775 aa, Homo sapiens
  mat_peptide (1)..(775): metal transporter CNNM4
MAPVGGGGRPVGGPARGRLLLAAPVLLVLLWALGARGQGSPQQGTIVGMRLASCNKSCGTNPDGIIFVSEGSTVNLRLYGYSLGNISSNLISFTEVDDAETLHKSTSCLELTKDLVVQQLVNVSRGNTSGVLVVLTKFLRRSESMKLYALCTRAQPDGPWLKWTDKDSLLFMVEEPGRFLPLWLHILLITVLLVLSGIFSGLNLGLMALDPMELRIVQNCGTEKERRYARKIEPIRRKGNYLLCSLLLGNVLVNTSLTILLDNLIGSGLMAVASSTIGIVIFGEILPQALCSRHGLAVGANTILLTKFFMLLTFPLSFPISKLLDFFLGQEIRTVYNREKLMEMLKVTEPYNDLVKEELNMIQGALELRTKTVEDIMTQLQDCFMIRSDAILDFNTMSEIMESGYTRIPVFEDEQSNIVDILYVKDLAFVDPDDCTPLKTITRFYNHPVHFVFHDTKLDAMLEEFKKGKSHLAIVQKVNNEGEGDPFYEVLGLVTLEDVIEEIIKSEILDESDMYTDNRSRKRVSEKNKRDFSAFKDADNELKVKISPQLLLAAHRFLATEVSQFSPSLISEKILLRLLKYPDVIQELKFDEHNKYYARHYLYTRNKPADYFILILQGKVEVEAGKENMKFETGAFSYYGTMALTSVPSDRSPAHPTPLSRSASLSYPDRTDVSTAATLAGSSNQFGSSVLGQYISDFSVRALVDLQYIKITRQQYQNGLLASRMENSPQFPIDGCTTHMENLAEKSELPVVDETTTLLNERNSLLHKASHENAI

SEQ ID NO: 22 - protein, 751 aa, Homo sapiens
  mat_peptide (1)..(751): amiloride-sensitive amine oxidase [copper- containing]
MPALGWAVAAILMLQTAMAEPSPGTLPRKAGVFSDLSNQELKAVHSFLWSKKELRLQPSSTTTMAKNTVFLIEMLLPKKYHVLRFLDKGERHPVREARAVIFFGDQEHPNVTEFAVGPLPGPCYMRALSPRPGYQSSWASRPISTAEYALLYHTLQEATKPLHQFFLNTTGFSFQDCHDRCLAFTDVAPRGVASGQRRSWLIIQRYVEGYFLHPTGLELLVDHGSTDAGHWAVEQVWYNGKFYGSPEELARKYADGEVDVVVLEDPLPGGKGHDSTEEPPLFSSHKPRGDFPSPIHVSGPRLVQPHGPRFRLEGNAVLYGGWSFAFRLRSSSGLQVLNVHFGGERIAYEVSVQEAVALYGGHTPAGMQTKYLDVGWGLGSVTHELAPGIDCPETATFLDTFHYYDADDPVHYPRALCLFEMPTGVPLRRHFNSNFKGGFNFYAGLKGQVLVLRTTSTVYNYDYIWDFIFYPNGVMEAKMHATGYVHATFYTPEGLRHGTRLHTHLIGNIHTHLVHYRVDLDVAGTKNSFQTLQMKLENITNPWSPRHRVVQPTLEQTQYSWERQAAFRFKRKLPKYLLFTSPQENPWGHKRTYRLQIHSMADQVLPPGWQEEQAITWARYPLAVTKYRESELCSSSIYHQNDPWHPPVVFEQFLHNNENIENEDLVAWVTVGFLHIPHSEDIPNTATPGNSVGFLLRPFNFFPEDPSLASRDTVIVWPRDNGPNYVQRWIPEDRDCSMPPPFSYNGTYRPV

SEQ ID NO: 23 - protein, 1158 aa, Homo sapiens
  mat_peptide (1)..(1158): Hephaestin
MESGHLLWALLFMQSLWPQLTDGATRVYYLGIRDVQWNYAPKGRNVITNQPLDSDIVASSFLKSDKNRIGGTYKKTIYKEYKDDSYTDEVAQPAWLGFLGPVLQAEVGDVILIHLKNFATRPYTIHPHGVFYEKDSEGSLYPDGSSGPLKADDSVPPGGSHIYNWTIPEGHAPTDADPACLTWIYHSHVDAPRDIATGLIGPLITCKRGALDGNSPPQRQDVDHDFFLLFSVVDENLSWHLNENIATYCSDPASVDKEDETFQESNRMHAINGFVFGNLPELNMCAQKRVAWHLFGMGNEIDVHTAFFHGQMLTTRGHHTDVANIFPATFVTAEMVPWEPGTWLISCQVNSHFRDGMQALYKVKSCSMAPPVDLLTGKVRQYFIEAHEIQWDYGPMGHDGSTGKNLREPGSISDKFFQKSSSRIGGTYWKVRYEAFQDETFQEKMHLEEDRHLGILGPVIRAEVGDTIQVVFYNRASQPFSMQPHGVFYEKDYEGTVYNDGSSYPGLVAKPFEKVTYRWTVPPHAGPTAQDPACLTWMYFSAADPIRDTNSGLVGPLLVCRAGALGADGKQKGVDKEFFLLFTVLDENKSWYSNANQAAAMLDFRLLSEDIEGFQDSNRMHAINGFLFSNLPRLDMCKGDTVAWHLLGLGTETDVHGVMFQGNTVQLQGMRKGAAMLFPHTFVMAIMQPDNLGTFEIYCQAGSHREAGMRAIYNVSQCPGHQATPRQRYQAARIYYIMAEEVEWDYCPDRSWEREWHNQSEKDSYGYIFLSNKDGLLGSRYKKAVFREYTDGTFRIPRPRTGPEEHLGILGPLIKGEVGDILTVVFKNNASRPYSVHAHGVLESTTVWPLAAEPGEVVTYQWNIPERSGPGPNDSACVSWIYYSAVDPIKDMYSGLVGPLAICQKGILEPHGGRSDMDREFALLFLIFDENKSWYLEENVATHGSQDPGSINLQDETFLESNKMHAINGKLYANLRGLTMYQGERVAWYMLAMGQDVDLHTIHFHAESFLYRNGENYRADVVDLFPGTFEVVEMVASNPGTWLMHCHVTDHVHAGMETLFTVFSRTEHLSPLTVITKETEKAVPPRDIEEGNVKMLGMQIPIKNVEMLASVLVAISVTLLLVVLALGGVVWYQHRQRKLRRNRRSILDDSFKLLSFKQ

SEQ ID NO: 24 - protein, 699 aa, Homo sapiens
  mat_peptide (1)..(699): glutamine-fructose-6-phosphate aminotransferase [isomerizing] 1
MCGIFAYLNYHVPRTRREILETLIKGLQRLEYRGYDSAGVGFDGGNDKDWEANACKIQLIKKKGKVKALDEEVHKQQDMDLDIEFDVHLGIAHTRWATHGEPSPVNSHPQRSDKNNEFIVIHNGIITNYKDLKKFLESKGYDFESETDTETIAKLVKYMYDNRESQDTSFTTLVERVIQQLEGAFALVFKSVHFPGQAVGTRRGSPLLIGVRSEHKLSTDHIPILYRTARTQIGSKFTRWGSQGERGKDKKGSCNLSRVDSTTCLFPVEEKAVEYYFASDASAVIEHTNRVIFLEDDDVAAVVDGRLSIHRIKRTAGDHPGRAVQTLQMELQQIMKGNFSSFMQKEIFEQPESVVNTMRGRVNFDDYTVNLGGLKDHIKEIQRCRRLILIACGTSYHAGVATRQVLEELTELPVMVELASDFLDRNTPVFRDDVCFFLSQSGETADTLMGLRYCKERGALTVGITNTVGSSISRETDCGVHINAGPEIGVASTKAYTSQFVSLVMFALMMCDDRISMQERRKEIMLGLKRLPDLIKEVLSMDDEIQKLATELYHQKSVLIMGRGYHYATCLEGALKIKEITYMHSEGILAGELKHGPLALVDKLMPVIMIIMRDHTYAKCQNALQQVVARQGRPVVICDKEDTETIKNTKRTIKVPHSVDCLQGILSVIPLQLLAFHLAVLRGYDVDFPRNLAKSVTVE

SEQ ID NO: 25 - protein, 764 aa, Homo sapiens
  mat_peptide (1)..(764): polymeric immunoglobulin receptor
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 26 - protein, 739 aa, Homo sapiens
  mat_peptide (1)..(739): sulfate transporter
MSSESKEQHNVSPRDSAEGNDSYPSGIHLELQRESSTDFKQFETNDQCRPYHRILIERQEKSDTNFKEFVIKKLQKNCQCSPAKAKNMILGFLPVLQWLPKYDLKKNILGDVMSGLIVGILLVPQSIAYSLLAGQEPVYGLYTSFFASIIYFLLGTSRHISVGIFGVLCLMIGETVDRELQKAGYDNAHSAPSLGMVSNGSTLLNHTSDRICDKSCYAIMVGSTVTFIAGVYQVAMGFFQVGFVSVYLSDALLSGFVTGASFTILTSQAKYLLGLNLPRTNGVGSLITTWIHVFRNIHKTNLCDLITSLLCLLVLLPTKELNEHFKSKLKAPIPIELVVVVAATLASHFGKLHENYNSSIAGHIPTGFMPPKVPEWNLIPSVAVDAIAISIIGFAITVSLSEMFAKKHGYTVKANQEMYAIGFCNIIPSFFHCFTTSAALAKTLVKESTGCHTQLSGVVTALVLLLVLLVIAPLFYSLQKSVLGVITIVNLRGALRKFRDLPKMWSISRMDTVIWFVTMLSSALLSTEIGLLVGVCFSIFCVILRTQKPKSSLLGLVEESEVFESVSAYKNLQIKPGIKIFRFVAPLYYINKECFKSALYKQTVNPILIKVAWKKAAKRKIKEKVVTLGGIQDEMSVQLSHDPLELHTIVIDCSAIQFLDTAGIHTLKEVRRDYEAIGIQVLLAQCNPTVRDSLTNGEYCKKEEENLLFYSVYEAMAFAEVSKNQKGVCVPNGLSLSSD

SEQ ID NO: 27 - protein, 216 aa, Homo sapiens
  mat_peptide (1)..(216): cadherin-17 domain 1-2
PLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPV

SEQ ID NO: 28 - protein, 7 aa, Artificial Sequence
  Anti-human cadherin-17 antibody AV17-HCDR3
RPVMFDY

SEQ ID NO: 29 - protein, 6 aa, Artificial Sequence
  AV17-HCDR1
SSYAMS

SEQ ID NO: 30 - protein, 17 aa, Artificial Sequence
  AV17-HCDR2
AISGSGGSTYYADSVKG

SEQ ID NO: 31 - protein, 11 aa, Artificial Sequence
  AV17-LCDR1
QGDSLRSYYAS

SEQ ID NO: 32 - protein, 7 aa, Artificial Sequence
  AV17-LCDR2
GKNNRPS

SEQ ID NO: 33 - protein, 10 aa, Artificial Sequence
  AV17-LCDR3
NSSLPSPQVV

SEQ ID NO: 34 - protein, 116 aa, Artificial Sequence
  AV17-VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRPVMFDYWGQGTLVTVSS

SEQ ID NO: 35 - protein, 109 aa, Artificial Sequence
  AV17-VL
SSELTQDPAVSVALGQTVRITCQGDSLRSYYASWYQQKPGQAPVLVIYGKNNRPSGIPDRFSGSSSGNTASLTITGAQAEDEADYYCNSSLPSPQVVVFGGGTKLTVLG

SEQ ID NO: 36 - protein, 239 aa, Artificial Sequence
  AV17-full-length scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRPVMFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSLPSPQVVVFGGGTKLTVLG

SEQ ID NO: 37 - DNA, 717 bp, Artificial Sequence
  AV17 full-length scFv
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaacgtccggtgatgtttgactactggggccagggaaccctggtcaccgtctcgagtggtggaggcggttcaggcggaggtggctctggcggtggcggatcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctttgccctcgcctcaggttgtggtattcggcggagggaccaagctgaccgtcctaggc

SEQ ID NO: 38 - DNA, 348 bp, Artificial Sequence
  AV17-VH
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaacgtccggtgatgtttgactactggggccagggaaccctggtcaccgtctcgagt

SEQ ID NO: 39 - DNA, 327 bp, Artificial Sequence
  AV17-VL
tcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctttgccctcgcctcaggttgtggtattcggcggagggaccaagctgaccgtcctaggc

SEQ ID NO: 40 - protein, 14 aa, Artificial Sequence
  Amino Acid linker sequence
GGGGSGGGGSGGGG

SEQ ID NO: 41 - DNA, 42 bp, Artificial Sequence
  Nucleic acid linker sequence
ggtggaggcggttcaggcggaggtggctctggcggtggcgga

SEQ ID NO: 42 - protein, 7 aa, Artificial Sequence
  Anti-human cadherin-17 antibody AV17.2 HCDR3
RPHMFDY

SEQ ID NO: 43 - protein, 6 aa, Artificial Sequence
  AV17.2 HCDR1
SSYAMS

SEQ ID NO: 44 - protein, 17 aa, Artificial Sequence
  AV17.2 HCDR2
AISGSGGSTYYADSVKG

SEQ ID NO: 45 - protein, 11 aa, Artificial Sequence
  AV17.2 LCDR1
QGDSLRSYYAS

SEQ ID NO: 46 - protein, 7 aa, Artificial Sequence
  AV17.2 LCDR2
GKNNRPS

SEQ ID NO: 47 - protein, 10 aa, Artificial Sequence
  AV17.2 LCDR3
NSSRPDSNGV

SEQ ID NO: 48 - protein, 116 aa, Artificial Sequence
  AV17.2 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRPHMFDYWGQGTLVTVSS

SEQ ID NO: 49 - protein, 109 aa, Artificial Sequence
  AV17.2 VL
SSELTQDPAVSVALGQTVRITCQGDSLRSYYASWYQQKPGQAPVLVIYGKNNRPSGIPDRFSGSSSGNTASLTITGAQAEDEADYYCNSSRPDSNGVVFGGGTKLTVLG

SEQ ID NO: 50 - protein, 239 aa, Artificial Sequence
  AV17.2 full-length scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRPHMFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSRPDSNGVVFGGGTKLTVLG

SEQ ID NO: 51 - DNA, 717 bp, Artificial Sequence
  AV17.2 full-length scFv
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaacggccgcatatgtttgactactggggccagggaaccctggtcaccgtctcgagtggtggaggcggttcaggcggaggtggctctggcggtggcggatcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctcggcccgattctaatggtgtggtattcggcggagggaccaagctgaccgtcctaggc

SEQ ID NO: 52 - DNA, 348 bp, Artificial Sequence
  AV17.2 VH
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaacggccgcatatgtttgactactggggccagggaaccctggtcaccgtctcgagt

SEQ ID NO: 53 - DNA, 327 bp, Artificial Sequence
  AV17.2 VL
tcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctcggcccgattctaatggtgtggtattcggcggagggaccaagctgaccgtcctaggc

SEQ ID NO: 54 - protein, 8 aa, Artificial Sequence
  Anti-anterior gradient protein 2 homolog antibody CPR02-HCDR3
GAGPTFDY

SEQ ID NO: 55 - protein, 6 aa, Artificial Sequence
  CPR02-HCDR1
SSYAMS

SEQ ID NO: 56 - protein, 17 aa, Artificial Sequence
  CPR02-HCDR2
AISGSGGSTYYADSVKG

SEQ ID NO: 57 - protein, 11 aa, Artificial Sequence
  CPR02 LCDR1
QGDSLRSYYAS

SEQ ID NO: 58 - protein, 7 aa, Artificial Sequence
  CPR02 LCDR2
GKNNRPS

SEQ ID NO: 59 - protein, 11 aa, Artificial Sequence
  CPR02 LCDR3
NSSPPTRAVVV

SEQ ID NO: 60 - protein, 117 aa, Artificial Sequence
  CPR02-VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGAGPTFDYWGQGTLVTVSS

SEQ ID NO: 61 - protein, 109 aa, Artificial Sequence
  CPR02-VL
SSELTQDPAVSVALGQTVRITCQGDSLRSYYASWYQQKPGQAPVLVIYGKNNRPSGIPDRFSGSSSGNTASLTITGAQAEDEADYYCNSSPPTRAVVVFGGGTKLTVLG

SEQ ID NO: 62 - protein, 240 aa, Artificial Sequence
  CPR02 full-length scFv sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGAGPTFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSPPTRAVVVFGGGTKLTVLG

SEQ ID NO: 63 - DNA, 351 bp, Artificial Sequence
  CPR02-VH
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaaggtgctgggcctacttttgactactggggccagggaaccctggtcaccgtctcgagt

SEQ ID NO: 64 - DNA, 327 bp, Artificial Sequence
  CPR02-VL
tcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctccgcccacgcgtgctgttgtggtattcggcggagggaccaagctgaccgtcctaggc

SEQ ID NO: 65 - DNA, 720 bp, Artificial Sequence
  CPR02 Full scFv
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaaggtgctgggcctacttttgactactggggccagggaaccctggtcaccgtctcgagtggtggaggcggttcaggcggaggtggctctggcggtggcggatcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctccgcccacgcgtgctgttgtggtattcggcggagggaccaagctgaccgtcctaggc

SEQ ID NO: 66 - protein, 135 aa, Artificial Sequence
  Residues 41-175 of Agr2 from homo sapiens
PQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 67 - protein, 323 aa, Artificial Sequence; identical to SEQ ID NO: 16
  Galectin-4 (Uniprot P56470, residues 1-323) from homo sapiens

SEQ ID NO: 68 - protein, 9 aa, Artificial Sequence
  Anti-galectin-4 scFv FF04-HCDR3
MVWFELFDY

SEQ ID NO: 69 - protein, 6 aa, Artificial Sequence
  FF04-HCDR1
SSYAMS

SEQ ID NO: 70 - protein, 17 aa, Artificial Sequence
  FF04-HCDR2
AIKGSGGSTYYADSVKG

SEQ ID NO: 71 - protein, 12 aa, Artificial Sequence
  FF04-LCDR1
RASQSVSSSYLA

SEQ ID NO: 72 - protein, 7 aa, Artificial Sequence
  FF04-LCDR2
GASSRAT

SEQ ID NO: 73 - protein, 9 aa, Artificial Sequence
  FF04-LCDR3
QQARGMPLT

SEQ ID NO: 74 - protein, 118 aa, Artificial Sequence
  FF04-VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMVWFELFDYWGQGTLVTVSS

SEQ ID NO: 75 - protein, 108 aa, Artificial Sequence
  FF04-VL
EIVLTQSPGTLSLSPGERATLSCRASQSVSSSYLAWYQQKPGQAPRLLIYGASSRATGIPDRFSGSGSGTDFTLTISRLEPEDFAVYYCQQARGMPLTFGQGTKVEIK

SEQ ID NO: 76 - protein, 240 aa, Artificial Sequence
  FF04 Full-length scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMVWFELFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQARGMPLTFGQGTKVEIK

SEQ ID NO: 77 - DNA, 354 bp, Artificial Sequence
  FF04-VH
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaaatggtttggtttgagctgtttgactactggggccagggaaccctggtcaccgtctcgagt

SEQ ID NO: 78 - DNA, 324 bp, Artificial Sequence
  FF04-VL
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaggcgcggggtatgccgcttacgttcggccaagggaccaaggtggaaatcaaa

SEQ ID NO: 79 - DNA, 720 bp, Artificial Sequence
  FF04 Full-length scFv
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaaatggtttggtttgagctgtttgactactggggccagggaaccctggtcaccgtctcgagtggtggaggcggttcaggcggaggtggctctggcggtggcggagaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaggcgcggggtatgccgcttacgttcggccaagggaccaaggtggaaatcaaa

SEQ ID NO: 80 - protein, 8 aa, Artificial Sequence
  Anti-galectin-4 scFv FF05-HCDR3
TRRLKFDY

SEQ ID NO: 81 - protein, 6 aa, Artificial Sequence
  FF05-HCDR1
SSYAMS

SEQ ID NO: 82 - protein, 17 aa, Artificial Sequence
  FF05-HCDR2
AISGSGGSTYYADSVKG

SEQ ID NO: 83 - protein, 11 aa, Artificial Sequence
  FF05-LCDR1
QGDSLRSYYAS

SEQ ID NO: 84 - protein, 7 aa, Artificial Sequence
  FF05-LCDR2
GKNNRPS

SEQ ID NO: 85 - protein, 11 aa, Artificial Sequence
  FF05-LCDR3
NSSAWNYPIVV

SEQ ID NO: 86 - protein, 117 aa, Artificial Sequence
  FF05-VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTRRLKFDYWGQGTLVTVSS

SEQ ID NO: 87 - protein, 109 aa, Artificial Sequence
  FF05-VL
SSELTQDPAVSVALGQTVRITCQGDSLRSYYASWYQQKPGQAPVLVIYGKNNRPSGIPDRFSGSSSGNTASLTITGAQAEDEADYYCNSSAWNYPIVVFGGGTKLTVLG

SEQ ID NO: 88 - protein, 240 aa, Artificial Sequence
  FF05-Full-length scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTRRLKFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSAWNYPIVVFGGGTKLTVLG

SEQ ID NO: 89 - DNA, 351 bp, Artificial Sequence
  FF05-VH
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaaacgcggcgtctgaagtttgactactggggccagggaaccctggtcaccgtctcgagt

SEQ ID NO: 90 - DNA, 327 bp, Artificial Sequence
  FF05-VL
tcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctgcttggaattatcccattgtggtattcggcggagggaccaagctgaccgtcctaggc

SEQ ID NO: 91 - DNA, 720 bp, Artificial Sequence
  FF05-Full-length scFv
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaaacgcggcgtctgaagtttgactactggggccagggaaccctggtcaccgtctcgagtggtggaggcggttcaggcggaggtggctctggcggtggcggatcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctgcttggaattatcccattgtggtattcggcggagggaccaagctgaccgtcctaggc

SEQ ID NO: 92 - protein, 132 aa, Artificial Sequence; identical to SEQ ID NO: 14
  galectin-2 from homo sapiens

SEQ ID NO: 93 - protein, 9 aa, Artificial Sequence
  Anti-galectin-2 scFv FF06-HCDR3
TRPFRVFDY

SEQ ID NO: 94 - protein, 6 aa, Artificial Sequence
  FF06-HCDR1
SSYAMS

SEQ ID NO: 95 - protein, 17 aa, Artificial Sequence
  FF06-HCDR2
AISGSGGSTYYADSVKG

SEQ ID NO: 96 - protein, 11 aa, Artificial Sequence
  FF06-LCDR1
QGDSLRSYYAS

SEQ ID NO: 97 - protein, 7 aa, Artificial Sequence
  FF06-LCDR2
GKNNRPS

SEQ ID NO: 98 - protein, 11 aa, Artificial Sequence
  FF06-LCDR3
NSSRSRRPAVV

SEQ ID NO: 99 - protein, 118 aa, Artificial Sequence
  FF06-VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTRPFRVFDYWGQGTLVTVSS

SEQ ID NO: 100 - protein, 109 aa, Artificial Sequence
  FF06-VL
SSELTQDPAVSVALGQTVRITCQGDSLRSYYASWYQQKPGQAPVLVIYGKNNRPSGIPDRFSGSSSGNTASLTITGAQAEDEADYYCNSSRSRRPAVVFGGGTKLTVLG

SEQ ID NO: 101 - protein, 241 aa, Artificial Sequence
  FF06 Full-length scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTRPFRVFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSRSRRPAVVFGGGTKLTVLG

SEQ ID NO: 102 - DNA, 354 bp, Artificial Sequence
  FF06-VH
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaaactcggccgtttagggtttttgactactggggccagggaaccctggtcaccgtctcgagt

SEQ ID NO: 103 - DNA, 327 bp, Artificial Sequence
  FF06-VL
tcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctcggtcgcggcgtcccgctgtggtattcggcggagggaccaagctgaccgtcctaggc

SEQ ID NO: 104 - DNA, 723 bp, Artificial Sequence
  FF06 Full-length scFv
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaagacacggccgtatattactgtgcgaaaactcggccgtttagggtttttgactactggggccagggaaccctggtcaccgtctcgagtggtggaggcggttcaggcggaggtggctctggcggtggcggatcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctcggtcgcggcgtcccgctgtggtattcggcggagggaccaagctgaccgtcctaggc

SEQ ID NO: 105 - protein, 8 aa, Artificial Sequence
  Anti-human cadherin-17 antibody AV17.3 HCDR3
MSIRGFDY

SEQ ID NO: 106 - protein, 6 aa, Artificial Sequence
  AV17.3 HCDR1
SSYAMS

SEQ ID NO: 107 - protein, 17 aa, Artificial Sequence
  AV17.3 HCDR2
AIKGSGGSTYYADSVKG

SEQ ID NO: 108 - protein, 11 aa, Artificial Sequence
  AV17.3 LCDR1
QGDSLRSYYAS

SEQ ID NO: 109 - protein, 7 aa, Artificial Sequence
  AV17.3 LCDR2
GKNNRPS

SEQ ID NO: 110 - protein, 10 aa, Artificial Sequence
  AV17.3 LCDR3
NSSRGVRPLV

SEQ ID NO: 111 - protein, 117 aa, Artificial Sequence
  AV17.3 VH
EVQLLESGGGLVQPGGSLRLSCAASGFTFSSYAMSWVRQAPGKGLEWVSAIKGSGGSTYYADSVKGRFTISRDNSKNTLYLQMNSLRAEDTAVYYCAKMSIRGFDYWGQGTLVTVSS

SEQ ID NO: 112 - protein, 109 aa, Artificial Sequence
  AV17.3 VL
SSELTQDPAVSVALGQTVRITCQGDSLRSYYASWYQQKPGQAPVLVIYGKNNRPSGIPDRFSGSSSGNTASLTITGAQAEDEADYYCNSSRGVRPLVVFGGGTKLTVLG

SEQ ID NO: 113 - protein, 240 aa, Artificial Sequence
  AV17.3 full-length scFv
EVQLLESGGGLVQPGGSLRLSCAASGFTFSSYAMSWVRQAPGKGLEWVSAIKGSGGSTYYADSVKGRFTISRDNSKNTLYLQMNSLRAEDTAVYYCAKMSIRGFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSRGVRPLVVFGGGTKLTVLG

SEQ ID NO: 114 - DNA, 720 bp, Artificial Sequence
  AV17.3 full-length scFv
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattaagggtagtggtggtagcacatactacgcagactccgtgaagggccggttcaccatctccagagacaattccaagaacacgctgtatctgcaaatgaacagcctgagagccgaagacacggccgtatattactgtgcgaaaatgtcgattaggggttttgactactggggccagggaaccctggtcaccgtctcgagtggtggaggcggttcaggcggaggtggctctggcggtggcggatcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctaggggggttcgtcccttggtggtattcggcggagggaccaagctgaccgtcctaggc

SEQ ID NO: 115 - DNA, 351 bp, Artificial Sequence
  AV17.3 VH
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattaagggtagtggtggtagcacatactacgcagactccgtgaagggccggttcaccatctccagagacaattccaagaacacgctgtatctgcaaatgaacagcctgagagccgaagacacggccgtatattactgtgcgaaaatgtcgattaggggttttgactactggggccagggaaccctggtcaccgtctcgagt

SEQ ID NO: 116 - DNA, 327 bp, Artificial Sequence
  AV17.3 VL
tcgtctgagctgactcaggaccctgctgtgtctgtggccttgggacagacagtcaggatcacatgccaaggagacagtctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctaggggggttcgtcccttggtggtattcggcggagggaccaagctgaccgtcctaggc

(19) United States
(12) Patent Application Publication
(10) Pub. No.: US 2019/0309060 A1
(43) Pub. Date: Oct. 10, 2019

(54) INTESTINAL ANTIGENS FOR PHARMACODELIVERY APPLICATIONS

(71) Applicant: PHILOGEN S.P.A., Siena (IT)

(72) Inventors: Alessandra VILLA, Zurich (CH); Catherine PEMBERTON ROSS, Brugg (CH); Francesca PRETTO, Zurich (CH); Filippa FLEETWOOD, Otelfingen (CH); Tim FUGMANN, Adlikon bei Regensdorf (CH)

(21) Appl. No.: 16/070,032

(22) PCT Filed: Jan. 13, 2017
(86) PCT No.: PCT/EP2017/050725

§ 371 Date: Jul. 13, 2018

(30) Foreign Application Priority Data
Jan. 15, 2016 (GB) 1600801.3
Jul. 8, 2016 (GB) 1611958.8

(51) Int. Cl. C07K 16/28; C07K 16/40; A61P 1/00

(52) U.S. Cl. CPC C07K 16/28; C07K 16/40; A61P 1/00; C07K 2317/24; C07K 2317/33; C07K 2317/622; C07K 2317/92; C07K 2317/21

(57) ABSTRACT

The present invention relates to the treatment of intestinal diseases and disorders, such as inflammatory bowel disease (IBD). The invention involves antibodies and the use of antibodies which bind antigens that have been shown to be over-expressed in the intestine compared to other organs, including antigens cadherin-17, anterior gradient protein 2 homolog, galectin-4, and galectin-2. The antibodies may be conjugated to anti-inflammatory or immunosuppressive agents, as required.

### FIELD OF THE INVENTION

The present invention relates to the treatment of intestinal diseases and disorders, such as inflammatory bowel disease (IBD). The invention involves the use of antibodies which bind antigens that have been shown to be over-expressed in the intestine compared to other organs, including antigens cadherin-17, anterior gradient protein 2 homolog, galectin-4, and galectin-2. The antibodies may be conjugated to anti-inflammatory or immunosuppressive agents, as required. Antibodies which bind to cadherin-17, anterior gradient protein 2 homolog, galectin-4, or galectin-2 are also provided.

### BACKGROUND TO THE INVENTION

The antibody-based pharmacodelivery of cytokines “immunocytokines” to sites of disease as a new therapeutic modality is gaining momentum with new lead products entering clinical trials (Pasche and Neri, Drug Discovery Today, 2012, 17, 583-590).

At present, there are more than 50 immunocytokine products, which have been investigated for therapeutic applications in oncology. By contrast, few studies have reported the development and use of immunocytokines for the treatment of chronic inflammatory conditions. Among the different anti-inflammatory cytokines tested, Interleukin-10-based immunocytokines have shown promise for the treatment of mouse models of rheumatoid arthritis (WO2007/128563, WO2009/056268) and of inflammatory bowel disorders (IBD) (WO2014/055073), while Interleukin-4-based immunocytokines have shown promise for the treatment of mouse model of endometriosis and of psoriasis (WO2014/173570).

Other than IL-4 and IL-10, other cytokines of particular pharmaceutical interest include the p40 subunit of IL-12, IL-22, IL-27, IL-33, IL-35 and GM-CSF.

Interleukin-4 (IL-4) has been shown to suppress the production of the pro-inflammatory cytokines tumor necrosis factor (TNF)-α and interleukin-1β (IL-1β) in lipopolysaccharide (LPS)-activated human monocytes [1]. In addition, IL-4 further antagonizes IL-1 action by inducing the production of the IL-1 receptor antagonist (IL-1Ra; [2]) and a decoy type II receptor [3]. Furthermore, IL-4 has been described as inducing protective effects against cartilage and bone erosion [4-6]. IL-4 also promotes alternative activation of macrophages into M2 macrophages, which leads to IL-1Ra, IL-10 and TGFβ secretion and finally to amelioration of inflammation, wound healing and tissue repair [7, 8]. Beneficial effects of IL-4 have been observed in several pathological inflammatory conditions, for example, psoriasis [9], and rheumatoid arthritis [6, 10].

Interleukin-10 (IL-10) has multiple anti-inflammatory effects, which are essential in maintaining immune system homeostasis. In general, IL-10 activity increases tolerance in adaptive immunity while it inhibits all activities promoting inflammation including a specific cellular immune response [11]. Processes involved in these anti-inflammatory effects include downregulation of products involved in inflammation (e.g. TH1 cytokines, MHC class II antigens, co-stimulatory molecules on macrophages) and inhibition of expression of pro-inflammatory cytokines (TNFα, IL1β, IL-12 and IFNγ, [12-15]) accompanied by enhancement of production of anti-inflammatory mediators (e.g. IL1-Ra, soluble TNF receptors [16-20]). Early clinical phase II trials revealed a beneficial effect of IL-10 administration in inflammatory conditions, such as psoriasis, rheumatoid arthritis and inflammatory bowel disease [11].

The p40 subunit (IL12p40) of the heterodimeric cytokines interleukin-12 (IL-12) and interleukin-23 (IL23) can form a homodimer, also known as p80, which antagonizes the pro-inflammatory activity of IL-12 and IL-23 by binding their receptors and thereby blocking their function [21-24]. A recent study indicated that DSS-induced colitis in mice could be attenuated by the delivery of IL12p40 by a recombinant adenovirus or mesenchymal stem cells [25].

Biological effects of interleukin-22 (IL-22) contribute to pathogen defence (activation of anti-microbial pathways), wound healing and tissue protection, regeneration and reorganization through increased proliferation and migration [26]. A protective role of IL-22 in colitis has been reported whilst comparing disease severity in wild-type mice and IL-22-deficient mice. A beneficial role of IL-22 was observed both in DSS-induced colitis, as well as in TH1- and TH2-mediated colitis [26, 27]. Similarly, administration of an IL-22-blocking antibody worsened colitis symptoms [28].

Interleukin-27 (IL-27) can induce the secretion of IL-10 and limits inflammatory responses in the context of infection. It can further limit IL-2 production and reverses the IL-23 mediated lineage commitment of Th17 cells [29]. Administration of IL-27 to mice with experimental colitis attenuated the disease through the suppression of the Th17 development [30].

Interleukin-35 (IL-35) is produced by resting and activated regulatory T cells and is required for their maximal suppressive capacity. By arresting the cell-cycle in the G1 phase IL-35 suppresses naïve and effector T cell proliferation. IL-35 further enhances regulatory T cell proliferation and induces the development of a specific subset of immunosuppressive T cells (iTr35 cells, [31-33]). IL-35 has been shown to have a potent anti-inflammatory effect in mice with T cell dependent colitis. Ebi3 deficient mice demonstrated increased pathological features in this model, whereas ectopical expression of single chain IL-35 protein significantly improved inflammatory conditions [34].

Granulocyte-macrophage colony-stimulating factor (GM-CSF) stimulates stem cells to produce granulocytes and monocytes and is involved in host defence against bacteria and fungi [35, 36]. Recombinant GM-CSF is therapeutically used to prevent neutropenia after chemotherapy [37]. A randomized, placebo-controlled clinical trial revealed that patients with Crohn's disease significantly benefit from GM-CSF administration [38].

Under non-inflamed conditions, interleukin-33 (IL-33) acts as an intracellular nuclear factor. During inflammation, however, it is released from cells and acts as an alarmin. It is mainly produced by myofibroblasts, endothelial and epithelial cells and in barrier surfaces with direct contact to the external environment, where it is thought to exert tissue remodeling and anti-inflammatory effects [39-41]. IL-33 appears to enhance intestinal inflammation in some models and reduce it in others. In the acute phase of tissue damage IL-33 worsens disease [39, 42, 43], but seems to enhance wound healing and tissue repair during the recovery phase [39, 44]. It may amplify the innate immune response in the beginning of inflammatory processes, but its part in chronic processes remains to be clarified. Initial research suggests the IL-33 could pose a viable asset in the treatment of IBD.

The ED-A domain of fibronectin has been described to be an excellent target for pharmacodelivery of cytokines in IBD (Bootz et al., 2015, Inflamm Bowel Dis, 21, 1908-1917), however there is a continuing need for additional target antigens for antibody-mediated delivery of therapeutic agents, such as cytokines, to site of intestinal diseases and disorders.

### SUMMARY OF INVENTION

The identification of antigens for antibody-based pharmacodelivery is not trivial as the ideal candidate antigen should be abundant in the region of disease, but absent or expressed only at low level in other tissues. To solve this problem, the present inventors performed a complex proteomic analysis in mice in which more than 2,000 proteins were screened and their expression in different organs analyzed. As a result of this analysis, the present inventors identified thirteen proteins that were preferentially expressed in the intestine of mice compared with other tissues. The thirteen proteins were: galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, and sulfate transporter.

It is expected that the corresponding human proteins are similarly overexpressed in human intestine relative to other human organs. This makes these proteins promising targets for antibody-mediated pharmacodelivery of therapeutic agents, such as anti-inflammatory molecules, to the human intestine to treat intestinal diseases and disorders. The expression of galectin-2 and anterior gradient protein 2 homolog in the intestine of humans was confirmed by immunofluorescence analysis, as described below.

The present invention thus provides an antibody molecule, or fragment thereof, for use in a method of treating an intestinal disease or disorder in a patient,
wherein the antibody molecule, or fragment, binds galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter.

Also provided is a method of treating an intestinal disease or disorder in a patient, the method comprising administering a therapeutically effective amount of a medicament comprising an antibody molecule, or fragment thereof, to the patient,
wherein the antibody molecule, or fragment, binds galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter.

The present invention further provides the use of an antibody molecule, or fragment thereof, for the manufacture of a medicament for the treatment of an intestinal disease or disorder in a patient,
wherein the antibody molecule, or fragment, binds galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter.

As mentioned above, the thirteen proteins identified by the present inventors are expected to be overexpressed in the human intestine. This means that antibodies which bind these proteins can be used to deliver therapeutic molecules, such anti-inflammatory or immunosuppressive agents, to the intestine to treat intestinal diseases or disorders. Delivery to sites of an intestinal disease or disorder in a patient in the context of the present invention may thus refer to delivery to the intestine of the patient, rather than specifically to diseased tissue.

The expression of galectin-2, anterior gradient protein 2 homolog and cadherin-17 in the intestine of humans having ulcerative colitis was further confirmed by immunofluorescence analysis as described herein.

Accordingly, the present invention provides an antibody molecule, or fragment thereof, for use in a method of delivering a therapeutic molecule to sites of an intestinal disease or disorder in a patient,
wherein the antibody molecule, or fragment, binds galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter, and wherein the antibody molecule or fragment is conjugated to the therapeutic molecule.

The invention also provides a method of delivering a therapeutic molecule to sites of an intestinal disease or disorder in a patient, the method comprising administering an antibody molecule, or fragment thereof, to the patient,
wherein the antibody molecule, or fragment, binds galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter, and wherein the antibody molecule or fragment is conjugated to the therapeutic molecule.

The use of an antibody molecule, or fragment thereof, for the manufacture of a medicament for the delivery of a therapeutic molecule to sites of an intestinal disease or disorder in a patient,
wherein the antibody molecule, or fragment, binds galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter, and wherein the antibody molecule or fragment is conjugated to the therapeutic molecule, is similarly provided.

The present invention also relates to antibody molecules, and fragments thereof, which bind to cadherin-17, anterior gradient protein 2 homolog, galectin-4, or galectin-2. These antibody molecules are suitable for use in a method of treating an intestinal disease or disorder in a patient, or delivering an anti-inflammatory molecule to sites of an intestinal disease or disorder in a patient, as disclosed herein.

The present invention thus also relates to an antibody molecule, or fragment thereof, which binds cadherin-17. Preferably, the antibody molecule, or fragment thereof, binds domain 1-2 of cadherin-17. The cadherin-17 is preferably human cadherin-17.

In a preferred embodiment, the antibody molecule, or fragment thereof, comprises one or more of the complementarity determining regions (CDRs), variable heavy chain (VH), and/or variable light chain (VL) sequences of the anti-cadherin-17 antibodies AV17, AV17.2 or AV17.3 disclosed herein.

Specifically, the antibody molecule, or fragment thereof, preferably comprises the HCDR3 of the AV17 antibody molecule, wherein the HCDR3 comprises, has, or consists of the sequence set forth in SEQ ID NO: 28, or the sequence set forth in SEQ ID NO: 28 with three or fewer amino acid substitutions, deletions, or insertions. In addition, the antibody molecule, or fragment thereof, may comprise the HCDR1, HCDR2, LCDR1, LCDR2, and/or LCDR3 of the AV17 antibody molecule, wherein the HCDR1, HCDR2, LCDR1, LCDR2, and/or LCDR3 comprises, has, or consists of the sequence set forth in SEQ ID NOs 29-33, or the sequence set forth in SEQ ID NOs 29-33 with three or fewer amino acid substitutions, deletions, or insertions. For example, the antibody molecule, or fragment thereof, may comprise the VH domain and/or VL domain of the AV17 antibody molecule, wherein the VH domain and/or VL domain comprises, has, or consists of the sequence set forth in SEQ ID NOs 34 and 35, respectively, or the sequence set forth in SEQ ID NOs 34 and 35, respectively, with ten or fewer amino acid substitutions, deletions, or insertions. The VH and VL domains of the antibody molecule, or fragment thereof, may be joined through a peptide linker, preferably a peptide linker which has, comprises, or consists of the amino acid sequence set forth in SEQ ID NO: 40. The amino acid sequence of the full-length AV17 scFv is set forth in SEQ ID NO: 36. Thus, the antibody molecule, or fragment thereof, preferably comprises, or has, the amino acid sequence set forth in SEQ ID NO: 36, or the sequence set forth in SEQ ID NO: 36 with twenty or fewer amino acid substitutions, deletions, or insertions.

Alternatively, the antibody molecule, or fragment thereof, may preferably comprise the HCDR3 of the AV17.2 antibody molecule, wherein the HCDR3 comprises, has, or consists of the sequence set forth in SEQ ID NO: 42, or the sequence set forth in SEQ ID NO: 42 with three or fewer amino acid substitutions, deletions, or insertions. In addition, the antibody molecule, or fragment thereof, may comprise the HCDR1, HCDR2, LCDR1, LCDR2, and/or LCDR3 of the AV17.2 antibody molecule, wherein the HCDR1, HCDR2, LCDR1, LCDR2, and/or LCDR3 comprises, has, or consists of the sequence set forth in SEQ ID NOs 43-47, or the sequence set forth in SEQ ID NOs 43-47 with three or fewer amino acid substitutions, deletions, or insertions. For example, the antibody molecule, or fragment thereof, may comprise the VH domain and/or VL domain of the AV17.2 antibody molecule, wherein the VH domain and/or VL domain comprises, has, or consists of the sequence set forth in SEQ ID NOs 48 and 49, respectively, or the sequence set forth in SEQ ID NOs 48 and 49, respectively, with ten or fewer amino acid substitutions, deletions, or insertions. The VH and VL domains of the antibody molecule, or fragment thereof, may be joined through a peptide linker, preferably a peptide linker which has, comprises, or consists of the amino acid sequence set forth in SEQ ID NO: 40. The amino acid sequence of the full-length AV17.2 scFv is set forth in SEQ ID NO: 50. Thus, the antibody molecule, or fragment thereof, preferably comprises, or has, the amino acid sequence set forth in SEQ ID NO: 50 or the sequence set forth in SEQ ID NO: 50 with twenty or fewer amino acid substitutions, deletions, or insertions.

As a further alternative, the antibody molecule, or fragment thereof, may preferably comprise the HCDR3 of the AV17.3 antibody molecule, wherein the HCDR3 comprises, has, or consists of the sequence set forth in SEQ ID NO: 105, or the sequence set forth in SEQ ID NO: 105 with three or fewer amino acid substitutions, deletions, or insertions. In addition, the antibody molecule, or fragment thereof, may comprise the HCDR1, HCDR2, LCDR1, LCDR2, and/or LCDR3 of the AV17.3 antibody molecule, wherein the HCDR1, HCDR2, LCDR1, LCDR2, and/or LCDR3 comprises, has, or consists of the sequence set forth in SEQ ID NOs 106-110, or the sequence set forth in SEQ ID NOs 106-110 with three or fewer amino acid substitutions, deletions, or insertions. For example, the antibody molecule, or fragment thereof, may comprise the VH domain and/or VL domain of the AV17.3 antibody molecule, wherein the VH domain and/or VL domain comprises, has, or consists of the sequence set forth in SEQ ID NOs 111 and 112, respectively, or the sequence set forth in SEQ ID NOs 111 and 112, respectively, with ten or fewer amino acid substitutions, deletions, or insertions. The VH and VL domains of the antibody molecule, or fragment thereof, may be joined through a peptide linker, preferably a peptide linker which has, comprises, or consists of the amino acid sequence set forth in SEQ ID NO: 40. The amino acid sequence of the full-length AV17.3 scFv is set forth in SEQ ID NO: 113. Thus, the antibody molecule, or fragment thereof, preferably comprises, or has, the amino acid sequence set forth in SEQ ID NO: 113 or the sequence set forth in SEQ ID NO: 113 with twenty or fewer amino acid substitutions, deletions, or insertions.

The present invention also relates to an antibody molecule, or fragment thereof, which binds anterior gradient protein 2 homolog (Agr2). Preferably, the antibody molecule, or fragment thereof, binds amino acids 41-175 of anterior gradient protein 2 homolog. The anterior gradient protein 2 homolog is preferably human anterior gradient protein 2 homolog.

In a preferred embodiment, the antibody molecule, or fragment thereof, comprises one or more of the CDRs, VH, and/or VL domain sequences of the anti-Agr2 antibody CPR02 disclosed herein.

Specifically, the antibody molecule, or fragment thereof, preferably comprises the HCDR3 of the CPR02 antibody molecule, wherein the HCDR3 comprises, has, or consists of the sequence set forth in SEQ ID NO: 54, or the sequence set forth in SEQ ID NO: 54 with three or fewer amino acid substitutions, deletions, or insertions. In addition, the antibody molecule, or fragment thereof, may comprise the HCDR1, HCDR2, LCDR1, LCDR2, and/or LCDR3 of the CPR02 antibody molecule, wherein the HCDR1, HCDR2, LCDR1, LCDR2, and/or LCDR3 comprises, has, or consists of the sequence set forth in SEQ ID Nos 55-59, or the sequence set forth in SEQ ID NOs 55-59 with three or fewer amino acid substitutions, deletions, or insertions. For example, the antibody molecule may comprise the VH domain and/or VL domain of the CPR02 antibody, wherein the VH domain and/or VL domain comprises, has, or consists of the sequence set forth in SEQ ID NOs 60 and 61, respectively, or the sequence set forth in SEQ ID NOs 60 and 61, respectively, with ten or fewer amino acid substitutions, deletions, or insertions. The VH and VL domains of the antibody molecule, or fragment thereof, may be joined through a peptide linker, preferably a peptide linker which has, comprises, or consists of the amino acid sequence set forth in SEQ ID NO: 40. The amino acid sequence of the full-length CPR02 scFv is set forth in SEQ ID NO: 62. Thus, the antibody molecule, or fragment thereof, preferably comprises, or has, the amino acid sequence set forth in SEQ ID NO: 62, or the sequence set forth in SEQ ID NO: 62 with twenty or fewer amino acid substitutions, deletions, or insertions.

The present invention also relates to an antibody molecule, or fragment thereof, which binds galectin-4. The galectin-4 is preferably human galectin-4.

In a preferred embodiment, the antibody molecule, or fragment thereof, comprises one or more of the CDRs, VH, and/or VL domain sequences of the anti-galectin-4 antibodies FF04 and FF05 disclosed herein.

Specifically, the antibody molecule, or fragment thereof, preferably comprises the HCDR3 of the FF04 antibody molecule, wherein the HCDR3 comprises, has, or consists of the sequence set forth in SEQ ID NO: 68, or the sequence set forth in SEQ ID NO: 68 with three or fewer amino acid substitutions, deletions, or insertions. In addition, the antibody molecule, or fragment thereof, may comprise the HCDR1, HCDR2, LCDR1, LCDR2, and/or LCDR3 of the FF04 antibody molecule, wherein the HCDR1, HCDR2, LCDR1, LCDR2, and/or LCDR3 comprises, has, or consists of the sequence set forth in SEQ ID NOs 69-73, or the sequence set forth in SEQ ID NOs 69-73 with three or fewer amino acid substitutions, deletions, or insertions. For example, the antibody molecule, or fragment thereof, may comprise the VH domain and/or VL domain of the FF04 antibody, wherein the VH domain and/or VL domain comprises, has, or consists of the sequence set forth in SEQ ID NOs 74 and 75, respectively, or the sequence set forth in SEQ ID NOs 74 and 75, respectively, with ten or fewer amino acid substitutions, deletions, or insertions. The VH and VL domains of the antibody molecule, or fragment thereof, may be joined through a peptide linker, preferably a peptide linker which has, comprises, or consists of the amino acid sequence set forth in SEQ ID NO: 40. The amino acid sequence of the full-length FF04 scFv is set forth in SEQ ID NO: 76. Thus, the antibody molecule, or fragment thereof, preferably comprises, or has, the amino acid sequence set forth in SEQ ID NO: 76, or the sequence set forth in SEQ ID NO: 76 with twenty or fewer amino acid substitutions, deletions, or insertions

Alternatively, the antibody molecule, or fragment thereof, may preferably comprise the HCDR3 of the FF05 antibody molecule, wherein the HCDR3 comprises, has, or consists of the sequence set forth in SEQ ID NO: 80, or the sequence set forth in SEQ ID NO: 80 with three or fewer amino acid substitutions, deletions, or insertions. In addition, the antibody molecule, or fragment thereof, may comprise the HCDR1, HCDR2, LCDR1, LCDR2, and/or LCDR3 sequences of the FF05 antibody, wherein the HCDR1, HCDR2, LCDR1, LCDR2, and/or LCDR3 comprises, has, or consists of the sequence set forth in SEQ ID NOs 81-85, or the sequence set forth in SEQ ID NOs 81-85 with three or fewer amino acid substitutions, deletions, or insertions. For example, the antibody molecule, or fragment thereof, may comprise the VH domain and/or VL domain of the FF05 antibody, wherein the VH domain and/or VL domain comprises, has, or consists of the sequence set forth in SEQ ID NOs 86 and 87, respectively, or the sequence set forth in SEQ ID NOs 86 and 87, respectively, with ten or fewer amino acid substitutions, deletions, or insertions. The VH and VL domains of the antibody molecule, or fragment thereof, may be joined through a peptide linker, preferably a peptide linker which has, comprises, or consists of the amino acid sequence set forth in SEQ ID NO: 40. The amino acid sequence of the full-length FF05 scFv is set forth in SEQ ID NO: 88. Thus, the antibody molecule, or fragment thereof, preferably comprises, or has, the amino acid sequence set forth in SEQ ID NO: 88, or the sequence set forth in SEQ ID NO: 88 with twenty or fewer amino acid substitutions, deletions, or insertions.

The present invention further relates to an antibody molecule, or fragment thereof, that binds galectin-2. The galectin-2 is preferably human galectin-2.

In a preferred embodiment, the antibody molecule, or fragment thereof, comprises one or more of the complementarity determining regions (CDRs), variable heavy chain (VH), and/or variable light chain (VL) sequences of anti-galectin-2 antibody FF06 disclosed herein.

Specifically, the antibody molecule, or fragment thereof, preferably comprises the HCDR3 of the FF06 antibody molecule, wherein the HCDR3 comprises, has, or consists of the sequence set forth in SEQ ID NO: 93, or the sequence set forth in SEQ ID NO: 93 with three or fewer amino acid substitutions, deletions, or insertions. In addition, the antibody molecule, or fragment thereof, may comprise the HCDR1, HCDR2, LCDR1, LCDR2, and/or LCDR3 of the FF06 antibody, wherein the HCDR1, HCDR2, LCDR1, LCDR2, and/or LCDR3 comprises, has, or consists of the sequence set forth in SEQ ID NOs 94-98, or the sequence set forth in SEQ ID NOs 94-98 with three or fewer amino acid substitutions, deletions, or insertions. For example, the antibody molecule, or fragment thereof, may comprise the VH domain and/or VL domain of the anti-galectin-2 FF06 antibody molecule, wherein the VH domain and/or VL domain comprises, has, or consists of the sequence set forth in SEQ ID NOs 99 and 100, or the sequence set forth in SEQ ID NOs 99 and 100, respectively, with ten or fewer amino acid substitutions, deletions, or insertions. The VH and VL domains of the antibody molecule, or fragment thereof, may be joined through a peptide linker, preferably a peptide linker which has, comprises, or consists of the amino acid sequence set forth in SEQ ID NO: 40. The amino acid sequence of the full-length FF06 scFv is set forth in SEQ ID NO: 101. Thus, the antibody molecule, or fragment thereof, preferably comprises, or has, the amino acid sequence set forth in SEQ ID NO: 101, or the sequence set forth in SEQ ID NO: 101 with twenty or fewer amino acid substitutions, deletions, or insertions.

As mentioned herein, an antibody molecule, or fragment thereof, of the invention, or for use in the invention, may comprise a HCDR3 sequence as described herein with three or fewer amino acid substitutions, deletions, or insertions. For example, an antibody molecule, or fragment thereof, of the invention, or for use in the invention, may comprise a HCDR3 sequence as described herein with two or fewer, or one, amino acid substitution(s), deletion(s), or insertion(s). As with regard to the HCDR3 sequences, an antibody molecule, or fragment thereof, of the invention, or for use in the invention, may comprise a HCDR1, HCDR2, LCDR1, LCDR2, and/or LCDR3 sequence, as described herein, with three or fewer, two or fewer, or one amino acid substitution(s), deletion(s), or insertion(s). Similarly, an antibody molecule, or fragment thereof, of the invention, or for use in the invention, may comprise a VH and/or VL domain sequence as described herein with ten or fewer, e.g. nine or fewer, eight or fewer, seven or fewer, six or fewer, five or fewer, four or fewer, three or fewer, two or fewer, or one amino acid substitution(s), deletion(s), or insertion(s). An antibody molecule, or fragment thereof, of the invention, or for use in the invention, may be or comprise an scFv, wherein the scFv comprises a sequence as described herein with twenty or fewer, e.g. fifteen or fewer, ten or fewer, nine or fewer, eight or fewer, seven or fewer, six or fewer, five or fewer, four or fewer, three or fewer, two or fewer, or one amino acid substitution(s), deletion(s), or insertion(s). Where the VH and/or VL domain, or scFv sequence are concerned, the amino acid substitution(s), deletion(s), or insertion(s) may be in the framework regions of the VH and/or VL domain, or scFv.

Where the present application discloses that an antibody HCDR1, HCDR2, HCDR3, LCDR1, LCDR2, LCDR3, VH, VL, and/or scFv has a particular sequence, this may refer to the HCDR1, HCDR2, HCDR3, LCDR1, LCDR2, LCDR3, VH, VL, or scFv comprising, or consisting of, the recited sequence.

An antibody molecule, as referred to herein, may be in any suitable format. Many antibody molecule formats are known in the art and include both complete antibody molecule molecules, such as IgG, as well as antibody molecule fragments, such as a single chain Fv (scFv). The term “antibody molecule” as used herein encompasses both complete antibody molecule molecules and antibody molecule fragments, in particular antigen-binding fragments. Preferably, an antibody molecule comprises a VH domain and a VL domain. In a preferred embodiment, the antibody molecule is or comprises a scFv, is a small immunoprotein (SIP), is a diabody, or is a (complete) IgG molecule.

An antibody molecule of, or for use in, the invention preferably comprises the HCDR1, HCDR2, HCDR3, LCDR1, LCDR2, and/or LCDR3 sequences of an antibody as disclosed or described herein in a framework. The frameworks are preferably human frameworks, specifically human germline frameworks. Thus a VH and/or VL domain framework, as referred to herein, is preferably a human framework, more preferably a human germline framework. For example, the VH domain framework may be DP47 and/or the VL domain framework may be DPL16 or DPK22. The AV17, AV17.2, AV17.3, CPR02, FF05, and FF06 antibodies employed in the present examples, comprise the VH domain human framework germline sequence DP47 and the VL domain human framework germline sequence DPL16, while antibody FF04 employed in the examples, comprises the VH domain human framework germline sequence DP47 and the VL domain human framework germline sequence DPK22.

The antibody molecule, or fragment thereof, is preferably conjugated to a therapeutic molecule, such as an anti-inflammatory or immunosuppressive molecule, more preferably an anti-inflammatory molecule. In a preferred embodiment, the antibody molecule, or fragment, is conjugated to an anti-inflammatory cytokine, such as interleukin-4 (IL-4), interleukin-10 (IL-10), interleukin-12 (IL-12), interleukin-22 (IL-22), interleukin-27 (IL-27), interleukin-35 (IL-35), granulocyte-macrophage colony-stimulating factor (GM-CSF), or interleukin-33 (IL-33).

The invention also provides isolated nucleic acid molecules encoding the antibody molecules, fragments thereof, and conjugates of the invention. The skilled person would have no difficulty in preparing such nucleic acids using methods well-known in the art. An isolated nucleic acid may be used to express the antibody molecule or conjugate of the invention, for example by expression in a bacterial, yeast, insect or mammalian host cell. A preferred host cell is E. coli. The nucleic acid molecule will generally be provided in the form of a recombinant vector for expression. Host cells in vitro comprising such nucleic acids and vectors are part of the invention, as is their use for expressing the antibodies and conjugates of the invention, which may subsequently be purified from cell culture and optionally formulated into a pharmaceutical composition.

An antibody molecule or conjugate of the invention may be provided for example in a pharmaceutical composition, and may be employed for medical use as described herein, either alone or in combination with one or more further therapeutic agents or therapeutically acceptable excipients.

The intestinal disease or disorder, may be inflammatory bowel disease (IBD) or irritable bowel syndrome (IBS), preferable IBD. IBD includes Crohn's disease and ulcerative colitis as well as number of less common diseases.

### BRIEF DESCRIPTION OF THE FIGURES

FIG. 1 shows the results of an ELISA using immobilized human cadherin-17 domain 1-2 expressed in either CHO-S cells or in E. coli BL21 cells and anti-cadherin-17 scFv(AV17) and scFv(AV17.2). The results demonstrate that both scFvs were able to bind human cadherin-17 domain 1-2 produced in CHO-S cells or E. coli BL21 cells.

FIG. 2 shows the affinity constant calculation for scFv(AV17) and scFv(AV17.3). FIG. 2A shows the results of monomeric scFv(AV17) preparation by size-exclusion chromatography (SEC75, Amersham Biosciences). FIG. 2B shows the BiaCore profile of scFv(AV17) on a human cadherin-17 coated CM5-chip (500RUs) and confirms its binding to human cadherin-17. The Kₒₙ, K_{off} and K_{D}, of scFv(AV17) for human cadherin-17 are listed in FIG. 2B. FIG. 2C shows the results of monomeric scFv(AV17.3) preparation by size-exclusion chromatography (SEC75i, Amersham Biosciences). FIG. 2D shows the BiaCore profile of scFv(AV17.3) on a human cadherin-17 coated CM5-chip and FIG. 2E shows the BiaCore profile of scFv(AV17.3) on mouse cadherin-17 coated CM5-chip. These results confirm the binding of AV17.3 antibody to human cadherin-17 and mouse cadherin-17. The Kₒₙ, K_{off} and K_{D}, of scFv(AV17.3) for human cadherin-17 and mouse cadherin-17 are listed in FIGS. 2D and 2E respectively.

FIG. 3 shows the results of an ELISA and BiaCore using the anti-anterior gradient protein 2 homolog (Agr2) scFv antibody. FIG. 3A shows the results of an ELISA using the anti-Agr2 scFv antibody and biotinylated Agr2 antigen. Various concentrations of the scFv antibody were incubated with biotinylated Agr2 (residues 41-175 of recombinant human Agr2) that had been immobilized on avidin-coated maxisorp wells. Bound scFv antibody was detected using the anti-myc antibody 9E10 and an HRP-conjugated anti-murine Fc antibody. The specificity of the antibody-antigen interaction was confirmed by incubating the scFv antibodies with avidin-coated wells as a negative control. The results demonstrate that antibody scFv(CPR02) was able to bind the recombinant human Agr2 protein in an ELISA. FIG. 3B shows the results of the BiaCore analysis of scFv (CPR02) antibody binding to human Agr2. Varying concentrations of the scFv (CPR02) antibody were injected over a CM5 chip surface coated with immobilized human Agr2. The BiaCore analysis confirms that scFv (CPR02) antibody binds to human Agr2.

FIG. 4 shows the results of immunofluorescent staining of colon biopsy samples from a healthy donor with an anti-Agr2 (CPR02) and an anti-Galectin-2 (FF06) biotinylated antibodies in IgG format. Detection of the primary antibody was performed with streptavidin-alexa 488 (Invitrogen). Blood vessels were detected using a mouse anti-human CD31 antibody (eBioscience) followed by detection with an anti-mouse alexa 594 antibody (Invitrogen). Counterstaining for cell nuclei was performed with DAPI (eBioscience). The direct comparison of FF06 and CPR02 to KSF (antibody of irrelevant specificity in human as it recognizes hen egg lysozyme) reveals significant staining surrounding the crypts of the intestinal tissue.

FIG. 5 shows the results of the BiaCore analysis of the anti-galectin-4 scFv antibodies (FF04 and FF05) binding to biotinylated galectin-4. Varying concentrations of the scFv (FF04) and scFv (FF05) antibodies were injected over a CM5 chip surface coated with immobilized Galectin-4. The BiaCore analysis confirms that scFv antibodies FF04 and FF05 bind to human galectin-4.

FIG. 6 shows the results of immunofluorescent staining of LS174T cells with anti-galectin-4 scFv antibodies. Methanol-permeabilized LS174T cells were stained with the anti-galectin-4 scFv antibodies FF04 and FF05. Antibody binding was detected using the anti-myc tag antibody 9E10 and an anti-mouse IgG Alexa594 antibody. Nuclei were stained with DAPI. To confirm the specificity of staining, cells were treated with an irrelevant scFv in combination with the detection antibodies. Pictures were taken using a 40× objective. Both scFv (FF04) and scFv (FF05) showed strong cytoplasmic staining of the LS174T cells.

FIG. 7 shows the results of an ELISA using the anti-galectin-2 scFv antibodies and biotinylated galectin-2. Varying concentrations of the scFv antibodies were incubated with biotinylated galectin-2 that had been immobilized on avidin-coated maxisorp wells. Bound scFv antibody was detected using the anti-myc antibody 9E10 and an HRP-conjugated anti-murine Fc antibody. To confirm the specificity of the binding, one well was incubated with the detection antibodies in the absence of a primary antibody. ScFv (FF06) was able to bind to recombinant human galectin-2 protein in ELISA experiments.

FIG. 8 shows the results of immunofluorescent staining of LS174T cells with anti-galectin-2 scFv antibody. Methanol-permeabilized LS174T cells were stained with the anti-galectin-2 scFv antibody FF06. Antibody binding was detected using the anti-myc tag antibody 9E10 and an anti-mouse IgG Alexa594 antibody. Nuclei were stained with DAPI. To confirm the specificity of staining, cells were treated with an irrelevant scFv in combination with the detection antibodies. Pictures were taken using a 40× objective. ScFv(FF06) showed strong cytoplasmic staining.

FIG. 9 shows the results of immunofluorescent staining of freshly frozen human biopsy samples of Ulcerative Colitis with anti-cadherin 17 (AV17), anti-Agr2 (CPR02) and anti-galectin-2 (FF06) antibodies. Purified antibodies in human IgG1 format were added at the final concentration of 2 μg/ml to the sections. Detection of the primary antibody was performed rabbit anti human-IgG (DAKO) and signals were revealed with a goat anti-rabbit Alexa 488. A positive control was performed by counterstaining for cell nuclei was performed with DAPI (eBioscience). Sections were mounted with fluorescent mounting medium (DAKO) followed by analysis using an Axioskop2 microscope with a 10× objective (Carl Zeiss AG, Jena, Germany). A stronger intensity signal in the colonic crypts region can be detected in the sections stained with CPR02, AV17 and FF06 when compared to the signal observed with an irrelevant antibody (Negative Control).

FIG. 10 shows the results of the autoradiography of the complete intestinal tract of a mouse having colitis, injected with radiolabelled anti-Agr2 antibody (CPR02) or radiolabelled anti-galectin-2 antibody (FF06). On day 12 after colitis induction, mice received 10 μg of radiolabelled IgG antibody intravenously through the lateral tail vein. Forty-eight hours later, the complete intestinal tract (comprehensive of small intestine and colon) was taken, washed with saline, and exposed to a phosphorimaging plate (Fujifilm Holdings Corp, Tokyo, Japan). All autoradiographic pictures obtained in one experiment were processed with identical parameters. A stronger intensity signal can be detected in the colon of mice injected with CPR02 or FF06 when compared to the signal in a control mouse injected with an irrelevant antibody named KSF.

### DETAILED DESCRIPTION

In one aspect, the present invention relates to antibodies which bind to galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter.

Antibody Molecule

The term “antibody molecule” describes an immunoglobulin whether natural or partly or wholly synthetically produced. The term also covers any polypeptide or protein having a binding domain which is, or is substantially homologous to, an antibody binding domain. Examples of antibody molecules are the immunoglobulin isotypes and their isotypic subclasses, as well as fragments of antibody molecules which comprise an antigen binding domain, such single chain Fvs (scFvs) and diabodies. The antibody molecule or fragment thereof may be human or humanised. The antibody molecule may be a monoclonal antibody, or a fragment thereof.

As antibodies can be modified in a number of ways, the term “antibody molecule” should be construed as covering antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

An antibody molecule as referred to herein binds galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter. These antigens have been shown to be overexpressed in the mouse intestine compared to other mouse organs and are expected to be similarly overexpressed in human intestine compared to other human organs. This differential expression makes these antigens promising targets for the delivery of therapeutic agents to the intestine. The antigen bound by the antibody molecule is thus preferably a human antigen. The names of the genes encoding these antigens in mice and humans are set out in Table 1. The sequences of human galectin-2, human IgGFc-binding protein, human galectin-4, human mucin-2, human anterior gradient protein 2 homolog, human cadherin-17, human zymogen granule membrane protein 16, human metal transporter CNNM4, human amiloride-sensitive amine oxidase [copper-containing], human hephaestin, glucosamine-fructose-6-phosphate aminotransferase [isomerizing] 1, human polymeric immunoglobulin receptor, and human sulfate transporter are known in the art and set out in SEQ ID Nos 14 to 26, respectively. The antibody molecule may thus bind to a galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter, which has, or comprises, the relevant sequence set out in SEQ ID Nos 14 to 26, or to a galectin-2, IgGFc-binding protein, galectin-4, mucin-2, anterior gradient protein 2 homolog, cadherin-17, zymogen granule membrane protein 16, metal transporter CNNM4, amiloride-sensitive amine oxidase [copper-containing], hephaestin, glucosamine-fructose-6-phosphate aminotransferase [isomerizing] 1, polymeric immunoglobulin receptor, or sulfate transporter, with an amino acid sequence which has at least 70%, more preferably one of at least 75%, 80%, 85%, 90%, 95%, 96%, 97%, 98%, 99% or 100%, sequence identity with the relevant sequence set out in SEQ ID Nos 14 to 26, respectively.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

An antibody molecule may specifically bind to its specific binding partner, e.g. galectin-2, cadherin-17, anterior gradient protein 2 homolog, or galectin-4. The term “specific” in this context may refer to the situation in which the antibody molecule will not show any significant binding to molecules other than its specific binding partner(s). The term is also applicable where e.g. an antigen-binding site of an antibody molecule is specific for a particular epitope that is carried by a number of antigens, in which case the antibody molecule carrying the antigen-binding site will be able to bind to the various antigens carrying the epitope.

Methods for isolating antibody molecules to an antigen of interest are known in the art and within the capabilities of the skilled person. For example, human hybridomas can be made as described by Kontermann & Dubel (2001) and used to isolate monoclonal antibodies to an antigen of interest. Phage display, another established technique for isolating antibody molecules to antigens of interest has been described in detail in many publications such as WO92/01047 (discussed further below) and U.S. Pat. Nos. 5,969,108, 5,565,332, 5,733,743, 5,858,657, 5,871,907, 5,872,215, 5,885,793, 5,962,255, 6,140,471, 6,172,197, 6,225,447, 6,291,650, 6,492,160, 6,521,404 and Kontermann & Dubel (2001). In addition, transgenic mice in which the mouse antibody genes are inactivated and functionally replaced with human antibody genes while leaving intact other components of the mouse immune system, can be used for isolating human antibody molecules (Mendez 1997). Antibody molecules can also be prepared synthetically, by expression from genes generated by means of oligonucleotides synthesized and assembled within suitable expression vectors, for example as described by Knappik et al. (2000) or Krebs et al. (2001).

The antibody molecule may be monovalent or bivalent i.e. may have two antigen binding sites. Where the antibody molecule is bivalent, the two antigen binding sites may be identical or different. An “antigen binding site” describes the part of an antibody which comprises the area which specifically binds to and is complementary to part or all of an antigen. Where an antigen is large, an antibody molecule may only bind to a particular part of the antigen, which part is termed an epitope. An antigen binding site may be provided by one or more antibody variable domains (e.g. a so-called Fd antibody fragment consisting of a VH domain). Preferably, an antigen binding site comprises an antibody light chain variable region (VL) and an antibody heavy chain variable region (VH).

An antibody molecule of the invention preferably comprises the HCDR3 of antibody AV17, antibody AV17.2, antibody AV17.3, antibody CPR02, antibody FF04, antibody FF05, or antibody FF06. The HCDR3 is known to play a role in determining the specificity of an antibody molecule (Segal et al., (1974), PNAS, 71:4298-4302; Amit et al., (1986), Science, 233:747-753; Chothia et al., (1987), J. Mol. Biol., 196:901-917; Chothia et al., (1989), Nature, 342:877-883; Caton et al., (1990), J. Immunol., 144: 1965-1968; Sharon et al., (1990a), PNAS, 87:4814-4817; Sharon et al., (1990b), J. Immunol., 144:4863-4869; Kabat et ai, (1991 b), J. Immunol., 147:1709-1719).

The antibody molecule may further comprise the HCDR1, HCDR2, LCDR1, LCDR2 and/or LCDR3 of antibody AV17, antibody AV17.2, antibody AV17.3, antibody CPR02, antibody FF04, antibody FF05, or antibody FF06.

The antibody may also comprise the VH and/or VL domain, or scFv sequence, of antibody AV17, antibody AV17.2, antibody AV17.3, antibody CPR02, antibody FF04, antibody FF05, or antibody FF06.

An antibody molecule of the invention may comprise a VH domain having at least 70%, more preferably one of at least 75%, 80%, 85%, 90%, 95%, 96%, 97%, 98%, 99% or 100%, sequence identity to the VH domain of antibody AV17, antibody AV17.2, antibody AV17.3, antibody CPR02, antibody FF04, antibody FF05, or antibody FF06.

An antibody molecule of the invention may comprise a VL domain having at least 70%, more preferably one of at least 75%, 80%, 85%, 90%, 95%, 96%, 97%, 98%, 99% or 100%, sequence identity to the VL domain of antibody AV17, antibody AV17.2, antibody AV17.3, antibody CPR02, antibody FF04, antibody FF05, or antibody FF06.

An antibody molecule of the invention may be or comprise an scFv having at least 70%, more preferably one of at least 75%, 80%, 85%, 90%, 95%, 96%, 97%, 98%, 99% or 100%, sequence identity to the scFv sequence of antibody AV17, antibody AV17.2, antibody AV17.3, antibody CPR02, antibody FF04, antibody FF05, or antibody FF06.

Variants of these VH and VL domains and CDRs may also be employed in antibody molecules for use in as described herein. Suitable variants can be obtained by means of methods of sequence alteration, or mutation, and screening.

Particular variants for use as described herein may include one or more amino acid sequence alterations (addition, deletion, substitution and/or insertion of an amino acid residue), maybe less than about 20 alterations, less than about 15 alterations, less than about 10 alterations or less than about 5 alterations, 4, 3, 2 or 1.

Alterations may be made in one or more framework regions and/or one or more CDRs. In particular, alterations may be made in HCDR1, HCDR2 and/or HCDR3.

The antibody molecule may be a whole antibody or a fragment thereof, in particular an antigen-binding fragment thereof. Whole antibody molecules include IgA, IgD, IgE, IgG or IgM. Preferably, the whole antibody molecule is IgG.

The antibody molecule may be an antigen-binding fragment of a whole antibody molecule. Antigen-binding fragments of whole antibodies include (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al. (1989) Nature 341, 544-546; McCafferty et al., (1990) Nature, 348, 552-554; Holt et al. (2003) Trends in Biotechnology 21, 484-490), which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science, 242, 423-426; Huston et al. (1988) PNAS USA, 85, 5879-5883); (viii) bispecific single chain Fv dimers (PCT/US92/09965) and (ix) “diabodies”, multivalent or multispecific fragments constructed by gene fusion (WO2013/014149; WO94/13804; Holliger et al. (1993a), Proc. Natl. Acad. Sci. USA 90 6444-6448). Fv, scFv or diabody molecules may be stabilized by the incorporation of disulphide bridges linking the VH and VL domains (Reiter et al. (1996), Nature Biotech, 14, 1239-1245). Minibodies comprising a scFv joined to a CH3 domain may also be made (Hu et al. (1996), Cancer Res., 56(13):3055-61). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

A single chain Fv (scFv) may be comprised within a mini-immunoglobulin or small immunoprotein (SIP), e.g. as described in (Li et al., (1997), Protein Engineering, 10: 731-736). An SIP may comprise an scFv molecule fused to the CH4 domain of the human IgE secretory isoform IgE-S2 (εₛ₂-CH4; Batista et al., (1996), J. Exp. Med., 184: 2197-205) forming an homo-dimeric mini-immunoglobulin antibody molecule

Preferably, the antibody molecule is, or comprises, a single chain Fv (scFv), is a small immunoprotein (SIP), is a diabody, or is an IgG molecule.

Where the antibody molecule is a diabody, the VH and VL domains are preferably linked by a 5 to 12 amino acid linker. A diabody comprises two VH-VL molecules which associate to form a dimer. For example, the VH and VL domains may be linked by an amino acid linker which is 5, 6, 7, 8, 9, 10, 11, or 12 amino acid in length. Preferably, the amino acid linker is 5 amino acids in length. Suitable linker sequences are known in the art.

Where the antibody molecule is an scFv, the VH and VL domains of the antibody are preferably linked by a 14 to 20 amino acid linker. For example, the VH and VL domains may be linked by an amino acid linker which is 14, 15, 16, 17, 18, 19, or 20 amino acid in length. Suitable linker sequences are known in the art. For example, the linker may have the sequence set forth in SEQ ID NO: 40.

Conjugates

In the context of the present invention, an antibody molecule may be conjugated to an anti-inflammatory or immunosuppressive molecule. Preferably, the antibody molecule is conjugated to a cytokine, most preferably an anti-inflammatory cytokine. The cytokine is preferably a human cytokine.

Cytokines with anti-inflammatory properties include interleukin-4 (IL-4), interleukin-10 (IL-10), interleukin-12 (IL-12), interleukin-22 (IL-22), interleukin-27 (IL-27), interleukin-35 (IL-35), granulocyte-macrophage colony-stimulating factor (GM-CSF), and interleukin-33 (IL-33). The sequences of these cytokines are well known in the art.

The antibody molecule and the anti-inflammatory or immunosuppressive molecule may be connected to each other directly, for example through any suitable chemical bond, e.g. a peptide bond, or through a linker, such as an amino acid linker.

The amino acid linker may be a short (2-20, preferably 2-15, amino acids long). Suitable examples of amino acid linker sequences are known in the art. One or more different linkers may be used.

The chemical bond may be, for example, a covalent or ionic bond. Examples of covalent bonds include peptide bonds (amide bonds) and disulphide bonds. For example the antibody molecule and therapeutic or diagnostic agent may be covalently linked. For example by peptide bonds (amide bonds).

Where the antibody molecule is conjugated to the anti-inflammatory or immunosuppressive molecule by means of a peptide bond or amino acid linker, the antibody molecule may form part of a fusion protein comprising the antibody molecule and the anti-inflammatory or immunosuppressive molecule. In this case, the antibody molecule and anti-inflammatory or immunosuppressive molecule may be produced (secreted) as a single chain polypeptide.

The immunosuppressive or anti-inflammatory agent may be conjugated, either through an amino acid linker, or directly, to the N-terminus or C-terminus of the antibody molecule. For example, where the antibody molecule is or comprises an scFv, the immunosuppressive or anti-inflammatory agent may be conjugated to the N-terminus of the VH domain of the scFv, or to the C-terminus of the VL domain of the scFv. Where the antibody molecule is a diabody (which comprises two scFv molecules), the immunosuppressive or anti-inflammatory agent may be conjugated to the N-terminus of one or both of the VH domains, or to the C-terminus of one or both of the VL domains, of the two scFvs making up the diabody.

Other means for conjugation to antibody molecules are known in the art and include chemical conjugation, especially cross-linking using a bifunctional reagent, e.g. employing DOUBLE-REAGENTS™ Cross-linking Reagents Selection Guide, Pierce.

Methods of Treatment

An antibody molecule or conjugate as described herein may be used in a method of treatment (which may include prophylactic treatment) of an intestinal disease or disorder in a patient (typically a human patient). The method comprises administering a therapeutically effective amount of the antibody molecule or conjugate to the patient.

The intestinal disease or disorder may an inflammatory disease or disorder, and/or an autoimmune disease. Examples include inflammatory bowel disease (IBD) and irritable bowel syndrome (IBS). IBD is thought to be both an inflammatory and an autoimmune disease, and examples include Crohn's disease and ulcerative colitis (UC). IBS is an inflammatory disorder.

The major types of IBD are Crohn's disease and ulcerative colitis, while other types of IBD include collagenous colitis, lymphocytic colitis, ischaemic colitis, diversion colitis, Behçet's disease and indeterminate colitis. Crohn's disease can affect any part of the gastrointestinal tract, whereas ulcerative colitis is typically restricted to the colon and rectum.

IBD, as referred to herein, may be Crohn's disease, ulcerative colitis, collagenous colitis, lymphocytic colitis, ischaemic colitis, diversion colitis, Behçet's disease or indeterminate colitis. In particular, the terms Crohn's disease, ulcerative colitis, collagenous colitis, lymphocytic colitis, ischaemic colitis, diversion colitis, Behçet's disease and indeterminate colitis, as used herein, may refer to active Crohn's disease, active ulcerative colitis, active collagenous colitis, active lymphocytic colitis, active ischaemic colitis, active diversion colitis, and active indeterminate colitis, respectively.

The intestinal disease or disorder is preferably inflammatory bowel disease, such as ulcerative colitis or Crohn's disease.

The antibody molecule or conjugate may be in form of a pharmaceutical composition. The pharmaceutical composition typically comprises a therapeutically effective amount of the antibody molecule or conjugate and optionally auxiliary substances such as pharmaceutically acceptable excipient(s). Pharmaceutical compositions are prepared in a manner well known in the pharmaceutical art. A carrier or excipient may be a liquid material which can serve as a vehicle or medium for the active ingredient. Suitable carriers or excipients are well known in the art and include, for example, stabilisers, antioxidants, pH-regulating substances, controlled-release excipients. The pharmaceutical composition may be adapted, for example, for parenteral use and may be administered to the patient in the form of solutions or the like.

Pharmaceutical compositions comprising an antibody molecule or conjugate as described herein invention may be administered to a patient. Administration is preferably in a “therapeutically effective amount”, this being sufficient to show benefit to the patient. Such benefit may be amelioration of at least one symptom. The actual amount administered, and rate and time-course of administration, will depend on the nature and severity of what is being treated. Prescription of treatment, e.g. decisions on dosage etc., is within the responsibility of general practitioners and other medical doctors. Treatments may be repeated at daily, twice-weekly, weekly, or monthly intervals at the discretion of the physician.

A pharmaceutical composition may be administered to a patient in need of treatment via any suitable route, usually by injection into the bloodstream and/or directly into the site to be treated. The precise dose and its frequency of administration will depend upon a number of factors, the route of treatment, the size and location of the area to be treated.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included

For intravenous injection, or injection at the site of affliction, the pharmaceutical composition will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

A pharmaceutical composition may be administered alone or in combination with other treatments, either simultaneously or sequentially dependent upon the condition to be treated.

Kits

An antibody molecule or conjugate as described herein may in the form of a therapeutic kit for use in the treatment of an intestinal disease or disorder in a patient. The components of a kit are preferably sterile and in sealed vials or other containers.

A kit may further comprise instructions for use of the components in a method described herein. The components of the kit may be comprised or packaged in a container, for example a bag, box, jar, tin or blister pack.

Further aspects and embodiments of the invention will be apparent to those skilled in the art given the present disclosure including the following experimental exemplification.

All documents mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### EXAMPLES

### Example 1: Identification of Proteins Overexpressed in Human Intestine

Materials and Methods

In Vivo Perfusion Procedure

Mice were anesthetized with 220-300 mg/kg ketamine, 15 mg/kg xylazine and 3 mg/kg acepromazine. The chest of the anesthetized mouse was opened through a median sternotomy. The left heart ventricle was punctured with a perfusion needle (25-gauge butterfly cannula fitted to a barb) and a small cut was made in the right atrium to allow outflow of the perfusion solutions. All perfusions were performed at 100 mm Hg. Blood components were washed away with prewarmed PBS (38° C.) supplemented with 10% (wt/vol) dextran 40 as plasma expander for 10 min. Immediately afterwards, the mouse was perfused with 7 ml prewarmed (38° C.) biotinylation solution (1 mg/ml Sulpho-NHS-LC-Biotin in PBS (pH 7.4), 10% (wt/vol) dextran 40) at a flow rate of approx. 1.5 ml/min. To neutralize unreacted biotinylation reagent, the mice were then perfused with 10 ml prewarmed (38° C.) 50 mM Tris in PBS, 10% (wt/vol) dextran 40. After the neutralization of unreacted biotinylation reagent, organs and tumors were excised and specimens were snap-frozen for preparation of organ homogenates.

Homogenization of Organs

Organs were cut in ≤1 mm³ pieces on ice and 100 mg of cut tissue was resuspended in lysis buffer (2% sodium dodecyl sulfate, 50 mM Tris, 10 mM ethylenediaminetetraacetic acid, Roche Complete EDTA free proteinase inhibitor cocktail in PBS, pH 7.5), using 20 μL buffer per mg tissue. Tissue was homogenized using a Tissue Lyser II (Qiagen) and sonicated using a Vibra-cell sonicator (Sonics), followed by 20 minutes of incubation at 95° C. and 20 minutes of centrifugation at 16 300 g. The resulting supernatants (total protein extracts) were stored at −80° C. The protein concentration of the total protein extracts was determined with the BCA Protein Assay Reagent Kit (Pierce).

Purification of Biotinylated Proteins and Tryptic Digest

For each sample, 400 μL streptavidin-sepharose (GE Healthcare) slurry were washed thrice in buffer A (1% NP40, 0.1% SDS in PBS), pelleted, and mixed with 5 mg of total protein extract. The capture of biotinylated proteins was allowed to proceed for 2 h at room temperature in a revolving mixer. The supernatant was removed, and the resin was washed thrice with buffer A, twice with High pH buffer (50 mM Ammonium Bicarbonate, pH 10), twice with High salt buffer (2 M NaCl in PBS), and eight times with digestion buffer (50 mM Tris-HCl, 1 mM CaCl₂); pH 8.0) using an UniVac3 vacuum manifold (Whatman Int Ltd). Finally, the resin was resuspended in 200 μL of digestion buffer and 20 μL of trypsin stock solution (80 ng/μL sequencing grade-modified porcine trypsin (Promega) in digestion buffer) were added. Protease digestion was carried out overnight at 37° C. under constant agitation. Peptides were desalted, purified, and concentrated with C18 microcolumns (OMIX tips; Varian, Inc.). After lyophilization, peptides were stored at −20° C. Prior to HPLC analysis, lyophilized peptides were dissolved in 23 μL of 0.1% TFA in Chromasolv water.

Nanocapillary HPLC with Automated On-Line Fraction Spotting onto MALDI Target Plate Tryptic peptides were separated by reverse phase nano-HPLC using an EASY-nLC system (Proxeon). Mobile phase A consisted of 0.1% TFA in water, mobile phase B of 0.1% TFA in ACN. The flow rate was 300 nL/min. Lyophilized peptides derived from the digestion of biotinylated protein affinity purified from 5 mg of total protein extracts were dissolved in 23 μL of buffer A and 18 μL of this solution were loaded on the column (ReproSil-Pur 120 C18-AQ, 3 μm; Dr. Maisch GmbH). Peptides were eluted with the following elution gradient: 5-33% B for 62 min, 33-48% B for 15 min, 48-100% B for 2 min and 100% B for 10 min. Before analyzing the next sample, the column was equilibrated with 5% B for 20 min. Eluting fractions were mixed with a solution of 3 mg/mL CHCA, 187.5 pmol/mL of each of the four internal standard peptides ([des-Arg9]-bradykinin, neurotensin, angiotensin I, and adrenocorticotropic hormone fragment 1-17; all from Sigma), 0.1% TFA, and 70% ACN in water and deposed on a blank MALDI target plate (416 spots per sample) using an online SunCollect system (SunChrom). The final concentration of each internal standard peptide was 50 fmol per spot.

Mass Spectrometric Analysis and Peptide Annotation

MALDI-TOF/TOF analysis was carried out with the 4800 MALDI TOF/TOF Analyzer (Applied Biosystems). All spectra were acquired with a solid-state laser (355 nm) at a laser repetition rate of 200 Hz. After measuring all samples in the MS mode, a maximum of 12 precursors per spot were automatically selected for subsequent fragmentation by CID by the mass spectrometer's control software (4000 Series Explorer V3.5, Applied Biosystems). The resulting spectra were processed and analyzed using the Global Protein Server Workstation version 3.6 (GPS Explorer, Applied Biosystems), which uses internal MASCOT version 2.1 software (Matrix Sciences, London, UK) for matching MS and MS/MS data against databases of in silico digested proteins. Only fully tryptic peptides were taken into account. The MS/MS data were searched against databases of mouse proteins downloaded from the Uni-Prot homepage. Furthermore, following analysis settings were used for the identification of peptides and proteins: (i) precursor tolerance: below 12 ppm, (ii) MS/MS fragment tolerance: 0.5 Da, (iii) maximal missed cleavages: 1 and (iv) one variable modification (oxidation of methionine). The four spike proteins were added to the above-described databases for the annotation of MS/MS spectra to peptides and proteins. Peptides were considered correct calls when the confidence interval was greater than 95%.

Relative Protein Quantification by DeepQuanTR Software

After MS acquisition, Peak detection, S/N calculations and deisotoping were performed by the Data Explorer Software (Applied Biosystems). Peak information (fractions, intensities, m/z ratios) was loaded into the DeepQuanTR software suite in .txt format following the export from the Data Explorer software. To cope with ion suppression effects and spot-to-spot intensity differences, standard peptides spiked in constant amounts to each eluting HPLC fraction were used for signal intensity normalization. The peak intensities of each spectrum were normalized to the four internal standard peptides [des-Arg⁹]-bradykinin, angiotensin I, neurotensin and adrenocorticotropic hormone fragments 1-17.

For each sample, the feature extraction algorithm grouped MS signals with similar m/z ratios present in spectra of subsequent HPLC fractions into features if within a m/z tolerance range of 12 ppm. The samples were aligned and grouped based on their tissue origin. To annotate peptide and protein information to features, a list of identified peptides (containing m/z ratios and HPLC fraction information) was matched to a list of features within a tolerance of one fraction and 20 ppm mass difference. For peptides which could not be assigned to a feature, tolerances were consecutively increased to a maximum of five fractions and 40 ppm mass difference.

Following annotation of peptide and protein information, the relative quantification was carried out. Importantly, an improved algorithm for the calculation of protein regulations (i.e., protein quant values) was applied. Instead of calculating protein quant values based on the individual average peptide ratios of all peptides annotated to a given protein, peptide quant values (i.e., the sum of the normalized signal intensities of all peaks belonging to a given peptide) for all peptides annotated to a protein were first summed up (empty values were set to 1, that is the background value) and the ratio between these values was calculated. This alternative approach was identified to be less sensitive to outliers and to perform superior in label-free quantification experiments.

Peptides were newly assembled to proteins using the corresponding feature integrated in DeepQuanTR. Briefly, a minimal protein list was established by annotating peptides to proteins and clustering of proteins sharing one or more peptides. If a protein was identified by at least one unique peptide, it was added to the protein list presented by DeepQuanTR.

Results

Organs collected from mice (adrenal gland, aorta, brain, heart, intestine [including colon and duodenum], kidney, liver, lung, muscle, ovary, and spleen), following in vivo perfusion with reactive biotin derivatives were lysed and biotinylated proteins captured on streptavidin resin. After stringent washing, proteins were digested with trypsin. Tryptic peptides were purified and analyzed by liquid chromatography and mass spectrometry. Mass spectrometric data was processed with the DeepQuanTR software suite allowing for the relative quantification of proteins identified in all samples. A total of 2263 proteins expressed in one or more of the organs were identified, quantified, and used to prepare an organ atlas containing protein expression data for the above mentioned organs. The materials and methods were as set out in the materials and methods section.

The organ atlas was then used to identify thirteen proteins which demonstrate a putative extracellular or plasma membrane localization and significant expression in intestine as compared to other organs. All thirteen of these proteins were at least 7.5-fold more abundant in intestine as compared to any other organ in the organ atlas (see Table 1).

The sequences of the mouse proteins listed in Table 1 are set out in SEQ ID NOs 1 to 13. Corresponding human proteins also exists and their sequences are listed in SEQ ID NOs 14 to 26. Like in the mouse, these proteins are expected to be overexpressed in human intestine relative to other human organs. This makes these proteins promising targets for antibody-mediated pharmacodelivery of therapeutic agents, such as anti-inflammatory molecules, to the human intestine to treat intestinal diseases and disorders.

TABLE 1
Proteins identified from the organ atlas with the strongest expression in intestine as
compared to other organs. Regulation is expressed as the natural logarithm of the relative
protein abundance in the respective organ.

| | | | | | | | | | | | | | Regulation |
|---|---|---|---|---|---|---|---|---|---|---|---|---|---|
| | Gene | Adrenal | | | | | | | | | | | (intestine vs |
| Protein Name | Name | gland | Aorta | Brain | Heart | Intestine | Kidney | Liver | Lung | Muscle | Ovary | Spleen | other organs) |
| Galectin-2 | LGALS2 | 1.8 | −2.9 | 1.9 | −2.9 | 7.0 | 0.0 | 1.8 | 1.9 | −2.9 | −2.9 | −2.9 | 155.6 |
| IgGFc-binding protein | FCGBP | −3.0 | −3.0 | −3.0 | −3.0 | 8.2 | −3.0 | 2.2 | 1.7 | 2.4 | 3.3 | −3.0 | 130.7 |
| Galectin-4 | LGALS4 | 0.0 | −4.4 | 1.7 | 0.8 | 6.4 | −4.4 | 1.0 | −0.3 | −0.2 | 0.1 | −0.8 | 114.8 |
| Mucin-2 | MUC2 | −1.8 | −1.8 | 0.6 | 1.3 | 7.5 | −1.8 | −1.8 | −1.8 | 2.8 | −1.8 | −1.8 | 112.5 |
| Anterior gradient | AGR2 | −1.4 | −1.4 | −1.4 | −1.4 | 8.6 | −1.4 | −1.4 | 4.4 | −1.4 | −1.4 | −1.4 | 64.5 |
| protein 2 homolog | | | | | | | | | | | | | |
| Cadherin-17 | CDH17 | −0.1 | 0.8 | −1.8 | −0.2 | 4.3 | −0.9 | 0.3 | −1.5 | −1.4 | 0.2 | 0.3 | 33.6 |
| Zymogen granule | ZG16 | −1.3 | −1.3 | −1.3 | −1.3 | 7.3 | −1.3 | −1.3 | 4.0 | −1.3 | −1.3 | −1.3 | 27.1 |
| membrane protein 16 | | | | | | | | | | | | | |
| Metal transporter | CNNM4 | −3.2 | −3.2 | 2.7 | −3.2 | 7.6 | 4.4 | −3.2 | −3.2 | −3.2 | 3.6 | 0.8 | 23.2 |
| CNNM4 | | | | | | | | | | | | | |
| Amiloride-sensitive | ABP1 | −1.4 | −0.9 | −1.6 | −1.2 | 5.3 | −0.3 | −0.7 | 2.6 | −0.9 | 0.5 | −1.4 | 14.9 |
| amine oxidase [copper- | | | | | | | | | | | | | |
| containing] | | | | | | | | | | | | | |
| Hephaestin | HEPH | 0.6 | −1.0 | −5.7 | 0.2 | 5.8 | 1.3 | −1.6 | 1.2 | −5.7 | 3.1 | 1.7 | 14.2 |
| Glucosamine-fructose- | GFPT1 | −1.8 | −1.8 | −1.8 | −1.8 | 5.7 | 1.5 | 3.5 | −1.8 | 2.0 | −1.8 | −1.8 | 9.0 |
| 6-phosphate | | | | | | | | | | | | | |
| aminotransferase | | | | | | | | | | | | | |
| [isomerizing] 1 | | | | | | | | | | | | | |
| Polymeric | PIGR | 1.4 | 0.1 | −0.8 | −0.3 | 4.5 | −1.0 | 2.0 | −4.8 | −4.8 | 1.2 | 2.5 | 7.8 |
| immunoglobulin | | | | | | | | | | | | | |
| receptor | | | | | | | | | | | | | |
| Sulfate transporter | SLC26A2 | 1.4 | −0.7 | 1.0 | −1.9 | 4.9 | −5.8 | −1.2 | 2.9 | −0.8 | −0.3 | 0.5 | 7.6 |

### Example 2: Preparation and Characterization of Three New Antibodies Against Cadherin-17

Materials and Methods

Antigen Preparation for Antibody Phage Display Selection

For the generation of fully human monoclonal antibodies against human cadherin-17. A recombinant version of domain 1-2 of cadherin-17 (located in the extra-cellular part of the protein) was produced in CHO-S cells by transient gene expression, and in E. coli BL21 cells. The antigen was purified by means of a 6×histidine-tag appended to the C-terminus of the protein. For antibody selection, the antigen was immobilized on plastic (MaxiSorp, NUNC) at a concentration of 10⁶ M. The amino acid sequence of domain 1-2 of human cadherin-17 is set out in SEQ ID NO: 27.

Antibody Selection

ScFv selection was performed as described in Silacci et al, (Proteomics 2005, 5, 2340-2350) using an scFv antibody library as disclosed in WO2010/028791. The antibody selection was essentially as follows. Immunomodules (Nunc; Thermo Scientific, Denmark) were coated with domain 1-2 of human cadherin-17 at a concentration of 50 mg/ml in PBS, at room temperature overnight. Immunomodules were then rinsed with PBS and blocked for 2 h at room temperature with 2% w/v skimmed milk in PBS (MPBS). After rinsing with PBS, 10¹² phage particles in 2% MPBS were added to the immunomodules. The immunomodules were first incubated at room temperature on a shaker for 30 min and then for 1.5 h standing still. Unbound phage was washed away by rinsing the immunomodules ten times with PBS 0.1% Tween-20 and ten times with PBS. The bound phage was eluted in 1 ml of 100 mM triethylamine by inverting the tube for 5 min. Triethylamine was neutralized by adding 0.5 mL 1 M Tris-HCl (pH 7.4). The eluted phage was used for the infection of exponentially growing E. coli TG1.

ELISA

Bacterial supernatants containing scFv fragments were screened for binding to antigen by ELISA as described in Silacci et al. (2005). Individual colonies were inoculated in 180 μl 2TY medium comprising 100 μg/ml ampicillin (Applichem; Darmstadt, Germany) and 0.1% glucose (Sigma) in 96-well plates (Nunclon Surface, Nunc). The plates were incubated for 3 h at 37° C. in a shaker incubator. The cells were then induced with isopropyl-thio-galactopyranoside (IPTG; Applichem) at a final concentration of 1 mM, and grown overnight at 30° C. The bacterial supernatants were tested in ELISAs as described in Silacci et al. (2005), using the anti-myc tag 9E10 mAb (1 μg/ml) and anti-mouse horseradish peroxidase immunoglobulins (A2554 Sigma) as secondary reagents. The read out was OD450 nm (minus background signal at 620 nm).

Expression and Purification of scFv Antibody Fragments

ScFv antibody fragments from selected bacterial clones were produced by inoculating a single fresh colony in 10 mL 2TY medium, comprising 100 μg/ml ampicillin and 1% glucose. This pre-culture was grown overnight at 37° C. then diluted 1:100 in 800 ml 2TY medium, 100 μg/ml ampicillin, 0.1% glucose and grown at 37° C. until is reached the exponential phase. The cells were then induced by the addition of IPTG (final concentration 1 mM) and grown at 30° C. overnight. The scFv fragments were purified from the bacterial supernatant by affinity chromatography using Protein A Sepharose (Sino Biological Inc.) according to the manufacturer's instructions.

Size-Exclusion Chromatography and BiaCore Analysis

For the analysis of the AV17 antibody, size-exclusion chromatography was performed on an ÄKTA FPLC system using a Superdex 75 column (Amersham Biosciences). Surface plasmon resonance affinity measurements were performed using a Biacore S200 instrument and purified scFvs. Monomeric scFv were injected as a serial-dilution, with concentrations ranging from 950 nM to 9.5 nM.

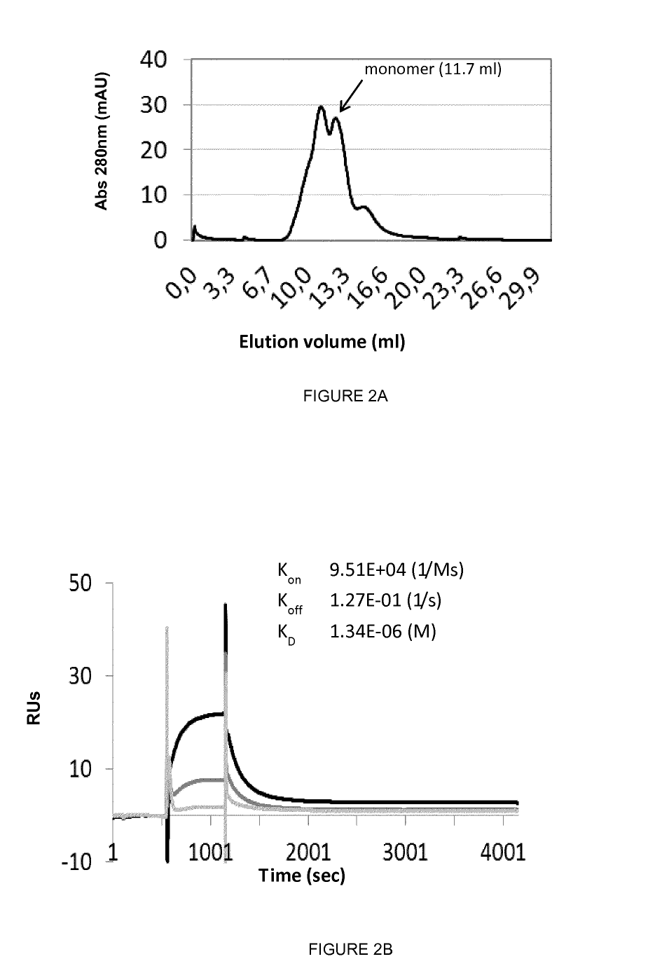

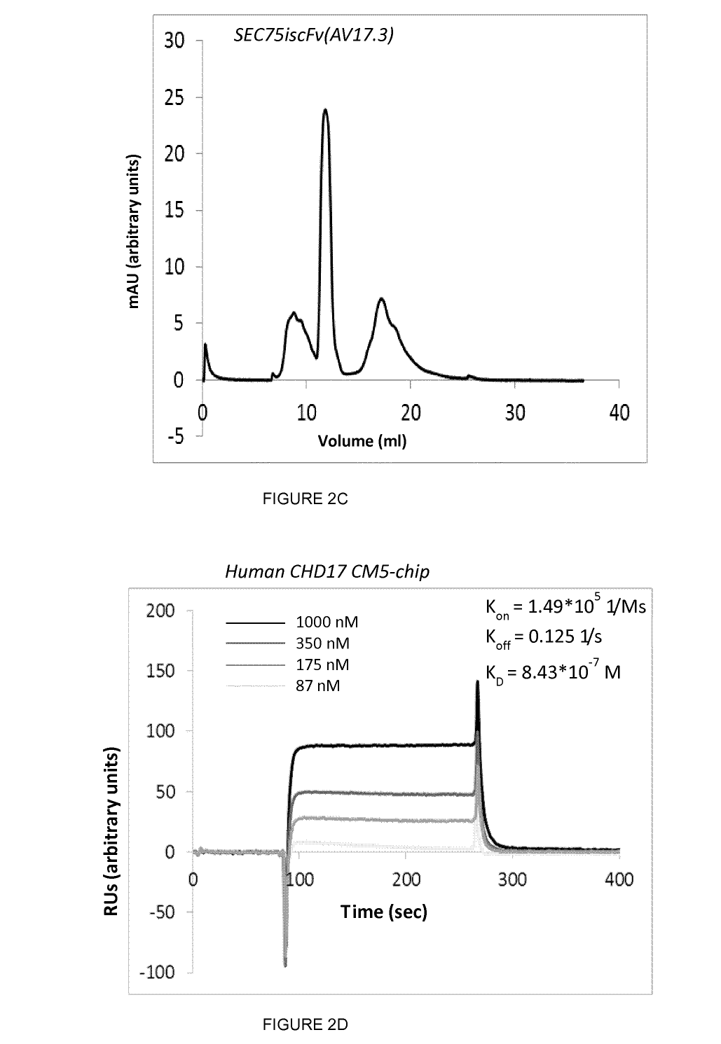

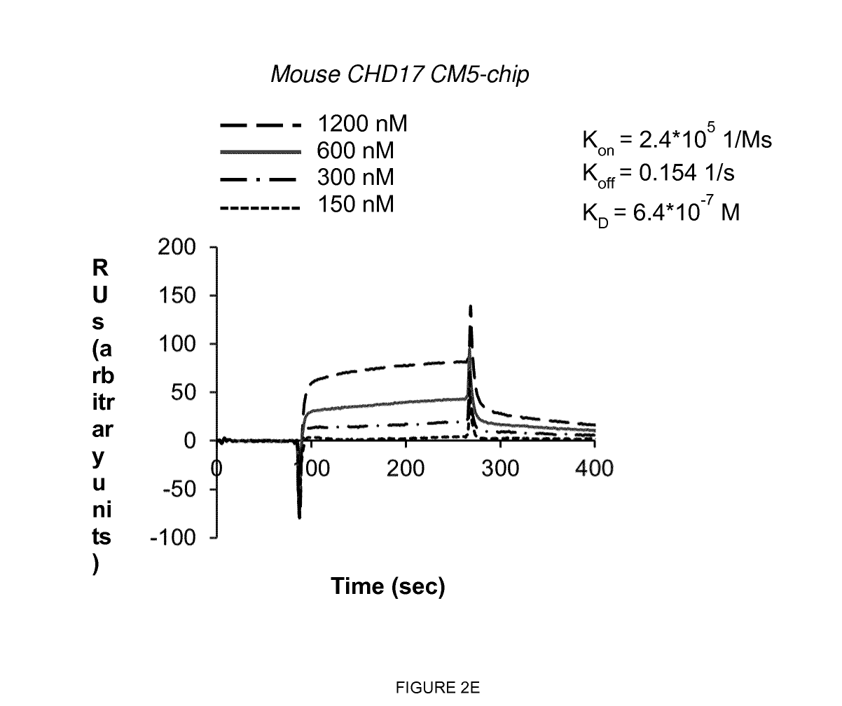

For the analysis of the AV17.3 antibody, size-exclusion chromatography was performed on an ÄKTA FPLC system using the Superdex 75 Increase column (Amersham Biosciences). Surface plasmon resonance experiments affinity measurements were performed by BIAcore X100 instrument with purified scFvs. Human Cadherin-17 or mouse Cadherin-17 was immobilized on a CM5 sensor chip (GE Healthcare) and monomeric scFv (AV17.3) were injected as serial-dilution. The concentrations are reported on FIG. 2D and FIG. 2E.

Immunofluorescent Staining

Freshly frozen human biopsy samples of Ulcerative Colitis were stained as described in the following lines. In brief, purified antibodies in human IgG1 format were added at the final concentration of 2 μg/ml to the sections. Detection of the primary antibody was performed rabbit anti human-IgG (DAKO) and signals were revealed with a goat anti-rabbit Alexa 488. Positive control was performed by staining blood vessels with an antibody (eBioscience, 1:100) specific for CD31, an endothelial cell marker, the signal revealed with goat anti-mouse Alexa-594 (Invitrogen). Further positive control was performed by counterstaining for cell nuclei was performed with DAPI (eBioscience). Sections were mounted with fluorescent mounting medium (DAKO) followed by analysis using an Axioskop2 microscope with a 10× objective (Carl Zeiss AG, Jena, Germany).

Results

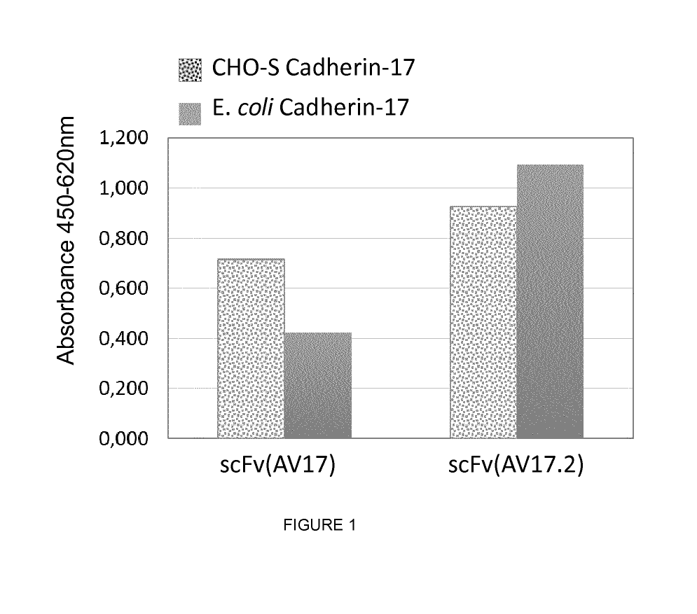

Three scFvs, scFv(AV17), scFv(AV17.2) and scFv(AV17.3) were isolated from the antibody library following two rounds of panning using domain 1-2 of human cadherin-17 (CHO-S expressed). ScFv(AV17) and scFv(AV17.2) were capable of binding human cadherin-17 domain 1-2 produced by CHO-S cells (glycosilated protein, 2 N-linked glycosilation sites) or E. coli BL21 as determined by ELISA. The results are shown in FIG. 1.

In ELISAs, both scFv(AV17) and scFv(AV17.2) displayed a binding affinity for human cadherin-17 between 1.5-0.5×10⁻⁶M (data not shown). A more precise affinity constant determination was performed by BiaCore analysis with the monomeric scFv(AV17) obtained by size exclusion chromatography (SEC-75, Amersham Biosciences) on a human cadherin-17 coated chip. The results are shown in FIGS. 2A and 2B, and demonstrate that monomeric scFv(AV17) had an affinity (K_{D}) for human cadherin-17 of 1.34×10⁻⁶. For the AV17.3 antibody, the affinity constant determination was performed by BiaCore analysis with the monomeric scFv(AV17.3) obtained by size exclusion chromatography (SEC-75i, Amersham Biosciences) on a human cadherin-17 coated chip and a mouse cadherin-17 coated chip. The results are shown in FIGS. 2C, 2D and 2E, and the Biacore analysis demonstrates that scFv(AV17.3) had an affinity (K_{D}) for the human Cadherin-17 of 8.43×10⁻⁷ M and a K_{D} for the murine Cadherin-17 of 6.4×10⁻⁷ M and thus the species cross-reactivity of this antibody.

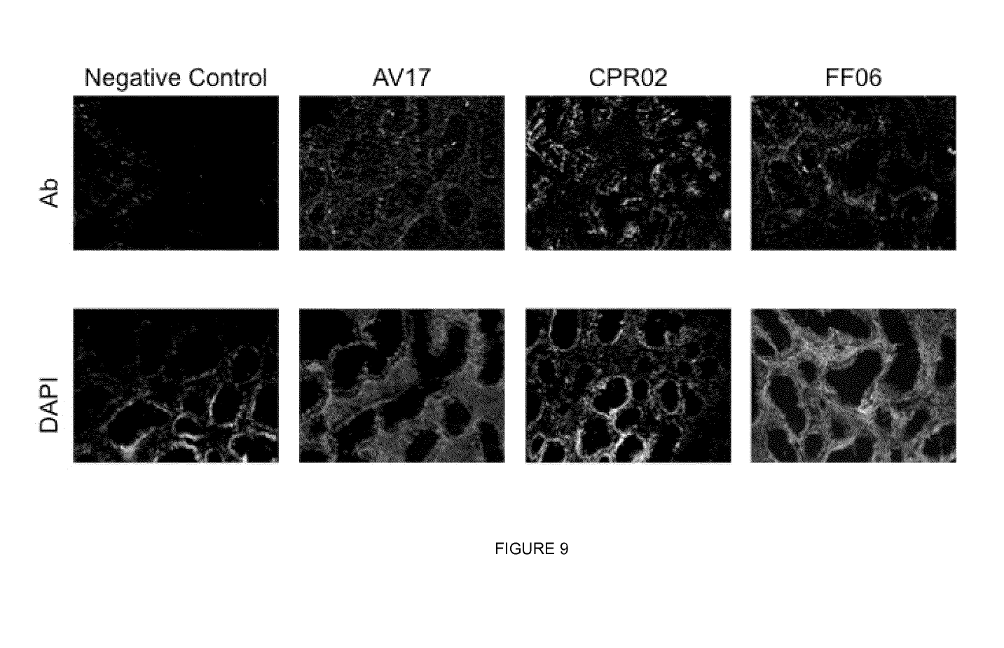

In order to verify the ability of scFv(AV17) to recognize their cognate antigen in a more physiological environment, immunofluorescence analysis was carried out on human biopsy samples of Ulcerative Colitis. As can be seen in FIG. 9, a stronger intensity signal in the colonic crypts region can be detected in the sections stained with AV17 when compared to the signal observed with an irrelevant antibody (Negative Control).

### Example 3: Preparation and Characterization of a New Antibody Against Human Anterior Gradient Protein 2 Homolog (Agr2)

Materials & Methods

Antigen Preparation for Antibody Phage Display Selections

For the generation of fully human monoclonal antibodies against human Agr2, a recombinant version of an N-terminally truncated form of the protein was produced in a bacterial expression system. Amino acid residues 41-175 of human Agr2 were expressed in a BL21-based E. coli expression strain and purified using a 6×Histidine-tag appended to the C-terminus of the protein.

The antigen was biotinylated by modification of surface lysine residues with an NHS-LC-biotin reagent (Thermo Fisher). The biotinylated antigen was used at a concentration of 0.1 μM for selections. The amino acid sequence of residues 41-175 of recombinant Agr2 is set out in SEQ ID NO: 66.

Antibody Selection

Biotinylated Agr2 at a concentration of 0.1 μM was immobilized onto streptavidin-coated M-280 dynabeads (Thermo Fisher). The coated beads were blocked with 2% w/v skimmed milk powder in phosphate-buffered saline (MPBS). The blocked beads were incubated with 10¹² phage particles from naïve phage libraries as disclosed in WO2010/028791 diluted in MPBS for 1.5 hr. Unbound phage were removed by washing with PBS+0.1% Tween-20 followed by washing with PBS alone. Bound phages were eluted by incubation of the beads with 800 μL 100 mM trimethylamine for 5 min. The eluted phage solution was neutralized by addition of 0.4 mL 1 M Tris-HCl pH 7.4. Eluted phages were recovered through infection of exponentially growing E. coli TG1 cells.

Expression and Purification of scFv Antibody Fragments

ScFv antibody fragments from selected bacterial clones were produced by inoculating 10 mL of 2TY medium containing 100 μg/ml ampicillin and 1% v/v glucose with a single bacterial colony containing the scFv expression plasmid. The pre-culture was incubated at 37° C., 180 rpm for 4 hr until turbid, and then diluted 1:100 into 800 mL 2TY medium comprising 100 μg/ml ampicillin and 1% v/v glucose. The large scale culture was incubated at 37° C., 180 rpm until the culture reached mid-exponential phase, and protein expression was then induced with IPTG at a final concentration of 1 mM. The induced culture was incubated overnight at 30° C., 180 rpm. The scFv fragments were purified from the bacterial supernatant by affinity chromatography using Protein A Sepharose (Sino Biological Inc.) according to the manufacturer's instructions.

ELISA

Binding of the purified scFvs to the recombinant Agr2 antigen (consisting of amino acid residues 41-175 of Agr2) was confirmed by ELISA. Maxisorp plates (Nunc) were incubated with avidin at 50 μg/mL in PBS for 6 hours at room temperature. Unbound avidin was removed by washing with PBS and the wells were incubated with biotinylated Agr2 at a final concentration of 0.2 μM overnight at 4° C. Unbound Agr2 was removed by washing with PBS, and coated wells were blocked for 1 hr with 2% w/v skimmed milk powder in phosphate-buffered saline (MPBS). Serial dilutions of the purified scFv antibodies were prepared in MPBS at concentrations between 1 μM and 0.02 μM. The anti-myc tag antibody 9E10 was added to the prepared antibody dilutions at a final concentration of 1 μg/ml and the mixture was incubated with the immobilized Agr2 antigen for 1.5 hr at room temperature. Bound scFvs were detected using a 1:1000 dilution of HRP-conjugated anti-murine Fc (Sigma Aldrich) and the signal was developed using BM Blue POD substrate (Roche). The ELISA output was recorded at a wavelength of 450 nM, subtracting absorbance at 620 nM as a reference baseline.

Surface Plasmon Resonance

Binding of the purified ScFvs to the recombinant human Agr2 antigen was confirmed by surface plasmon resonance experiments using a BIAcore T3000 instrument (GE Healthcare). Human Agr2 was immobilized on a CM5 sensor chip (GE Healthcare), and serial dilutions of the purified ScFv antibodies at concentrations between 3400 nM and 213 nM were injected over the surface.

Immunofluorescence Staining

(i) Freshly frozen colon biopsy samples from a healthy donor were stained according to already published methods (Pfaffen et al., Eur J Nucl Med Mol Imagin (2010) 37: 1559-65). Briefly, purified biotinylated antibodies in IgG format were added at the final concentration of 2 μg/ml onto the sections. Detection of the primary antibody was performed with streptavidin-alexa 488 (Invitrogen). Blood vessels were detected using a mouse anti-human CD31 antibody (eBioscience) followed by detection with an anti-mouse alexa 594 antibody (Invitrogen). Counterstaining for cell nuclei was performed with DAPI (eBioscience).

(ii) Freshly frozen human biopsy samples of Ulcerative Colitis were stained as described in the following lines. In brief, purified antibodies in human IgG1 format were added at the final concentration of 2 μg/ml to the sections. Detection of the primary antibody was performed rabbit anti human-IgG (DAKO) and signals were revealed with a goat anti-rabbit Alexa 488. Positive control was performed by staining blood vessels with an antibody (eBioscience, 1:100) specific for CD31, an endothelial cell marker, the signal revealed with goat anti-mouse Alexa-594 (Invitrogen). Further positive control was performed by counterstaining for cell nuclei was performed with DAPI (eBioscience). Sections were mounted with fluorescent mounting medium (DAKO) followed by analysis using an Axioskop2 microscope with a 10× objective (Carl Zeiss AG, Jena, Germany).

Autoradiography

After 2 weeks of acclimatization, 8-week-old specific pathogen-free female C57BL/6 mice (Janvier Labs) received 3.0% (wt/vol) DSS (40,000 g/mol, TdB Consultancy) in drinking water ad libitum. Five days later, DSS water was replaced by water supplemented with 5% glucose and 0.25% NaHCO₃ for 7 days, followed by nonsupplemented (i.e., normal) water. Body weight and disease score were assessed daily. On day 12 after colitis induction, mice received 10 μg of radiolabelled IgG antibody intravenously through the lateral tail vein. Forty-eight hours later, the complete intestinal tract (comprehensive of small intestine and colon) was taken, washed with saline, and exposed to a phosphorimaging plate (Fujifilm Holdings Corp, Tokyo, Japan). All autoradiographic pictures obtained in one experiment were processed with identical parameters.

Results

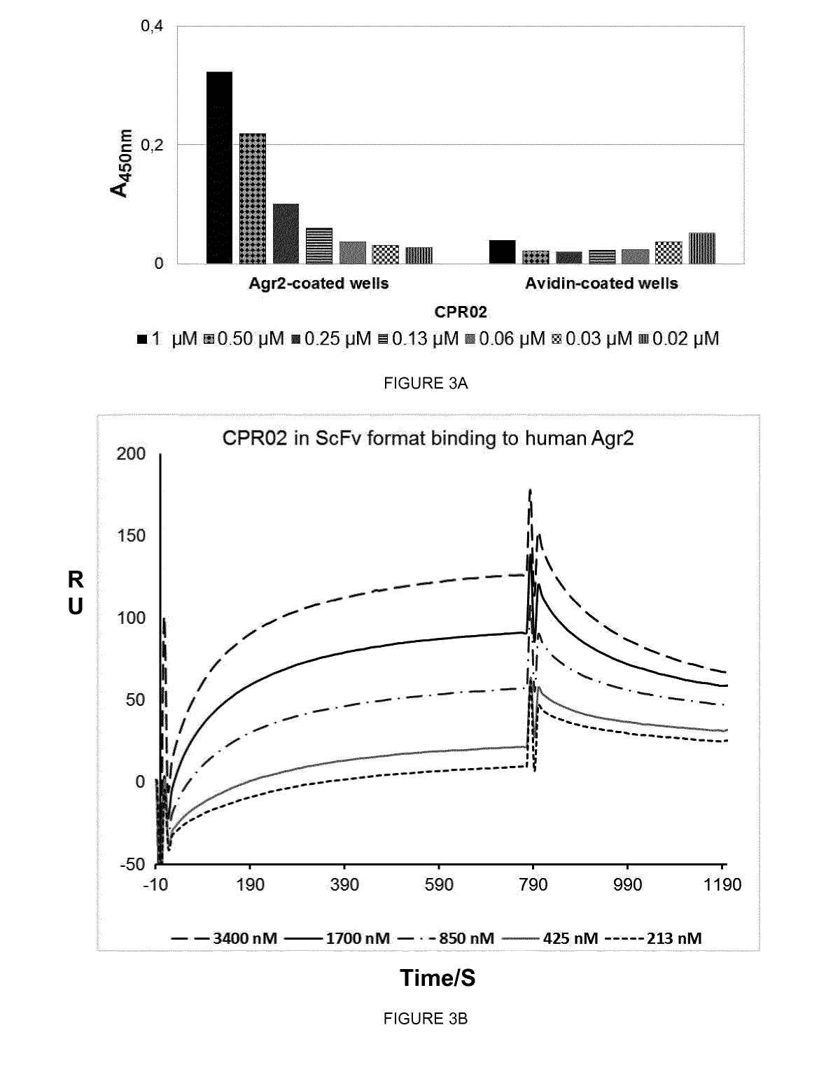

One scFv (CPR02) was isolated from the antibody library after three rounds of panning against residues 41-175 of recombinant human Agr2. ScFv (CPR02) was able to bind the recombinant human Agr2 protein in an ELISA (FIG. 3A). The scFv antibody did not bind to wells coated with avidin alone, confirming that the ELISA signal observed was due to binding to Agr2. A more precise affinity constant determination was performed by BiaCore analysis with the monomeric scFv(CPR02) on a human Agr2 coated chip. The results are shown in FIG. 3B and demonstrate that monomeric scFv(CPR02) had an affinity (K_{D}) for human of 4.6×10⁻⁷ M.

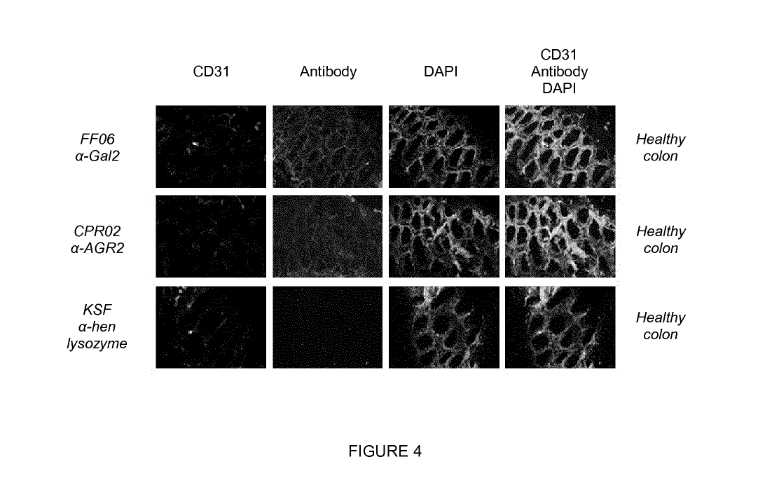

Immunofluorescence analysis on colon biopsy samples from a healthy donor was performed to verify the ability of CPR02 to recognize the human Agr2 antigen. The direct comparison of CPR02 to KSF (an antibody of irrelevant specificity in human as it recognizes hen egg lysozyme) reveals significant staining surrounding the crypts of the intestinal tissue (FIG. 4).

Immunofluorescence analysis on human biopsy samples of Ulcerative Colitis was performed. As shown in FIG. 9, a stronger intensity signal in the colonic crypts region can be detected in the sections stained with CPR02 when compared to the signal observed with an irrelevant antibody (Negative Control).

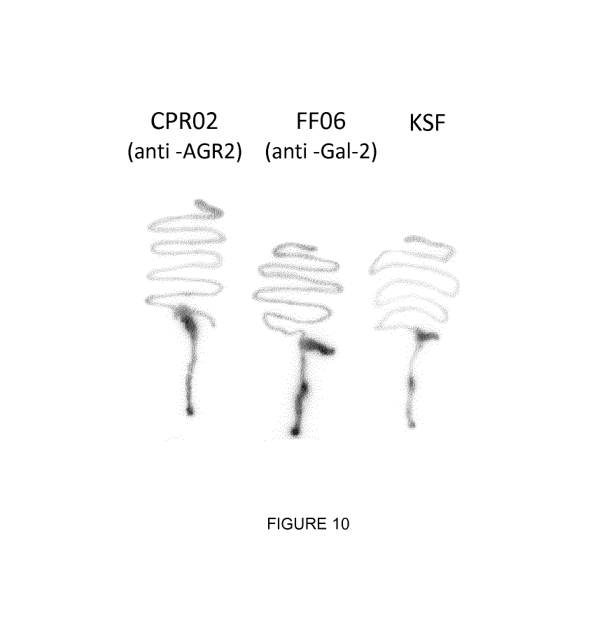

The autoradiography on the intestinal tract of a mouse having colitis has been performed in order to verify the ability of CPR02 to recognize the Agr2 antigen in vivo. A stronger intensity signal could be detected in the colon of the mouse injected with CPR02 when compared to the signal in a control mouse injected with an irrelevant antibody named KSF (FIG. 10).

### Example 4: Preparation and Characterization of Two New Antibodies Against Galectin-4

Materials and Methods

Antigen Preparation for Antibody Phage Display Selections

For the generation of fully human monoclonal antibodies against human galectin-4, a recombinant version of the full-length protein was produced in a mammalian expression system. Specifically, the DNA sequence encoding human LGALS4 (Uniprot P56470, residues 1-323) was expressed in CHO.S cells, and the protein was purified from the culture supernatant by affinity chromatography using a 6×Histidine tag appended to the C-terminus of the protein. The amino acid sequence of the recombinant Galectin-4 is set out in SEQ ID NO: 67.

The antigen was biotinylated by modification of surface lysine residues with an NHS-LC-biotin reagent (Thermo Fisher). The biotinylated antigen was used at a concentration of 0.12 μM for selections.

Antibody Selection

Biotinylated galectin-4 at a concentration of 0.12 μM was immobilized on streptavidin-coated M-280 dynabeads (Thermo Fisher). The coated beads were blocked with 2% w/v skimmed milk powder in phosphate-buffered saline (MPBS). The blocked beads were incubated with 10¹² phage particles from naïve phage libraries as disclosed in WO2010/028791 diluted in MPBS for 1.5 hour. Unbound phages were removed by washing with PBS+0.1% Tween-20 followed by washing with PBS alone. Bound phages were eluted by incubation of the beads with 800 μL of 100 mM trimethylamine for 5 min. The eluted phage solution was neutralized by addition of 0.4 mL of 1 M Tris-HCl, pH 7.4. Eluted phages were recovered through infection of exponentially growing E. coli TG1 cells.

Expression and Purification of scFv Antibody Fragments

ScFv antibody fragments from selected bacterial clones were produced by inoculating 10 mL of 2TY medium containing 100 μg/ml ampicillin and 1% v/v glucose with a single bacterial colony containing the scFv expression plasmid. The pre-culture was incubated at 37° C., 180 rpm for 4 hours until turbid, and then diluted 1:100 into 800 mL 2TY medium, 100 μg/ml ampicillin, 1% v/v glucose. The large scale culture was incubated at 37° C., 180 rpm until the culture reached mid-exponential phase, and protein expression was then induced with IPTG at a final concentration of 1 mM. The induced culture was incubated overnight at 30° C., 180 rpm. The scFv fragments were purified from the bacterial supernatant by affinity chromatography using Protein A Sepharose (Sino Biological Inc.) according to the manufacturer's instructions.

BiaCore Analysis

Binding of the purified scFvs to the recombinant galectin-4 antigen was confirmed by surface plasmon resonance experiments using a BiaCore T3000 instrument (GE Healthcare). Human galectin-4 was immobilized on CM5 sensor chip (GE Healthcare), and serial dilutions at concentrations between 500 nM and 15.6 nM of the purified scFv antibodies were injected over the surface.

Immunofluorescent Staining

Immunofluorescent staining was performed on the LS174T human colorectal adenocarcinoma cell line. LS174T cells were seeded onto 12 mm diameter glass coverslips at 30 000 cells/well. After 72 hours, cells were fixed in 2%-PFA at room temperature for 10 minutes. Coverslips were then washed with PBS and cells were permeabilized using 90% ice-cold methanol at −20° C. for 10 min. After fixation and permeabilization, coverslips were washed and blocked with 20% v/v fetal bovine serum in PBS for 30 min. After washing with PBS, scFvs were added at a concentration of 20 μg/mL in 3% w/v BSA/PBS solution containing the anti-myc tag antibody at a final concentration of 3 μg/ml, and incubated for 2 hours. Detection of bound scFvs was performed by 1 hour incubation with a goat anti-mouse Alexa594 antibody (Molecular probes) diluted 1:500 in 3% BSA/PBS. The nuclei were co-stained with DAPI. Coverslips were mounted with fluorescent mounting medium (Dako) and analyzed with an Axioskope2 mot plus microscope (Zeiss), using the 40× objective.

Results

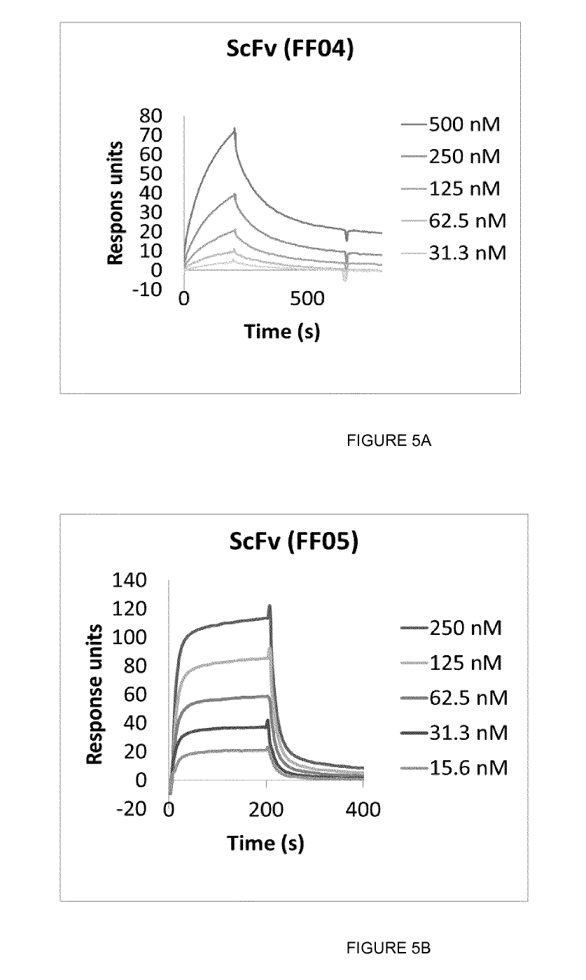

ScFv(FF04) and scFv(FF05) were isolated from an antibody library after three rounds of panning against human galectin-4. As depicted in FIG. 5, both scFv(FF04) and scFv(FF05) were able to bind the recombinant human galectin-4 protein as determined by surface plasmon resonance. The Biacore analysis demonstrates that scFv(FF04) had an affinity (K_{D}) for the human Galectin-4 of 2.44×10⁻⁷ M and scFv(FF05) had an affinity (K_{D}) for the human Galectin-4 of 2.0×10⁻⁶ M.

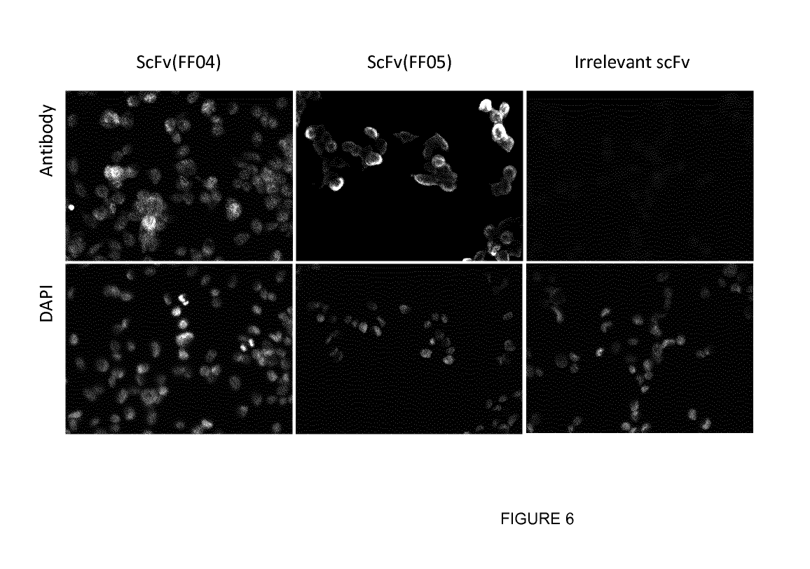

Immunofluorescence analysis on the human colorectal adenocarcinoma cell line LS174T was performed in order to verify the ability of scFv(FF04) and scFv(FF05) to recognize the natively expressed galectin-4 antigen. Both scFv(FF04) and scFv(FF05) showed strong cytoplasmic staining. The secondary and tertiary detection reagents did not elicit staining of the cells when used together with an irrelevant scFv as a negative control. The results are shown in FIG. 6.

### Example 5: Preparation and Characterization of a New Antibody Against Galectin-2

Materials and Methods

Antigen Preparation for Antibody Phage Display Selections

For the generation of fully human monoclonal antibodies against human galectin-2, a recombinant version of the full-length protein was produced in a bacterial expression system. Residues 1-132 of galectin-2 were expressed in a BL21-based expression strain and purified using a 6×Histidine tag appended to the C-terminus of the protein.

The antigen was biotinylated by modification of surface lysine residues with an NHS-LC-biotin reagent (Thermo Fisher). The biotinylated antigen was used at a concentration of 0.12 μM for the antibody selections. The amino acid sequence of the recombinant human galectin-2 is set out in SEQ ID NO: 92.

Antibody Selection Protocol

Biotinylated galectin-2 at a concentration of 0.12 μM was immobilized onto streptavidin-coated M-280 dynabeads (Thermo Fisher). The coated beads were blocked with 2% w/v skimmed milk powder in phosphate-buffered saline (MPBS). The blocked beads were incubated with 10¹² phage particles from naïve phage libraries as disclosed in WO2010/028791 and diluted in MPBS for 1.5 hour. Unbound phages were removed by washing with PBS+0.1% Tween-20 followed by PBS alone. Bound phages were eluted by incubation of the beads with 800 μL of 100 mM trimethylamine for 5 min. The eluted phage solution was neutralized by addition of 0.4 mL of 1 M Tris-HCl at pH 7.4. Eluted phages were recovered through infection of exponentially growing E. coli TG1 cells.

Expression and Purification of scFv Antibody Fragments

ScFv antibody fragments from selected bacterial clones were produced by inoculating 10 mL of 2TY medium containing 100 μg/ml ampicillin and 1% v/v glucose with a single bacterial colony containing the scFv expression plasmid. The pre-culture was incubated at 37° C., 180 rpm for 4 hours until turbid, and then diluted 1:100 into 800 mL 2TY medium, 100 μg/ml ampicillin, 1% v/v glucose. The large scale culture was incubated at 37° C., 180 rpm until the culture reached mid-exponential phase, and protein expression was then induced with IPTG at a final concentration of 1 mM. The induced culture was incubated overnight at 30° C., 180 rpm. The scFv fragments were purified from the bacterial supernatant by affinity chromatography using Protein A Sepharose (Sino Biological Inc.) according to the manufacturer's instructions.

ELISA

Binding of the purified scFvs to the recombinant galectin-2 antigen was confirmed by ELISA. Maxisorp plates (Nunc) were coated with avidin at 50 μg/mL in PBS for 6 hours at room temperature. Unbound avidin was removed by washing with PBS and the wells were coated with biotinylated galectin-2 at a final concentration of 0.1 μM overnight at 4° C. Unbound galectin-2 was removed by washing with PBS, and coated wells were blocked for 1 hour with 2% w/v skimmed milk powder in phosphate-buffered saline (MPBS). Serial dilutions of the purified scFv antibodies were prepared in MPBS at concentrations between 500 nM M and 4 nM. The anti-myc tag antibody 9E10 was added to the prepared antibody dilutions at a final concentration of 1 μg/ml and the mixture was incubated with the immobilized antigen for 1.5 hour at room temperature. Bound scFvs were detected using a 1:1000 dilution of HRP-conjugated anti-murine Fc (Sigma Aldrich) and the signal was developed using BM Blue POD substrate (Roche). The ELISA output was recorded at a wavelength of 450 nM, subtracting absorbance at 620 nM as a reference baseline.

Immunofluorescent Staining

(i) Immunofluorescent staining was performed on the LS174T human colorectal adenocarcinoma cell line. LS174T cells were seeded onto 12 mm diameter glass coverslips at 30 000 cells/well. After 72 hours, cells were fixed in 2%-PFA at room temperature for 10 minutes. Coverslips were then washed with PBS and cells were permeabilized using 90% ice-cold methanol at −20° C. for 10 min. After fixation and permeabilization, coverslips were washed and blocked with 20% v/v fetal bovine serum in PBS for 30 min. After washing with PBS, scFvs were added at a concentration of 20 μg/mL in 3% w/v BSA/PBS solution containing the anti-myc tag antibody at a final concentration of 3 μg/ml, and incubated for 2 hours. Detection of bound scFvs was performed by 1 hour incubation with a goat anti-mouse Alexa594 antibody (Molecular probes) diluted 1:500 in 3% BSA/PBS. The nuclei were co-stained with DAPI. Coverslips were mounted with fluorescent mounting medium (Dako) and analyzed with an Axioskope2 mot plus microscope (Zeiss), using the 40× objective.

(ii) Freshly frozen colon biopsy samples from a healthy donor were stained according to already published methods (Pfaffen et al., Eur J Nucl Med Mol Imagin (2010) 37: 1559-65). Briefly, purified biotinylated antibodies in IgG format were added at the final concentration of 2 μg/ml onto the sections. Detection of the primary antibody was performed with streptavidin-alexa 488 (Invitrogen). Blood vessels were detected using a mouse anti-human CD31 antibody (eBioscience) followed by detection with an anti-mouse alexa 594 antibody (Invitrogen). Counterstaining for cell nuclei was performed with DAPI (eBioscience).

(iii) Freshly frozen human biopsy samples of Ulcerative Colitis were stained as follow. Purified antibodies in human IgG1 format were added at the final concentration of 2 μg/ml to the sections. Detection of the primary antibody was performed rabbit anti human-IgG (DAKO) and signals were revealed with a goat anti-rabbit Alexa 488. Positive control was performed by staining blood vessels with an antibody (eBioscience, 1:100) specific for CD31, an endothelial cell marker, the signal revealed with goat anti-mouse Alexa-594 (Invitrogen). Further positive control was performed by counterstaining for cell nuclei was performed with DAPI (eBioscience). Sections were mounted with fluorescent mounting medium (DAKO) followed by analysis using an Axioskop2 microscope with a 10× objective (Carl Zeiss AG, Jena, Germany).

Autoradiography

After 2 weeks of acclimatization, 8-week-old specific pathogen-free female C57BL/6 mice (Janvier Labs) received 3.0% (wt/vol) DSS (40,000 g/mol, TdB Consultancy) in drinking water ad libitum. Five days later, DSS water was replaced by water supplemented with 5% glucose and 0.25% NaHCO₃ for 7 days, followed by nonsupplemented (i.e., normal) water. Body weight and disease score were assessed daily. On day 12 after colitis induction, mice received 10 μg of radiolabelled IgG antibody intravenously through the lateral tail vein. Forty-eight hours later, the complete intestinal tract (comprehensive of small intestine and colon) was taken, washed with saline, and exposed to a phosphorimaging plate (Fujifilm Holdings Corp, Tokyo, Japan). All autoradiographic pictures obtained in one experiment were processed with identical parameters.

Results

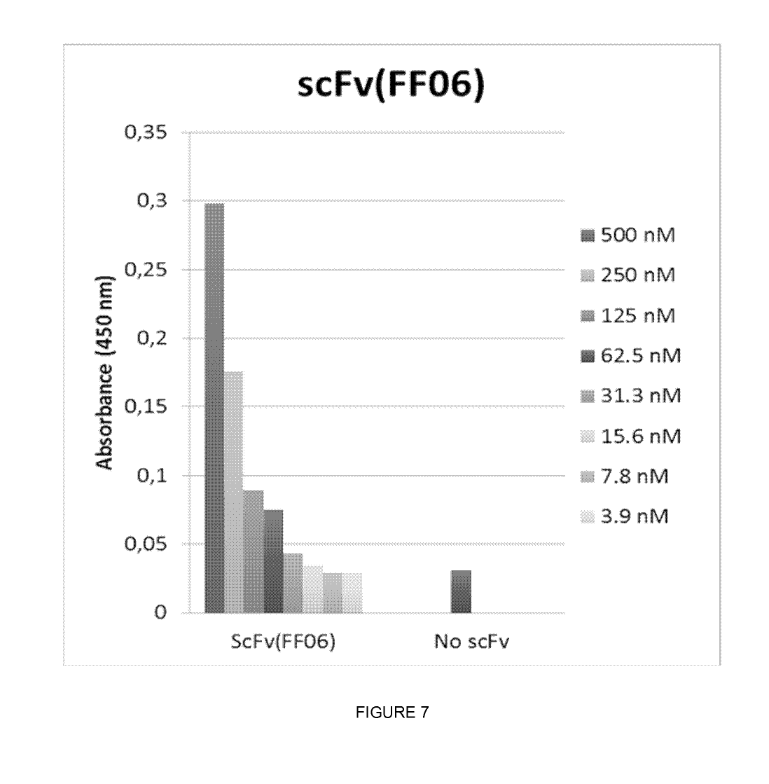

ScFv(FF06) was isolated from the antibody library after two rounds of panning against human galectin-2. ScFv(FF06) was able to bind the recombinant human galectin-2 protein in ELISA experiments as shown in FIG. 7. The scFv antibody did not bind to wells coated with avidin alone, confirming that the ELISA signal observed was due to binding to galectin-2. This result was confirmed by BiaCore (data not shown).

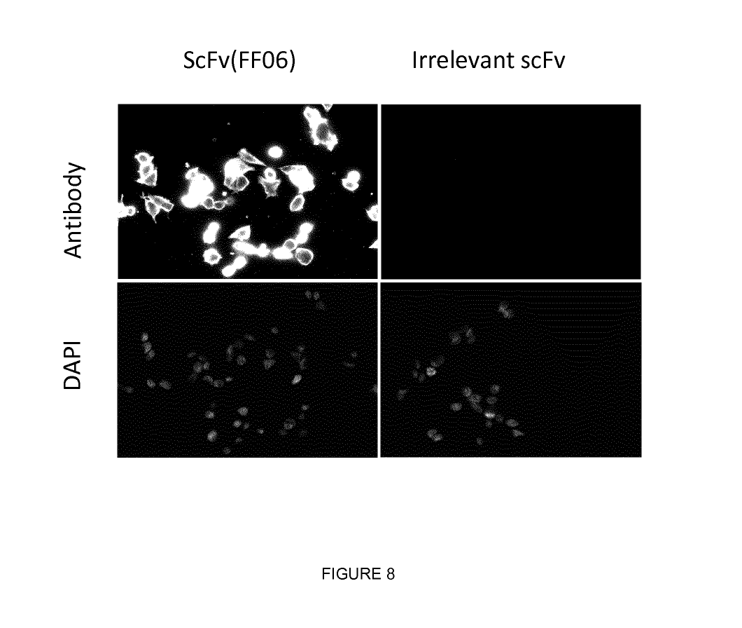

Immunofluorescence analysis on the human colorectal adenocarcinoma cell line LS174T was performed in order to verify the ability of scFv(FF06) to recognize natively expressed galectin-2 antigen. ScFv(FF06) showed strong cytoplasmic staining (FIG. 8). The secondary and tertiary detection reagents did not elicit staining of the cells when used together with an irrelevant scFv.

Immunofluorescence analysis on the colon biopsy samples from a healthy donor was performed. The direct comparison of FF06 to KSF (antibody of irrelevant specificity in human as it recognizes hen egg lysozyme) reveals significant staining surrounding the crypts of the intestinal tissue (FIG. 4).

Immunofluorescence analysis on human biopsy samples of Ulcerative Colitis was performed. As shown in FIG. 9, a stronger intensity signal in the colonic crypts region can be detected in the sections stained with FF06 when compared to the signal observed with an irrelevant antibody (Negative Control).

The autoradiography on the intestinal tract of a mouse having colitis has been performed in order to verify the ability of FF06 to recognize the galectin-2 antigen in vivo. A stronger intensity signal could be detected in the colon of the mouse injected with FF06 when compared to the signal in a control mouse injected with an irrelevant antibody named KSF (FIG. 10).

Sequence listing

Murine protein sequences
Amino acid sequence of Galectin-2 from mus musculus (SEQ ID NO: 1)
MSEKFEVKDLNMKPGMSLKIKGKIHNDVDRFLINLGQGKETLNLHFNPRFDESTIVCNTS
EGGRWGQEQRENHMCFSPGSEVKITITFQDKDFKVTLPDGHQLTFPNRLGHNQLHYLSMG
GLQISSFKLE
Amino acid sequence of IgGFc-binding protein (Fcgbp) from mus musculus
(SEQ ID NO: 2)
MGSPWKWWALWAGATLLWALLTPAEASCQGIQCASGQRCQMVSGKARCVAESTAVCRAQG
DPHYTTFDGRRYDMMGTCSYTMAELCGSDETLPAFSVEAKNEHRGSRQVSYVGLVTVYAY
SHSVSLVRGEIGFVRIDNQRSRLPASLSEGRLRVHKSGTRGVIEMDFGLVVTYDWDGQLT
LSLPKRFQDQVCGLCGNYNGDPADDFLTPDLDQAPDALEFANSWKLDDGDYLCDDGCHNS
CPSCTPGQTQHYKGDRLCGMLTLSTGPFSACHEFLDPKPFLDDCVFDLCVTGGERLSLCR
SLSAYAQACVELGVTLENWRLPASCPMSCPANSCYDPCSPACPPSCNSEAVPTNCSSRPC
VEGCVCLPGFVASGGDCVPVSSCGCIYQGRLLAPGQEVFDDDRCRRRCTCDGATQKVTCR
DTTGCPSGERCNVQNGLLGCYPDNFASCQASGDPHYVSFDGKRFDFMGTCTYLLVGSCGQ
NAALPAFKVLVENEHRGSQTVSYTRAVRVVAHGVEVAVRRENPGRVLIDGVLQYLPFQAA
GGKIQVYRQGNDAIVSIDFGLTVTYNWDAHVTAKVPSSYAKDVCGLCGNFNGNPDDDLAL
KGGGQASNVLDFGNSWQEEIIPGCGATEPGDCPQLDSLVTQQIQDKKECGILADPEGPFR
ECHKLLNPQGAIRDCVYDLCLLPGQSGPLCDALAAYAAACQAAGGTVHPWRSEELCPLTC
PPNSHYEQCSYGCPLSCGDLPVQGGCGSECREGCVCNEGFALSGESCVPLASCGCVHEGA
YHAPGETFYPGPGCDSLCHCEEGGLVSCEPSSCGPQEACQPSNGVLGCVAVGTTTCQASG
DPHYVTFDGRRFDFMGTCVYVLAQTCGNRPGLHQFTVLQENEAWGNGKVSVTKVITVLVA
NYTLRLEQSQWKVKVNGVDTKLPVMLDGGKIRVSQHGSDVVIETDFGLRVAYDLVYYVRV
TIPGNYYKQMCGLCGDYNGDPKDDFQKPDGSQTTDPSDFGNSWEEAVPDSPCAPVPPCTG
DDCDTECSPELQDKYHGEQFCGLLTSPTGPLAACHKLLDPQGPLQDCVFDLCLGGGNQSI
LCNIIHAYVSACQAAGGQVEPWRTETFCPMECPPHSHYEVCADTCSLGCWALNTPQQCPE
GCAEGCECDSGFLYNGKACVPIEQCGCYHNGVYYEPEESVLIENCQQHCVCQPGKGMMCQ
DHSCKPGQVCEPSGGVLTCVTKDPCHGITCRPQETCKVQGGEGVCVPNYNSTCWLWGDPH
YNSFDGWSFDFQGTCNYLLAGTLCPGVNAEGLTPFTVTTKNENRGSPAVSYVRQVTVTTL
NTNISIHKNEIGKVRVNGVLMALPVYLAGGRISVINGGSKAVLETDFGLQVTYDWNWRVD
VTLPSSYHGAVCGLCGNMDKNHQNDQVFPNGTMAPSIPTWGGSWQVPGWDPLCWHECQGS
CPTCPEDRVEEYEGPGFCGPLAPGTGSPFTSCHAHVPPESFFKGCVLDVCLGGGSKDILC
QALAAYAAACQAAGIKIEDWRTQAGCEITCPDNSHYELCGPPCPASCPPPARHTAPTVCD
GPCVEGCQCDEGFVLSADQCVPLDGGCGCWVNGTYYEAGTEFWADTTCSKRCHCGPGGDS
LVCKPASCGLGEECALLPSGEIGCQPTSITECQAWGDPHYTTLDGHRFDFQGTCEYLLSA
PCHEPPTGTEYFNVTVANEHRGSQAVSYTRSVTLQIYGLSLTLSAQWPRKLQVNGEFVAL
PFHLDQKLSVYISGADVVVNTASGVSLAFDGDSFVRLRVPAAYAGTLCGLCGNYNKNPND
DLTAVGGKPEGWKVGGAPGCDQCEPEPCPKPCTPEEQEPFRGPDACGIITAPEGPLAPCH
SLVPPTQYFEACLLDACQVQGHPGGLCPAIATYVAACQAAGAQLGEWRKPDFCPLQCPAH
SHYQLCGDSCPVSCPSLSAPVGCETICREGCVCDAGFVLSGDTCVPVGQCGCLYQGRYYV
LGATFYPGPECERLCECGPDGQVTCQEGADCEPYEECRIENGVQACHPTGCGHCLANGGL
HYVTLDGRVYDLHGSCSYVLASVCHPKPGDEEFSIVLEKNSAGDPQRVVVTVAGQVVGLA
RGPQVTVDGEVVTLPVATGHVSVTAEGRNIVLQTNKGMKVLFDGDAHILMSIPSSFRGRL
CGLCGNFNGNWSDDFVLPSGAVAPNVEAFGTAWRAPGSSLGCGEGCGPQGCPVCLAEETQ
AYEKNDACGKIRDPHGPFAACHKVLSPLEYFRQCVYDMCAHKGDKAYLCRSLAAYTAACQ
AAGAAVKPWRTDSVCPLQCPAHSHYSICTRSCQGSCAALSGLTGCTTRCFEGCECDDHFL
LSHGVCIPAQDCGCVHNGQYMPVNSSLMSSDCSERCFCSPNNGLTCHEAGCPSGHVCEIQ
AGVRECQAARGLCSISVGANLTTFDGAHNAISSPGVYELSSRCPGLQKNVPWYRVLADVQ
PCHNNDKIVSKVHIFFQDGLVTVIPSKGAWVNGLRVDLPATVLTSVSVRRMPDGSMLVHQ
KAGVTVWLGKDGLLDVMVGDDLAAMLCGACGNFDGDQTNDAYGSQGKTP1EKWRAQDFSP
CSN
Amino acid sequence of galectin-4 from mus musculus (SEQ ID NO: 3)
MAYVPAPGYQPTYNPTLPYKRPIPGGLSVGMSVYIQGMAKENMRRFHVNFAVGQDDGADV
AFHFNPRFDGWDKVVFNTMQSGQWGKEEKKKSMPFQKGKHFELVFMVMPEHYKVVVNGNS
FYEYGHRLPVQMVTHLQVDGDLELQSINFLGGQPAAAPYPGAMTIPAYPAGSPGYNPPQM
NTLPVMTGPPVFNPRVPYVGALQGGLTVRRTIIIKGYVLPTARNFVINFKVGSSGDIALH
LNPRIGDSVVRNSFMNGSWGAEERKVAYNPFGPGQFFDLSIRCGMDRFKVFANGQHLFDF
SHRFQAFQMVDTLEINGDITLSYVQI
Amino acid sequence of mucin-2 (Fragments) from mus musculus
(SEQ ID NO: 4)
MGLPLARLVAACLVLALAKGSELQKEARSRNHVCSTWGDFHYKTFDGDVYRFPGLCDYNF
ASDCRDSYKEFAVHLKRGLGEAGGHSQIESILITIKDDTIYLTHKLAVVNGAMVSTPHYS
SGLLIEKNDAYTKVYSRAGLSLMWNREDALMVELDSRFQNHTCGLCGDFNGMQTNYEFLS
EEGIQFSAIEFGNMQKINKPEVQCEDPEAVQEPESCSEHRAECERLLTSAAFEDCQTRVP
VESYVRACMHDRCQCPKGGACECSTLAEFSRQCSHAGGRPENWRTASLCPKKCPNNMVYL
ESSSPCVDTCSHLEVSSLCEEHYMDGCFCPEGTVYDDITGSGCIPVSQCHCKLHGHLYMP
GQEFTNDCEQCVCNAGRWVCKDLPCPETCALEGGSHITTFDGKKFTFHGDCYYVLTKSEH
NDSYALLGELASCGSTDKQTCLKTVVLLTDDKKNVVAFKSGGSVLLNEMEVTLPHVAASF
SIFQPSSYHIVVNTKFGLRLQIQLLPVMQLFVTLDQAAQGQVQGLCGNFNGLESDDFMTS
GGMVEATGAGFANTWKAQSSCHDKLDWLDDPCSLNIETNYAEHWCSLLKRSETPFARCHL
AVDPTEYYKRCKYDTCNCQNNEDCMCAALSSYARACAAKGVMLWGWRERVCNKDVHACPS
SQIFMYNLTTCQQTCRSLSEGDSHCLKGFAPVEGCGCPDHTFMDEKGRCVPLAKCSCYHH
GLYLEAGDVILRQEERCICRNGRLQCTQVKLIGHTCQYPKILVDCNNLTALAVRKPRPTS
CQTLVAGYYHTECISGCVCPDGLLDDGRGGCVEEDKCPCIHNKDLYSSGESIKLDCNNTC
TCQKGRWECTRYACHSTCSIYGSGHYITFDGKHYDFDGHCSYVAVQDYCGQNSTGSFSII
TENVPCGTTGVTCSKAIKIFIGGTELKLVDKHRVVKQLEEGHHVPYITREVGQYLVVEAS
SGIIVIWDKKTTIFIKLDPSYKGTVCGLCGNFDDQTKNDFTTRDHMVVTSELDFGNSWKE
ASTCPDVSHNPDPCSLNPHRRSWAEKQCSIIKSRVFKVCHSKVDPTVFYEACVHDSCSCD
TGGDCDCFCSAVASYAQECTKAEACVFWRTPDLCPIFCDYYNPPDECEWHYEPCGNRSFE
TCRTLNGIHSNISVSYLEGCYPRCPEDRPIYDEDLKKCVTGDKCGCYIEDTRYPPGGSVP
TDEICKSCTCTNTSKIECHPDEGKILNMTQDGIFCYWEFCGPNGTVGQHFNICGSSTAIP
STTTSFTTISTPISTTPISTTITTTTVTMTTEQVPCCFWSDWINKYHPTKENGGDRETFT
HVCSAPEDIECRAATDPKLSWEELGQKVQCNVSTGLICNNEDQYGIGEFELCYDYEIRVN
CCYPMEYCTPSTISPTTSTTTLSTTPPTSSPTTLPTSSPVTSSATLPTTSSITSTISPTT
SPSTATQTISVTTSQTSSSATPPNSSPTSSATTSPTTSSGTSTATSPSTSPTTSSTFTTP
PSTTCIDDCKWTGWLDSGKPTYDIKSGDFELIKGVCEPHWEVQNISCRAVMHSNIPLDQL
GQIVVCNKEVGLVCKNEDQEIGGIIPMRMCLNYEINVYCCNPICFTSTPSSTTTETPTTT
STTKTSILTSTTTQTPSPSPTTTVTPTPAPTTTQIPTSTSTTTQTTTPTPITETSTPTST
ISQTPSPASTTTVTPATTSTTTETSTSTSTTTQTTSPTPTVTETSTPRSTTTQTPSPVPT
TTVTSTPTPTIGETTTPKRPPSTSTPTSFTVPTETTTQTRPLSTTPTTLETTRTSSWGTF
SSTSPITSPSTVWTHTETQVTCCVLNEMFYGPGELVYNSTHGGTCFYVNCSLDCHLQFFN
WSCPSTPSTPTPSTPTPTPSQTTTPSTTSSKSTPSTPQSTSPKSTLSTPTKTTPYGCPDF
DPPRQVNETWWLCNCTMAICNHDNVVEIVPLKCDPPPMPTCANGLKPVRVPDADNCCWHW
ECDCYCTGWGDPHFVTFDGLYYSYQGNCTYVLVEEITPTVDNFGVYIDNYHCDANDKVSC
PRTLIVRHETQEVQIKTVRMMPIEVEVQVNKQLVALPYKKYGLEVYESGINIVVNISRLE
AKISYNGLSFSIRLPYKLFVNNTKGQCGTCTNNTADDCILPSGKIISDCEIAADEWLVND
PSKPHCPHKGLTTKRPATTTPGLSLNNCTVSPVCHLIMDSLFSQCHAFVPPKHYYEACLF
DSCYVPGSNMECASVQAYATLCAKEGVCIDWRNHTQGVCSVKCPPHKQYQACGPEEEPTC
QPSSSQNSTLLVEGCFCPEGTTKFAPGYDVCVKTCGCVGPDNVPREFGEHFEFDCKDCVC
REGGSGIVCQPKKCSGGNQTTCEEDGTYLVVETNPDDKCCNITSCKCDTKRCKAERPTCL
LGFEVKTEIVPGKCCPVYSCVPKGVCVHQNAEYQPGSPVYSNKCQDCVCTNILDNSTQLN
VISCTHVPCNISCSSGFELVDVPGECCKKCQQTHCIIEGPKQQYIILKPGEIHKNPSNKC
TFFSCMKINNQLISSVSNITCPDFNPSDCVSGSITYMPNGCCKTCIPQNQTRVPCSAVSV
MKEISYNGCTKNISMNYCFGSCGTFAMYSAQVQGLDHRCSCCKEEKTSVRSVTLECPDGS
ELSHTYTHIESCLCQDTVCGLPQAQQVRTRRSSPRFLGRK
Amino acid sequence of anterior gradient protein 2 homolog from mus
musculus (SEQ ID NO: 5)
MEKFSVSAILLLVAISGTLAKDTTVKSGAKKDPKDSRPKLPQTLSRGWGDQLIWTQTYEE
ALYRSKTSNRPLMVIHHLDECPHSQALKKVFAEHKEIQKLAEQFVLLNLVYETTDKHLSP
DGQYVPRIVFVDPSLTVRADITGRYSNRLYAYEPSDTALLYDNMKKALKLLKTEL
Amino acid sequence of cadherin-17 from mus musculus (SEQ ID NO: 6)
MVSAQLHFLCLLTLYLTCGYGEEGKFSGPLKPMTFSIFEGQEPSQVIFQFKTNPPAVTFE
LTGETDGIFKIEKDGLLYHTRALDRETRAVHHLQLAALDSHGAIVDGPVPITIEVKDIND
NRPTFLQSKYEGSVRQNSRPGKPFMYVNATDLDDPATPNGQLFYQIVIQLPQINDVMYFQ
IDSKTGAISLTPEGSQELDPVKNPSYNLVVSVKDMGGQSENSFSDTTYVDISIRENIWKA
PEPVEIRENSTDPHPIKITQVQWNDPGAQYSLVNKEKLSPFPFSIDQEGNIYVTQALDRE
EKNSHVFFATAKDENGKPLAYPLEIYVKVIDINDNPPTCLSPVTVFEVQENEPLGNSIGI
FEAHDMDEANNINSILKYKLVDQTPKVPSDGLFLIGEYEGKVQLSKQSLKKQDSPQYNLS
IEVSDVDFKTLCYIQVNVIDINDQIPIFETSNYGSKTLSEDTAIGSTILIIQATDADEPF
TGSSKILYKIVQGDTEGRLEVVTDPTTNAGYVKIKKPLDFETQPVSSIVFQAENPEPLVK
GIEYNASSFASFELIVTDVNEVPVFPQRIFQANVSEDAAVGSRVGNVTARDPEGLTVSYS
LKGNMRGWLKIDSVTGEIFSAAPLDRETESVYRVQVVATEVGGSSLSSTADFHLVLTDVN
DNPPRLAKDYTGLFFCHPLSAPGSLIFEVTDDDQQSLRRPKFTFALGREGLQSDWEVSKI
NGTHARLSTRHTRFEEQVYNIPIRINDGGQPPMEGTVFLPVTFCQCVEGSCFRPAGRQDG
IPTVGMAVGILLTTFLVIGIILAVVFIRMRKDKVENPQSPENKPLRS
Amino acid sequence of zymogen granule membrane protein 16 from mus
musculus (SEQ ID NO: 7)
MLAVALLVLLCASASANSIQSRTSSYSGEYGGKGGKRFSHSGNQLDGPITAFRIRVNRYY
IVGLQVRYGTVWSDYVGGTQGDLEEIFLHPGESVIQVSGKYKSYVKQMIFVTDKGRYLPF
GKASGTSFNAVPLHPNTVLRFISGRSGSAIDSISLHWDTYPSHCNTC
Amino acid sequence of metal transporter CNNM4 from mus musculus (SEQ
ID NO: 8)
MAPGGGGGRRDGWPARGRLLLAALLLLWTRAASGQSSPQQSVILGMRLASCNKSCGMNPD
GIIFVSEGSTVNLRLYGHSLGDISSNLISFTEVDDAEAVHNSTNCLELTKDLVVQRLVNV
SRGNTSGMLVVITKFLRRSENMKLYALCTRPRADGPWTRWTDKDSLLFMVEEHGRFLPLW
LHILLVMVLLVLSGIFSGLNLGLMALDPMELRIVQNCGTEKERKYARKIEPIRRKGNYLL
CSLLLGNVLVNTSLTILLDNLIGSGIMAVASSTIGIVIFGEILPQALCSRHGLAVGANTI
VLTKVFMLLTFPLSFPISKLLDFVLGQEIRTVYNREKLMEMLKVTEPYNDLVKEELNMIQ
GALELRTKTVEDIMTQLHDCFMIRSDAILDFNTMSEIMESGYTRIPVFEDEQSNIVDILY
VKDLAFVDPDDCTPLKTITRFYNHPVHFVFHDTKLDAMLEEFKKGKSHLAIVQKVNNEGE
GDPFYEVLGLVTLEDVIEEIIKSEILDESDMYTDNRTRKRVSVKNKRDFSAFKDTDNELK
VKISPQLLLAAHRFLATEVPQFSPSLMSEKILLRLLKYPDVIQELRFNEHNRYCVRHYLY
TRNKPADCFVLILQGKVEVEAGKENMKFETGAFSYYGTMALSVAPPDRSPALPTPLSRSA
SLSYPDRNTDLTSTSLAGSNQFGSCILGQYVSDFSVRALTDLQYIKITRQQYQNGLMASR
MDNSPQPTFDGCATCSENFMERPELPPVDETTTLLNERNSLLHRASEEETI
Amino acid sequence of amiloride-sensitive amine oxidase [copper-
containing] from mus musculus (SEQ ID NO: 9)
MSLAFGWAAVILLLQTADTASAVTTPHDKARIFADLSPQEIKAVHSFLMSRKELGLESSK
NLTLAKNSVFLIEMLLPKKKNVLKFLDEGRKSPVREARAIIFFGAQDHPNVTEFAVGPLP
RPCYVQALSPRPGHHLSWSSRPISTAEYDLLYHMLNRAITPLHQFFLDTTGFSFLGCDDR
FLTFTDVAPRGVESGQRRSWLIVQRYVEGYFLHPTGLEILVDHSSTDVQDWRVEQLWYNG
KFYNSPEELAQKYAVGEVEAVVLEEVVLEDPLPGATEQPPLFSSYKPRGEFHTPVTVAGP
HVVQPSGPRYKLEGNVVLYGDWSFSYRLRSSSGLQIFNVLFGGERVAYEVSVQEAVALYG
GHTPAGMQTKYIDVGWGLGSVTHELAPGIDCPETATFLDAFHYYDSDGPVLYPRALCLFE
MPTGVPLRRHFDSNFKGGFNFYAGLKGYVLVLRTTSTVYNYDYIWDFIFYPNGVMETKMH
ATGYVHATFYTPEGLRHGTRLQTHLLGNIHTHLVHYRVDLDVAGTKNSFRTLKTKLENIT
NPWSPSHSLVQPTLEQTQYSHEHQAAFRFGQTLPKYLLFSSPQKNRWGHRRSYRLQIHSM
AEQVLPPGWQEERAVTWARYPLAVTKYRESERYSSSLYNQNDPWDPPVVFEEFLRNNENI
ENEDLVAWVTVGFLHIPHSEDVPNTATPGNCVGFLIRPFNFFEEDPSLASRDTVIVWPQD
NGLNHVQRWIPENRDCLVSPPFSYNGTYKPV
Amino acid sequence of Hephaestin from mus musculus (SEQ ID NO: 10)
MKAGHLLWALLLMHSLWSIPTDGAIRNYYLGIQDMQWNYAPKGRNVITNQTLNNDTVASS
FLKSGKNRIGSSYKKTVYKEYSDGTYTEEIAKPAWLGFLGPLLQAEVGDVILIHLKNFAS
RPYTIHPHGVFYEKDSEGSLYPDGSSGYLKADDSVPPGGSHVYNWSIPESHAPTEADPAC
LTWIYHSHVDAPRDIATGLIGPLITCKRGTLDGNSPPQRKDVDHNFFLLFSVIDENLSWH
LDDNIATYCSDPASVDKEDGAFQDSNRMHAINGFVFGNLPELSMCAQKHVAWHLFGMGNE
IDVHTAFFHGQMLSIRGHHTDVANIFPATFVTAEMVPQKSGTWLISCEVNSHLRSGMQAF
YKVDSCSMDPPVDQLTGKVRQYFIQAHEIQWDYGPIGYDGRTGKSLREPGSGPDKYFQKS
SSRIGGTYWKVRYEAFQDETFQERVHQEEETHLGILGPVIRAEVGDTIQVVFYNRASQPF
SIQPHGVFYEKNSEGTVYNDGTSHPKVAKSFEKVTYYWTVPPHAGPTAQDPACLTWMYFS
AADPTRDTNSGLVGPLLVCKAGALGADGKQKGVDKEFFLLFTVFDENESWYNNANQAAGM
LDSRLLSEDVEGFQDSNRMHAINGFLFSNLPRLDMCKGDTVAWHLLGLGTETDVHGVMFE
GNTVQLQGMRKGAVMLFPHTFVTAIMQPDNPGIFEIYCQAGSHREEGMQAIYNVSQCSSH
QDSPRQHYQASRVYYIMAEEIEWDYCPDRSWELEWHNTSEKDSYGHVFLSNKDGLLGSKY
KKAVFREYTDGTFRIPRPRSGPEEHLGILGPLIRGEVGDILTVVFKNKASRPYSIHAHGV
LESNTGGPQAAEPGEVLTYQWNIPERSGPGPSDSACVSWIYYSAVDPIKDMYSGLVGPLV
ICRNGILEPNGGRNDMDREFALLFLIFDENQSWYLKENIATYGPQESSHVNLKDATFLES
NKMHAINGKLYANLRGLTVYQGERVAWYMLAMGQDTDIHTVHFHAESFLYQNGQSYRADV
VDLFPGTFEVVEMVASNPGTWLMHCHVTDHVHAGMETIFTVLSHEEHFSTMTTITKEIGK
AVILRDIGGDNVKMLGMNIPIKDVEILSSALIAICVLLLLIALALGGVVWYQHRQRKLRR
NRRSILDDSFKLLSLKQ
Amino acid sequence of glutamine-fructose-6-phosphate aminotransferase
[isomerizing] 1 from mus musculus (SEQ ID NO: 11)
MCGIFAYLNYHVPRTRREILETLIKGLQRLEYRGYDSAGVGLDGGNDKDWEANACKIQLI
KKKGKVKALDEEVHKQQDMDLDIEFDVHLGIAHTRWATHGEPNPVNSHPQRSDKNNEFIV
IHNGIITNYKDLKKFLESKGYDFESETDTETIAKLVKYMYDNWESQDVSFTTLVERVIQQ
LEGAFALVFKSVHFPGQAVGTRRGSPLLIGVRSEHKLSTDHIPILYRTARTQIGSTWWGS
QAERGKDKKGSCGLSRVDSTTCLFPVEEKAVEYYFASDASAVIEHTNRVIFLEDDDVAAV
VDGRLSIHRIKRTAGDHPGRAVQTLQMELQQIMKGNFSSFMQKEIFEQPESVVNTMRGRV
NFDDYTVNLGGLKDHIKEIQRCRRLILIACGTSYHAGVATRQVLEELTELPVMVELASDF
LDRNTPVFRDDVCFFISQSGETADTLMGLRYCKERGALTVGITNTVGSSISRETDCGVHI
NAGPEIGVASTKAYTSQFVSLVMFALMMCDDRISMQERRKEIMLGLKRLPDLIKEVLSMD
DEIQKLATELYHQKSVLIMGRGYHYATCLEGALKIKEITYMHSEGILAGELKHGPLALVD
KLMPVIMIIMRDHTYAKCQNALQQVVARQGRPVVICDKEDTETIKNTKRTIKVPHSVDCL
QGILSVIPLQLLAFHLAVLRGYDVDFPRNLAKSVTVE
Amino acid sequence of polymeric immunoglobulin receptor from mus
musculus (SEQ ID NO: 12)
MRLYLFTLLVTVFSGVSTKSPIFGPQEVSSIEGDSVSITCYYPDTSVNRHTRKYWCRQGA
SGMCTTLISSNGYLSKEYSGRANLINFPENNTFVINIEQLTQDDTGSYKCGLGTSNRGLS
FDVSLEVSQVPELPSDTHVYTKDIGRNVTIECPFKRENAPSKKSLCKKTNQSCELVIDST
EKVNPSYIGRAKLFMKGTDLTVFYVNISHLTHNDAGLYICQAGEGPSADKKNVDLQVLAP
EPELLYKDLRSSVTFECDLGREVANEAKYLCRMNKETCDVIINTLGKRDPDFEGRILITP
KDDNGRFSVLITGLRKEDAGHYQCGAHSSGLPQEGWPIQTWQLFVNEESTIPNRRSVVKG
VTGGSVAIACPYNPKESSSLKYWCRWEGDGNGHCPVLVGTQAQVQEEYEGRLALFDQPGN
GTYTVILNQLTTEDAGFYWCLTNGDSRWRTTIELQVAEATREPNLEVTPQNATAVLGETF
TVSCHYPCKFYSQEKYWCKWSNKGCHILPSHDEGARQSSVSCDQSSQLVSMTLNPVSKED
EGWYWCGVKQGQTYGETTAIYIAVEERTRGSSHVNPTDANARAKVALEEEVVDSSISEKE
NKAIPNPGPFANEREIQNVGDQAQENRASGDAGSADGQSRSSSSKVLFSTLVPLGLVLAV
GAIAVWVARVRHRKNVDRMSISSYRTDISMADFKNSRDLGGNDNMGASPDTQQTVIEGKD
EIVTTTECTAEPEESKKAKRSSKEEADMAYSAFLLQSSTIAAQVHDGPQEA
Amino acid sequence of sulfate transporter from mus musculus (SEQ ID
NO: 13)
MSSENKEQHDLSPRDLPEEAFGFPSELPLETQRRSGTDLRQSETGHGRRAFRRIHMELRE
KPDTDIKQFVIRELQKSCQCSAAKVRDGAFDFFPVLRWLPKYDLKKNILGDVMSGLIVGI
LLVPQSIAYSLLAGQEPIYGLYTSFFASIIYFLFGTSRHISVGIFGILCLMIGEVVDREL
HKACPDTDATSSSIAVFSSGCVVVNHTLDGLCDKSCYAIKIGSTVTFMAGVYQVAMGFFQ
VGFVSVYLSDALLSGFVTGASFTILTSQAKYLLGLSLPRSHGVGSVITTWIHIFRNIRNT
NICDLITSLLCLLVLVPSKELNEHFKDKLKAPIPVELIVVVAATLASHFGKLNGNYNSSI
AGHIPTGFMPPKAPDWSLIPNVAVDAIAISIIGFAITVSLSEMFAKKHGYTVKANQEMYA
IGFCNIIPSFFHCITTSAALAKTLVKESTGCQTQLSAIVTALVLLLVLLVIAPLFYSLQK
CVLGVITIVNLRGALLKFRDLPKMWRLSRMDTVIWFVTMLSSALLSTEIGLLVGVCFSMF
CVILRTQKPKNSLLGLEEESETFESISTYKNLRSKSGIKVFRFIAPLYYINKECFKSALY
KKALNPVLVKAAWKKAAKRKLKEEMVTFRGDPDEVSMQLSHDPLEVHTIVIDCSAIQFLD
TAGIHTLKEVRRDYEAVGIQVLLAQCNPSVRDSLARGEYCKKEEETLLFYSLSEAVAFAE
DSQNQKGVCVVNGLSLSGD
Human protein sequences
Amino acid sequence of galectin-2 from homo sapiens (SEQ ID NO: 14)
MTGELEVKNMDMKPGSTLKITGSIADGTDGFVINLGQGTDKLNLHFNPRFSESTIVCNSL
DGSNWGQEQREDHLCFSPGSEVKFTVTFESDKFKVKLPDGHELTFPNRLGHSHLSYLSVR
GGFNMSSFKLKE
Amino acid sequence of IgGFc-binding protein from homo sapiens (SEQ
ID NO: 15)
MGALWSWWILWAGATLLWGLTQEASVDLKNTGREEFLTAFLQNYQLAYSKAYPRLLISSL
SESPASVSILSQADNTSKKVTVRPGESVMVNISAKAEMIGSKIFQHAVVIHSDYAISVQA
LNAKPDTAELTLLRPIQALGTEYFVLTPPGTSARNVKEFAVVAGAAGASVSVTLKGSVTF
NGKFYPAGDVLRVTLQPYNVAQLQSSVDLSGSKVTASSPVAVLSGHSCAQKHTTCNHVVE
QLLPTSAWGTHYVVPTLASQSRYDLAFVVASQATKLTYNHGGITGSRGLQAGDVVEFEVR
PSWPLYLSANVGIQVLLFGTGAIRNEVTYDPYLVLIPDVAAYCPAYVVKSVPGCEGVALV
VAQTKAISGLTIDGHAVGAKLTWEAVPGSEFSYAEVELGTADMIHTAEATTNLGLLTFGL
AKAIGYATAADCGRTVLSPVEPSCEGMQCAAGQRCQVVGGKAGCVAESTAVCRAQGDPHY
TTFDGRRYDMMGTCSYTMVELCSEDDTLPAFSVEAKNEHRGSRRVSYVGLVTVRAYSHSV
SLTRGEVGFVLVDNQRSRLPVSLSEGRLRVYQSGPRAVVELVFGLVVTYDWDCQLALSLP
ARFQDQVCGLCGNYNGDPADDFLTPDGALAPDAVEFASSWKLDDGDYLCEDGCQNNCPAC
TPGQAQHYEGDRLCGMLTKLDGPFAVCHDTLDPRPFLEQCVYDLCVVGGERLSLCRGLSA
YAQACLELGISVGDWRSPANCPLSCPANSRYELCGPACPTSCNGAAAPSNCSGRPCVEGC
VCLPGFVASGGACVPASSCGCTFQGLQLAPGQEVWADELCQRRCTCNGATHQVTCRDKQS
CPAGERCSVQNGLLGCYPDRFGTCQGSGDPHYVSFDGRRFDFMGTCTYLLVGSCGQNAAL
PAFRVLVENEHRGSQTVSYTRAVRVEARGVKVAVRREYPGQVLVDDVLQYLPFQAADGQV
QVFRQGRDAVVRTDFGLTVTYDWNARVTAKVPSSYAEALCGLCGNFNGDPADDLALRGGG
QAANALAFGNSWQEETRPGCGATEPGDCPKLDSLVAQQLQSKNECGILADPKGPFRECHS
KLDPQGAVRDCVYDRCLLPGQSGPLCDALATYAAACQAAGATVHPWRSEELCPLSCPPHS
HYEACSYGCPLSCGDLPVPGGCGSECHEGCVCDEGFALSGESCLPLASCGCVHQGTYHPP
GQTFYPGPGCDSLCHCQEGGLVSCESSSCGPHEACQPSGGSLGCVAVGSSTCQASGDPHY
TTFDGRRFDFMGTCVYVLAQTCGTRPGLHRFAVLQENVAWGNGRVSVTRVITVQVANFTL
RLEQRQWKVTVNGVDMKLPVVLANGQIRASQHGSDVVIETDFGLRVAYDLVYYVRVTVPG
NYYQQMCGLCGNYNGDPKDDFQKPNGSQAGNANEFGNSWEEVVPDSPCLPPTPCPPGSED
CIPSHKCPPELEKKYQKEEFCGLLSSPTGPLSSCHKLVDPQGPLKDCIFDLCLGGGNLSI
LCSNIHAYVSACQAAGGHVEPWRTETFCPMECPPNSHYELCADTCSLGCSALSAPPQCQD
GCAEGCQCDSGFLYNGQACVPIQQCGCYHNGVYYEPEQTVLIDNCRQQCTCHAGKGMVCQ
EHSCKPGQVCQPSGGILSCVTKDPCHGVTCRPQETCKEQGGQGVCLPNYEATCWLWGDPH
YHSFDGRKFDFQGTCNYVLATTGCPGVSTQGLTPFTVTTKNQNRGNPAVSYVRVVTVAAL
GTNISIHKDEIGKVRVNGVLTALPVSVADGRISVTQGASKALLVADFGLQVSYDWNWRVD
VTLPSSYHGAVCGLCGNMDRNPNNDQVFPNGTLAPSIPIWGGSWRAPGWDPLCWDECRGS
CPTCPEDRLEQYEGPGFCGPLAPGTGGPFTTCHAHVPPESFFKGCVLDVCMGGGDRDILC
KALASYVAACQAAGVVIEDWRAQVGCEITCPENSHYEVCGSPCPASCPSPAPLTTPAVCE
GPCVEGCQCDAGFVLSADRCVPLNNGCGCWANGTYHEAGSEFWADGTCSQWCRCGPGGGS
LVCTPASCGLGEVCGLLPSGQHGCQPVSTAECQAWGDPHYVTLDGHRFNFQGTCEYLLSA
PCHGPPLGAENFTVTVANEHRGSQAVSYTRSVTLQIYNHSLTLSARWPRKLQVDGVFVTL
PFQLDSLLHAHLSGADVVVTTTSGLSLAFDGDSFVRLRVPAAYAGSLCGLCGNYNQDPAD
DLKAVGGKPAGWQVGGAQGCGECVSKPCPSPCTPEQQESFGGPDACGVISATDGPLAPCH
GLVPPAQYFQGCLLDACQVQGHPGGLCPAVATYVAACQAAGAQLREWRRPDFCPFQCPAH
SHYELCGDSCPGSCPSLSAPEGCESACREGCVCDAGFVLSGDTCVPVGQCGCLHDDRYYP
LGQTFYPGPGCDSLCRCREGGEVSCEPSSCGPHETCRPSGGSLGCVAVGSTTCQASGDPH
YTTFDGRRFDFMGTCVYVLAQTCGTRPGLHRFAVLQENVAWGNGRVSVTRVITVQVANFT
LRLEQRQWKVTVNGVDMKLPVVLANGQIRASQHGSDVVIETDFGLRVAYDLVYYVRVTVP
GNYYQLMCGLCGNYNGDPKDDFQKPNGSQAGNANEFGNSWEEVVPDSPCLPPPTCPPGSE
GCIPSEECPPELEKKYQKEEFCGLLSSPTGPLSSCHKLVDPQGPLKDCIFDLCLGGGNLS
ILCSNIHAYVSACQAAGGQVEPWRNETFCPMECPQNSHYELCADTCSLGCSALSAPLQCP
DGCAEGCQCDSGFLYNGQACVPIQQCGCYHNGAYYEPEQTVLIDNCRQQCTCHVGKVVVC
QEHSCKPGQVCQPSGGILSCVNKDPCHGVTCRPQETCKEQGGQGVCLPNYEATCWLWGDP
HYHSFDGRKFDFQGTCNYVLATTGCPGVSTQGLTPFTVTTKNQNRGNPAVSYVRVVTVAA
LGTNISIHKDEIGKVRVNGVLTALPVSVADGRISVTQGASKALLVADFGLQVSYDWNWRV
DVTLPSSYHGAVCGLCGNMDRNPNNDQVFPNGTLAPSIPIWGGSWRAPGWDPLCWDECRG
SCPTCPEDRLEQYEGPGFCGPLAPGTGGPFTTCHAHVPPESFFKGCVLDVCMGGGDRDIL
CKALASYVAACQAAGVVIEDWRAQVGCEITCPENSHYEVCGPPCPASCPSPAPLTTPAVC
EGPCVEGCQCDAGFVLSADRCVPLNNGCGCWANGTYHEAGSEFWADGTCSQWCRCGPGGG
SLVCTPASCGLGEVCGLLPSGQHGCQPVSTAECQAWGDPHYVTLDGHRFDFQGTCEYLLS
APCHGPPLGAENFTVTVANEHRGSQAVSYTRSVTLQIYNHSLTLSARWPRKLQVDGVFVT
LPFQLDSLLHAHLSGADVVVTTTSGLSLAFDGDSFVRLRVPAAYAGSLCGLCGNYNQDPA
DDLKAVGGKPAGWQVGGAQGCGECVSKPCPSPCTPEQQESFGGPDACGVISATDGPLAPC
HGLVPPAQYFQGCLLDACQVQGHPGGLCPAVATYVAACQAAGAQLREWRRPDFCPFQCPA
HSHYELCGDSCPGSCPSLSAPEGCESACREGCVCDAGFVLSGDTCVPVGQCGCLHDDRYY
PLGQTFYPGPGCDSLCRCREGGEVSCEPSSCGPHETCRPSGGSLGCVAVGSTTCQASGDP
HYTTFDGHRFDFMGTCVYVLAQTCGTRPGLHRFAVLQENVAWGNGRVSVTRVITVQVANF
TLRLEQRQWKVTVNGVDMKLPVVLANGQIRASQHGSDVVIETDFGLRVAYDLVYYVRVTV
PGNYYQLMCGLCGNYNGDPKDDFQKPNGSQAGNANEFGNSWEEVVPDSPCLPPPTCPPGS
AGCIPSDKCPPELEKKYQKEEFCGLLSSPTGPLSSCHKLVDPQGPLKDCIFDLCLGGGNL
SILCSNIHAYVSACQAAGGHVEPWRNETFCPMECPQNSHYELCADTCSLGCSALSAPLQC
PDGCAEGCQCDSGFLYNGQACVPIQQCGCYHNGVYYEPEQTVLIDNCRQQCTCHVGKVVV
CQEHSCKPGQVCQPSGGILSCVTKDPCHGVTCRPQETCKEQGGQGVCLPNYEATCWLWGD
PHYHSFDGRKFDFQGTCNYVLATTGCPGVSTQGLTPFTVTTKNQNRGNPAVSYVRVVTVA
ALGTNISIHKDEIGKVRVNGVLTALPVSVADGRISVAQGASKALLVADFGLQVSYDWNWR
VDVTLPSSYHGAVCGLCGNMDRNPNNDQVFPNGTLAPSIPIWGGSWRAPGWDPLCWDECR
GSCPTCPEDRLEQYEGPGFCGPLSSGTGGPFTTCHAHVPPESFFKGCVLDVCMGGGDRDI
LCKALASYVAACQAAGVVIEDWRAQVGCEITCPENSHYEVCGPPCPASCPSPAPLTTPAV
CEGPCVEGCQCDAGFVLSADRCVPLNNGCGCWANGTYHEAGSEFWADGTCSQWCRCGPGG
GSLVCTPASCGLGEVCGLLPSGQHGCQPVSTAECQAWGDPHYVTLDGHRFDFQGTCEYLL
SAPCHGPPLGAENFTVTVANEHRGSQAVSYTRSVTLQIYNHSLTLSARWPRKLQVDGVFV
ALPFQLDSLLHAHLSGADVVVTTTSGLSLAFDGDSFVRLRVPAAYAASLCGLCGNYNQDP
ADDLKAVGGKPAGWQVGGAQGCGECVSKPCPSPCTPEQQESFGGPDACGVISATDGPLAP
CHGLVPPAQYFQGCLLDACQVQGHPGGLCPAVATYVAACQAAGAQLGEWRRPDFCPLQCP
AHSHYELCGDSCPVSCPSLSAPEGCESACREGCVCDAGFVLSGDTCVPVGQCGCLHDGRY
YPLGEVFYPGPECERRCECGPGGHVTCQEGAACGPHEECRLEDGVQACHATGCGRCLANG
GIHYITLDGRVYDLHGSCSYVLAQVCHPKPGDEDFSIVLEKNAAGDLQRLLVTVAGQVVS
LAQGQQVTVDGEAVALPVAVGRVRVTAEGRNMVLQTTKGLRLLFDGDAHLLMSIPSPFRG
RLCGLCGNFNGNWSDDFVLPNGSAASSVETFGAAWRAPGSSKGCGEGCGPQGCPVCLAEE
TAPYESNEACGQLRNPQGPFATCQAVLSPSEYFRQCVYDLCAQKGDKAFLCRSLAAYTAA
CQAAGVAVKPWRTDSFCPLHCPAHSHYSICTRTCQGSCAALSGLTGCTTRCFEGCECDDR
FLLSQGVCIPVQDCGCTHNGRYLPVNSSLLTSDCSERCSCSSSSGLTCQAAGCPPGRVCE
VKAEARNCWATRGLCVLSVGANLTTFDGARGATTSPGVYELSSRCPGLQNTIPWYRVVAE
VQICHGKTEAVGQVHIFFQDGMVTLTPNKGVWVNGLRVDLPAEKLASVSVSRTPDGSLLV
RQKAGVQVWLGANGKVAVIVSNDHAGKLCGACGNFDGDQTNDWHDSQEKPAMEKWRAQDF
SPCYG
Amino acid sequence of galectin-4 from homo sapiens (SEQ ID NO: 16)
MAYVPAPGYQPTYNPTLPYYQPIPGGLNVGMSVYIQGVASEHMKRFFVNFVVGQDPGSDV
AFHFNPRFDGWDKVVFNTLQGGKWGSEERKRSMPFKKGAAFELVFIVLAEHYKVVVNGNP
FYEYGHRLPLQMVTHLQVDGDLQLQSINFIGGQPLRPQGPPMMPPYPGPGHCHQQLNSLP
TMEGPPTFNPPVPYFGRLQGGLTARRTIIIKGYVPPTGKSFAINFKVGSSGDIALHINPR
MGNGTVVRNSLLNGSWGSEEKKITHNPFGPGQFFDLSIRCGLDRFKVYANGQHLFDFAHR
LSAFQRVDTLEIQGDVTLSYVQI
Amino acid sequence of mucin-2 from homo sapiens (SEQ ID NO: 17)
MGLPLARLAAVCLALSLAGGSELQTEGRTRYHGRNVCSTWGNFHYKTFDGDVFRFPGLCD
YNFASDCRGSYKEFAVHLKRGPGQAEAPAGVESILLTIKDDTIYLTRHLAVLNGAVVSTP
HYSPGLLIEKSDAYTKVYSRAGLTLMWNREDALMLELDTKFRNHTCGLCGDYNGLQSYSE
FLSDGVLFSPLEFGNMQKINQPDVVCEDPEEEVAPASCSEHRAECERLLTAEAFADCQDL
VPLEPYLRACQQDRCRCPGGDTCVCSTVAEFSRQCSHAGGRPGNWRTATLCPKTCPGNLV
YLESGSPCMDTCSHLEVSSLCEEHRMDGCFCPEGTVYDDIGDSGCVPVSQCHCRLHGHLY
TPGQEITNDCEQCVCNAGRWVCKDLPCPGTCALEGGSHITTFDGKTYTFHGDCYYVLAKG
DHNDSYALLGELAPCGSTDKQTCLKTVVLLADKKKNAVVFKSDGSVLLNQLQVNLPHVTA
SFSVFRPSSYHIMVSMAIGVRLQVQLAPVMQLFVTLDQASQGQVQGLCGNFNGLEGDDFK
TASGLVEATGAGFANTWKAQSTCHDKLDWLDDPCSLNIESANYAEHWCSLLKKTETPFGR
CHSAVDPAEYYKRCKYDTCNCQNNEDCLCAALSSYARACTAKGVMLWGWREHVCNKDVGS
CPNSQVFLYNLTTCQQTCRSLSEADSHCLEGFAPVDGCGCPDHTFLDEKGRCVPLAKCSC
YHRGLYLEAGDVVVRQEERCVCRDGRLHCRQIRLIGQSCTAPKIHMDCSNLTALATSKPR
ALSCQTLAAGYYHTECVSGCVCPDGLMDDGRGGCVVEKECPCVHNNDLYSSGAKIKVDCN
TCTCKRGRWVCTQAVCHGTCSIYGSGHYITFDGKYYDFDGHCSYVAVQDYCGQNSSLGSF
SIITENVPCGTTGVTCSKAIKIFMGRTELKLEDKHRVVIQRDEGHHVAYTTREVGQYLVV
ESSTGIIVIWDKRTTVFIKLAPSYKGTVCGLCGNFDHRSNNDFTTRDHMVVSSELDFGNS
WKEAPTCPDVSTNPEPCSLNPHRRSWAEKQCSILKSSVFSICHSKVDPKPFYEACVHDSC
SCDTGGDCECFCSAVASYAQECTKEGACVFWRTPDLCPIFCDYYNPPHECEWHYEPCGNR
SFETCRTINGIHSNISVSYLEGCYPRCPKDRPIYEEDLKKCVTADKCGCYVEDTHYPPGA
SVPTEETCKSCVCTNSSQVVCRPEEGKILNQTQDGAFCYWEICGPNGTVEKHFNICSITT
RPSTLTTFTTITLPTTPTSFTTTTTTTTPTSSTVLSTTPKLCCLWSDWINEDHPSSGSDD
GDREPFDGVCGAPEDIECRSVKDPHLSLEQHGQKVQCDVSVGFICKNEDQFGNGPFGLCY
DYKIRVNCCWPMDKCITTPSPPTTTPSPPPTTTTTLPPTTTPSPPTTTTTTPPPTTTPSP
PITTTTTPLPTTTPSPPISTTTTPPPTTTPSPPTTTPSPPTTTPSPPTTTTTTPPPTTTP
SPPMTTPITPPASTTTLPPTTTPSPPTTTTTTPPPTTTPSPPTTTPITPPTSTTTLPPTT
TPSPPPTTTTTPPPTTTPSPPTTTTPSPPTITTTTPPPTTTPSPPTTTTTTPPPTTTPSP
PTTTPITPPTSTTTLPPTTTPSPPPTTTTTPPPTTTPSPPTTTTPSPPITTTTTPPPTTT
PSSPITTTPSPPTTTMTTPSPTTTPSSPITTTTTPSSTTTPSPPPTTMTTPSPTTTPSPP
TTTMTTLPPTTTSSPLTTTPLPPSITPPTFSPFSTTTPTTPCVPLCNWTGWLDSGKPNFH
KPGGDTELIGDVCGPGWAANISCRATMYPDVPIGQLGQTVVCDVSVGLICKNEDQKPGGV
IPMAFCLNYEINVQCCECVTQPTTMTTTTTENPTPPTTTPITTTTTVTPTPTPTGTQTPT
TTPITTTTTVTPTPTPTGTQTPTTTPITTTTTVTPTPTPTGTQTPTTTPITTTTTVTPTP
TPTGTQTPTTTPITTTTTVTPTPTPTGTQTPTTTPITTTTTVTPTPTPTGTQTPTTTPIT
TTTTVTPTPTPTGTQTPTTTPITTTTTVTPTPTPTGTQTPTTTPITTTTTVTPTPTPTGT
QTPTTTPITTTTTVTPTPTPTGTQTPTTTPITTTTTVTPTPTPTGTQTPTTTPITTTTTV
TPTPTPTGTQTPTTTPITTTTTVTPTPTPTGTQTPTTTPITTTTTVTPTPTPTGTQTPTT
TPITTTTTVTPTPTPTGTQTPTTTPITTTTTVTPTPTPTGTQTPTTTPITTTTTVTPTPT
PTGTQTPTTTPITTTTTVTPTPTPTGTQTPTTTPITTTTTVTPTPTPTGTQTPTTTPITT
TTTVTPTPTPTGTQTPTTTPITTTTTVTPTPTPTGTQTPTTTPITTTTTVTPTPTPTGTQ
TPTTTPITTTTTVTPTPTPTGTQTPTTTPITTTTTVTPTPTPTGTQTPTTTPITTTTTVT
PTPTPTGTQTPTTTPITTTTTVTPTPTPTGTQTPTTTPITTTTTVTPTPTPTGTQTPTTT
PITTTTTVTPTPTPTGTQTPTTTPITTTTTVTPTPTPTGTQTPTTTPITTTTTVTPTPTP
TGTQTPTTTPITTTTTVTPTPTPTGTQTPTTTPITTTTTVTPTPTPTGTQTPTTTPITTT
TTVTPTPTPTGTQTPTTTPITTTTTVTPTPTPTGTQTPTTTPITTTTTVTPTPTPTGTQT
PTTTPITTTTTVTPTPTPTGTQTPTTTPITTTTTVTPTPTPTGTQTPTTTPITTTTTVTP
TPTPTGTQTPTTTPITTTTTVTPTPTPTGTQTPTTTPITTTTTVTPTPTPTGTQTPTTTP
ITTTTTVTPTPTPTGTQTPTTTPITTTTTVTPTPTPTGTQTPTTTPITTTTTVTPTPTPT
GTQTPTTTPITTTTTVTPTPTPTGTQTPTTTPITTTTTVTPTPTPTGTQTPTTTPITTTT
TVTPTPTPTGTQTPTTTPITTTTTVTPTPTPTGTQTPTTTPITTTTTVTPTPTPTGTQTP
TTTPITTTTTVTPTPTPTGTQTPTTTPITTTTTVTPTPTPTGTQTPTTTPITTTTTVTPT
PTPTGTQTPTTTPITTTTTVTPTPTPTGTQTPTTTPITTTTTVTPTPTPTGTQTPTTTPI
TTTTTVTPTPTPTGTQTPTTTPITTTTTVTPTPTPTGTQTPTTTPITTTTTVTPTPTPTG
TQTPTTTPITTTTTVTPTPTPTGTQTPTTTPITTTTTVTPTPTPTGTQTPTTTPITTTTT
VTPTPTPTGTQTPTTTPITTTTTVTPTPTPTGTQTPTTTPITTTTTVTPTPTPTGTQTPT
TTPITTTTTVTPTPTPTGTQTPTTTPITTTTTVTPTPTPTGTQTPTTTPITTTTTVTPTP
TPTGTQTPTTTPITTTTTVTPTPTPTGTQTPTTTPITTTTTVTPTPTPTGTQTPTTTPIT
TTTTVTPTPTPTGTQTPTTTPITTTTTVTPTPTPTGTQTPTTTPITTTTTVTPTPTPTGT
QTPTTTPITTTTTVTPTPTPTGTQTPTTTPITTTTTVTPTPTPTGTQTPTTTPITTTTTV
TPTPTPTGTQTPTTTPITTTTTVTPTPTPTGTQTPTTTPITTTTTVTPTPTPTGTQTPTT
TPITTTTTVTPTPTPTGTQTPTTTPITTTTTVTPTPTPTGTQTPTTTPITTTTTVTPTPT
PTGTQTPTTTPITTTTTVTPTPTPTGTQTPTTTPITTTTTVTPTPTPTGTQTPTTTPITT
TTTVTPTPTPTGTQTPTTTPITTTTTVTPTPTPTGTQTPTTTPITTTTTVTPTPTPTGTQ
TPTTTPITTTTTVTPTPTPTGTQTPTTTPITTTTTVTPTPTPTGTQTPTTTPITTTTTVT
PTPTPTGTQTPTTTPITTTTTVTPTPTPTGTQTPTTTPITTTTTVTPTPTPTGTQTPTTT
PITTTTTVTPTPTPTGTQTPTTTPITTTTTVTPTPTPTGTQTPTTTPITTTTTVTPTPTP
TGTQTPTTTPITTTTTVTPTPTPTGTQTPTTTPITTTTTVTPTPTPTGTQTPTTTPITTT
TTVTPTPTPTGTQTPTTTPITTTTTVTPTPTPTGTQTPTTTPITTTTTVTPTPTPTGTQT
PTTTPITTTTTVTPTPTPTGTQTPTTTPITTTTTVTPTPTPTGTQTPTTTPITTTTTVTP
TPTPTGTQTPTTTPITTTTTVTPTPTPTGTQTPTTTPITTTTTVTPTPTPTGTQTGPPTH
TSTAPIAELTTSNPPPESSTPQTSRSTSSPLTESTTLLSTLPPAIEMTSTAPPSTPTAPT
TTSGGHTLSPPPSTTTSPPGTPTRGTTTGSSSAPTPSTVQTTTTSAWTPTPTPLSTPSII
RTTGLRPYPSSVLICCVLNDTYYAPGEEVYNGTYGDTCYFVNCSLSCTLEFYNWSCPSTP
SPTPTPSKSTPTPSKPSSTPSKPTPGTKPPECPDFDPPRQENETWWLCDCFMATCKYNNT
VEIVKVECEPPPMPTCSNGLQPVRVEDPDGCCWHWECDCYCTGWGDPHYVTFDGLYYSYQ
GNCTYVLVEEISPSVDNFGVYIDNYHCDPNDKVSCPRTLIVRHETQEVLIKTVHMMPMQV
QVQVNRQAVALPYKKYGLEVYQSGINYVVDIPELGVLVSYNGLSFSVRLPYHRFGNNTKG
QCGTCTNTTSDDCILPSGEIVSNCEAAADQWLVNDPSKPHCPHSSSTTKRPAVTVPGGGK
TTPHKDCTPSPLCQLIKDSLFAQCHALVPPQHYYDACVFDSCFMPGSSLECASLQAYAAL
CAQQNICLDWRNHTHGACLVECPSHREYQACGPAEEPTCKSSSSQQNNTVLVEGCFCPEG
TMNYAPGFDVCVKTCGCVGPDNVPREFGEHFEFDCKNCVCLEGGSGIICQPKRCSQKPVT
HCVEDGTYLATEVNPADTCCNITVCKCNTSLCKEKPSVCPLGFEVKSKMVPGRCCPFYWC
ESKGVCVHGNAEYQPGSPVYSSKCQDCVCTDKVDNNTLLNVIACTHVPCNTSCSPGFELM
EAPGECCKKCEQTHCIIKRPDNQHVILKPGDFKSDPKNNCTFFSCVKIHNQLISSVSNIT
CPNFDASICIPGSITFMPNGCCKTCTPRNETRVPCSTVPVTTEVSYAGCTKTVLMNHCSG
SCGTFVMYSAKAQALDHSCSCCKEEKTSQREVVLSCPNGGSLTHTYTHIESCQCQDTVCG
LPTGTSRRARRSPRHLGSG
Amino acid sequence of anterior gradient protein 2 homolog from homo
sapiens (SEQ ID NO: 18)
MEKIPVSAFLLLVALSYTLARDTTVKPGAKKDTKDSRPKLPQTLSRGWGDQLIWTQTYEE
ALYKSKTSNKPLMIIHHLDECPHSQALKKVFAENKEIQKLAEQFVLLNLVYETTDKHLSP
DGQYVPRIMFVDPSLTVRADITGRYSNRLYAYEPADTALLLDNMKKALKLLKTEL
Amino acid sequence of cadherin-17 from homo sapiens (SEQ ID NO: 19)
MILQAHLHSLCLLMLYLATGYGQEGKFSGPLKPMTFSIYEGQEPSQIIFQFKANPPAVTF
ELTGETDNIFVIEREGLLYYNRALDRETRSTHNLQVAALDANGIIVEGPVPITIKVKDIN
DNRPTFLQSKYEGSVRQNSRPGKPFLYVNATDLDDPATPNGQLYYQIVIQLPMINNVMYF
QINNKTGAISLTREGSQELNPAKNPSYNLVISVKDMGGQSENSFSDTTSVDIIVTENIWK
APKPVEMVENSTDPHPIKITQVRWNDPGAQYSLVDKEKLPRFPFSIDQEGDIYVTQPLDR
EEKDAYVFYAVAKDEYGKPLSYPLEIHVKVKDINDNPPTCPSPVTVFEVQENERLGNSIG
TLTAHDRDEENTANSFLNYRIVEQTPKLPMDGLFLIQTYAGMLQLAKQSLKKQDTPQYNL
TIEVSDKDFKTLCFVQINVIDINDQIPIFEKSDYGNLTLAEDTNIGSTILTIQATDADEP
FTGSSKILYHIIKGDSEGRLGVDTDPHTNTGYVIIKKPLDFETAAVSNIVFKAENPEPLV
FGVKYNASSFAKFTLIVTDVNEAPQFSQHVFQAKVSEDVAIGTKVGNVTAKDPEGLDISY
SLRGDTRGWLKIDHVTGEIFSVAPLDREAGSPYRVQVVATEVGGSSLSSVSEFHLILMDV
NDNPPRLAKDYTGLFFCHPLSAPGSLIFEATDDDQHLFRGPHFTFSLGSGSLQNDWEVSK
INGTHARLSTRHTEFEEREYVVLIRINDGGRPPLEGIVSLPVTFCSCVEGSCFRPAGHQT
GIPTVGMAVGILLTTLLVIGIILAVVFIRIKKDKGKDNVESAQASEVKPLRS
Amino acid sequence of zymogen granule membrane protein 16 from homo
sapiens (SEQ ID NO: 20)
MLTVALLALLCASASGNAIQARSSSYSGEYGGGGGKRFSHSGNQLDGPITALRVRVNTYY
IVGLQVRYGKVWSDYVGGRNGDLEEIFLHPGESVIQVSGKYKWYLKKLLFVTDKGRYLSF
GKDSGTSFNAVPLHPNTVLRFISGRSGSLIDAIGLHWDVYPSSCSRC
Amino acid sequence of metal transporter CNNM4 from homo sapiens (SEQ
ID NO: 21)
MAPVGGGGRPVGGPARGRLLLAAPVLLVLLWALGARGQGSPQQGTIVGMRLASCNKSCGT
NPDGIIFVSEGSTVNLRLYGYSLGNISSNLISFTEVDDAETLHKSTSCLELTKDLVVQQL
VNVSRGNTSGVLVVLTKFLRRSESMKLYALCTRAQPDGPWLKWTDKDSLLFMVEEPGRFL
PLWLHILLITVLLVLSGIFSGLNLGLMALDPMELRIVQNCGTEKERRYARKIEP1RRKGN
YLLCSLLLGNVLVNTSLTILLDNLIGSGLMAVASSTIGIVIFGEILPQALCSRHGLAVGA
NTILLTKFFMLLTFPLSFPISKLLDFFLGQEIRTVYNREKLMEMLKVTEPYNDLVKEELN
MIQGALELRTKTVEDIMTQLQDCFMIRSDAILDFNTMSEIMESGYTRIPVFEDEQSNIVD
ILYVKDLAFVDPDDCTPLKTITRFYNHPVHFVFHDTKLDAMLEEFKKGKSHLAIVQKVNN
EGEGDPFYEVLGLVTLEDVIEEIIKSEILDESDMYTDNRSRKRVSEKNKRDFSAFKDADN
ELKVKISPQLLLAAHRFLATEVSQFSPSLISEKILLRLLKYPDVIQELKFDEHNKYYARH
YLYTRNKPADYFILILQGKVEVEAGKENMKFETGAFSYYGTMALTSVPSDRSPAHPTPLS
RSASLSYPDRTDVSTAATLAGSSNQFGSSVLGQYISDFSVRALVDLQYIKITRQQYQNGL
LASRMENSPQFPIDGCTTHMENLAEKSELPVVDETTTLLNERNSLLHKASHENAI
Amino acid sequence of amiloride-sensitive amine oxidase [copper-
containing] from homo sapiens (SEQ ID NO: 22)
MPALGWAVAAILMLQTAMAEPSPGTLPRKAGVFSDLSNQELKAVHSFLWSKKELRLQPSS
TTTMAKNTVFLIEMLLPKKYHVLRFLDKGERHPVREARAVIFFGDQEHPNVTEFAVGPLP
GPCYMRALSPRPGYQSSWASRPISTAEYALLYHTLQEATKPLHQFFLNTTGFSFQDCHDR
CLAFTDVAPRGVASGQRRSWLIIQRYVEGYFLHPTGLELLVDHGSTDAGHWAVEQVWYNG
KFYGSPEELARKYADGEVDVVVLEDPLPGGKGHDSTEEPPLFSSHKPRGDFPSPIHVSGP
RLVQPHGPRFRLEGNAVLYGGWSFAFRLRSSSGLQVLNVHFGGERIAYEVSVQEAVALYG
GHTPAGMQTKYLDVGWGLGSVTHELAPGIDCPETATFLDTFHYYDADDPVHYPRALCLFE
MPTGVPLRRHFNSNFKGGFNFYAGLKGQVLVLRTTSTVYNYDYIWDFIFYPNGVMEAKMH
ATGYVHATFYTPEGLRHGTRLHTHLIGNIHTHLVHYRVDLDVAGTKNSFQTLQMKLENIT
NPWSPRHRVVQPTLEQTQYSWERQAAFRFKRKLPKYLLFTSPQENPWGHKRTYRLQIHSM
ADQVLPPGWQEEQAITWARYPLAVTKYRESELCSSSIYHQNDPWHPPVVFEQFLHNNENI
ENEDLVAWVTVGFLHIPHSEDIPNTATPGNSVGFLLRPFNFFPEDPSLASRDTVIVWPRD
NGPNYVQRWIPEDRDCSMPPPFSYNGTYRPV
Amino acid sequence of Hephaestin from homo sapiens (SEQ ID NO: 23)
MESGHLLWALLFMQSLWPQLTDGATRVYYLGIRDVQWNYAPKGRNVITNQPLDSDIVASS
FLKSDKNRIGGTYKKTIYKEYKDDSYTDEVAQPAWLGFLGPVLQAEVGDVILIHLKNFAT
RPYTIHPHGVFYEKDSEGSLYPDGSSGPLKADDSVPPGGSHIYNWTIPEGHAPTDADPAC
LTWIYHSHVDAPRDIATGLIGPLITCKRGALDGNSPPQRQDVDHDFFLLFSVVDENLSWH
LNENIATYCSDPASVDKEDETFQESNRMHAINGFVFGNLPELNMCAQKRVAWHLFGMGNE
IDVHTAFFHGQMLTTRGHHTDVANIFPATFVTAEMVPWEPGTWLISCQVNSHFRDGMQAL
YKVKSCSMAPPVDLLTGKVRQYFIEAHEIQWDYGPMGHDGSTGKNLREPGSISDKFFQKS
SSRIGGTYWKVRYEAFQDETFQEKMHLEEDRHLGILGPVIRAEVGDTIQVVFYNRASQPF
SMQPHGVFYEKDYEGTVYNDGSSYPGLVAKPFEKVTYRWTVPPHAGPTAQDPACLTWMYF
SAADPIRDTNSGLVGPLLVCRAGALGADGKQKGVDKEFFLLFTVLDENKSWYSNANQAAA
MLDFRLLSEDIEGFQDSNRMHAINGFLFSNLPRLDMCKGDTVAWHLLGLGTETDVHGVMF
QGNTVQLQGMRKGAAMLFPHTFVMAIMQPDNLGTFEIYCQAGSHREAGMRAIYNVSQCPG
HQATPRQRYQAARIYYIMAEEVEWDYCPDRSWEREWHNQSEKDSYGYIFLSNKDGLLGSR
YKKAVFREYTDGTFRIPRPRTGPEEHLGILGPLIKGEVGDILTVVFKNNASRPYSVHAHG
VLESTTVWPLAAEPGEVVTYQWNIPERSGPGPNDSACVSWIYYSAVDPIKDMYSGLVGPL
AlCQKGILEPHGGRSDMDREFALLFLIFDENKSWYLEENVATHGSQDPGSINLQDETFLE
SNKMHAINGKLYANLRGLTMYQGERVAWYMLAMGQDVDLHTIHFHAESFLYRNGENYRAD
VVDLFPGTFEVVEMVASNPGTWLMHCHVTDHVHAGMETLFTVFSRTEHLSPLTVITKETE
KAVPPRDIEEGNVKMLGMQIPIKNVEMLASVLVAISVTLLLVVLALGGVVWYQHRQRKLR
RNRRSILDDSFKLLSFKQ
Amino acid sequence of glutamine-fructose-6-phosphate aminotransferase
[isomerizing] 1 from homo sapiens (SEQ ID NO: 24)
MCGIFAYLNYHVPRTRREILETLIKGLQRLEYRGYDSAGVGFDGGNDKDWEANACKIQLI
KKKGKVKALDEEVHKQQDMDLDIEFDVHLGIAHTRWATHGEPSPVNSHPQRSDKNNEFIV
IHNGIITNYKDLKKFLESKGYDFESETDTETIAKLVKYMYDNRESQDTSFTTLVERVIQQ
LEGAFALVFKSVHFPGQAVGTRRGSPLLIGVRSEHKLSTDHIPILYRTARTQIGSKFTRW
GSQGERGKDKKGSCNLSRVDSTTCLFPVEEKAVEYYFASDASAVIEHTNRVIFLEDDDVA
AVVDGRLSIHRIKRTAGDHPGRAVQTLQMELQQIMKGNFSSFMQKEIFEQPESVVNTMRG
RVNFDDYTVNLGGLKDHIKEIQRCRRLILIACGTSYHAGVATRQVLEELTELPVMVELAS
DFLDRNTPVFRDDVCFFLSQSGETADTLMGLRYCKERGALTVGITNTVGSSISRETDCGV
HINAGPEIGVASTKAYTSQFVSLVMFALMMCDDRISMQERRKEIMLGLKRLPDLIKEVLS
MDDEIQKLATELYHQKSVLIMGRGYHYATCLEGALKIKEITYMHSEGILAGELKHGPLAL
VDKLMPVIMIIMRDHTYAKCQNALQQVVARQGRPVVICDKEDTETIKNTKRTIKVPHSVD
CLQGILSVIPLQLLAFHLAVLRGYDVDFPRNLAKSVTVE
Amino acid sequence of polymeric immunoglobulin receptor from homo
sapiens (SEQ ID NO: 25)
MLLFVLTCLLAVFPAISTKSPIFGPEEVNSVEGNSVSITCYYPPTSVNRHTRKYWCRQGA
RGGCITLISSEGYVSSKYAGRANLTNFPENGTFVVNIAQLSQDDSGRYKCGLGINSRGLS
FDVSLEVSQGPGLLNDTKVYTVDLGRTVTINCPFKTENAQKRKSLYKQIGLYPVLVIDSS
GYVNPNYTGRIRLDIQGTGQLLFSVVINQLRLSDAGQYLCQAGDDSNSNKKNADLQVLKP
EPELVYEDLRGSVTFHCALGPEVANVAKFLCRQSSGENCDVVVNTLGKRAPAFEGRILLN
PQDKDGSFSVVITGLRKEDAGRYLCGAHSDGQLQEGSPIQAWQLFVNEESTIPRSPTVVK
GVAGGSVAVLCPYNRKESKSIKYWCLWEGAQNGRCPLLVDSEGWVKAQYEGRLSLLEEPG
NGTFTVILNQLTSRDAGFYWCLTNGDTLWRTTVEIKIIEGEPNLKVPGNVTAVLGETLKV
PCHFPCKFSSYEKYWCKWNNTGCQALPSQDEGPSKAFVNCDENSRLVSLTLNLVTRADEG
WYWCGVKQGHFYGETAAVYVAVEERKAAGSRDVSLAKADAAPDEKVLDSGFREIENKAIQ
DPRLFAEEKAVADTRDQADGSRASVDSGSSEEQGGSSRALVSTLVPLGLVLAVGAVAVGV
ARARHRKNVDRVSIRSYRTDISMSDFENSREFGANDNMGASSITQETSLGGKEEFVATTE
STTETKEPKKAKRSSKEEAEMAYKDFLLQSSTVAAEAQDGPQEA
Amino acid sequence of sulfate transporter from homo sapiens (SEQ ID
NO: 26)
MSSESKEQHNVSPRDSAEGNDSYPSGIHLELQRESSTDFKQFETNDQCRPYHRILIERQE
KSDTNFKEFVIKKLQKNCQCSPAKAKNMILGFLPVLQWLPKYDLKKNILGDVMSGLIVGI
LLVPQSIAYSLLAGQEPVYGLYTSFFASIIYFLLGTSRHISVGIFGVLCLMIGETVDREL
QKAGYDNAHSAPSLGMVSNGSTLLNHTSDRICDKSCYAIMVGSTVTFIAGVYQVAMGFFQ
VGFVSVYLSDALLSGFVTGASFTILTSQAKYLLGLNLPRTNGVGSLITTWIHVFRNIHKT
NLCDLITSLLCLLVLLPTKELNEHFKSKLKAPIPIELVVVVAATLASHFGKLHENYNSSI
AGHIPTGFMPPKVPEWNLIPSVAVDAIAISIIGFAITVSLSEMFAKKHGYTVKANQEMYA
IGFCNIIPSFFHCFTTSAALAKTLVKESTGCHTQLSGVVTALVLLLVLLVIAPLFYSLQK
SVLGVITIVNLRGALRKFRDLPKMWSISRMDTVIWFVTMLSSALLSTEIGLLVGVCFSIF
CVILRTQKPKSSLLGLVEESEVFESVSAYKNLQIKPGIKIFRFVAPLYYINKECFKSALY
KQTVNPILIKVAWKKAAKRKIKEKVVTLGGIQDEMSVQLSHDPLELHTIVIDCSAIQFLD
TAGIHTLKEVRRDYEAIGIQVLLAQCNPTVRDSLTNGEYCKKEEENLLFYSVYEAMAFAE
VSKNQKGVCVPNGLSLSSD
Amino Acid sequence of cadherin-17 domain 1-2 from homo sapiens (SEQ ID
NO: 27)
PLKPMTFSIYEGQEPSQIIFQFKANPPAVTFELTGETDNIFVIEREGLLYYNRALDRETRSTHNLQVA
ALDANGIIVEGPVPITIKVKDINDNRPTFLQSKYEGSVRQNSRPGKPFLYVNATDLDDPATPNGQLYY
QIVIQLPMINNVMYFQINNKTGAISLTREGSQELNPAKNPSYNLVISVKDMGGQSENSFSDTTSVDII
VTENIWKAPKPV
Anti-human cadherin-17 antibody AV17-HCDR3 (SEQ ID NO: 28) amino acid
sequence
RPVMFDY
AV17-HCDR1 (SEQ ID NO: 29) amino acid sequence
SSYAMS
AV17-HCDR2 (SEQ ID NO: 30) amino acid sequence
AISGSGGSTYYADSVKG
AV17-LCDR1 (SEQ ID NO: 31) amino acid sequence
QGDSLRSYYAS
AV17-LCDR2 (SEQ ID NO: 32) amino acid sequence
GKNNRPS
AV17-LCDR3 (SEQ ID NO: 33) amino acid sequence
NSSLPSPQVV
AV17-VH (SEQ ID NO: 34) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKRPVMFDYWGQGTLVTVSS
AV17-VL (SEQ ID NO: 35) amino acid sequence
SSELTQDPAVSVALGQTVRITCQGDSLRSYYASWYQQKPGQAPVLVIYGKNNRPSGIPDRFSGSSSGN
TASLTITGAQAEDEADYYCNSSLPSPQVVVFGGGTKLTVLG
AV17-full-length scFv (SEQ ID NO: 36) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKRPVMFDYWGQGTLVTVSSGGGGSGGGGSGGGGSSELTQ
DPAVSVALGQTVRITCQGDSLRSYYASWYQQKPGQAPVLVIYGKNNRPSGIPDRFSGSSSGNTASLTI
TGAQAEDEADYYCNSSLPSPQVVVFGGGTKLTVLG
AV17 full-length scFv (SEQ ID NO: 37) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAACGTCCGGTGATGTTTGACTACTGGGGCCAGGGAACCCTGGTCACCG
TCTCGAGTGGTGGAGGCGGTTCAGGCGGAGGTGGCTCTGGCGGTGGCGGATCGTCTGAGCTGACTCAG
GACCCTGCTGTGTCTGTGGCCTTGGGACAGACAGTCAGGATCACATGCCAAGGAGACAGTCTCAGAAG
CTATTATGCAAGCTGGTACCAGCAGAAGCCAGGACAGGCCCCTGTACTTGTCATCTATGGTAAAAACA
ACCGGCCCTCAGGGATCCCAGACCGATTCTCTGGCTCCAGCTCAGGAAACACAGCTTCCTTGACCATC
ACTGGGGCTCAGGCGGAAGATGAGGCTGACTATTACTGTAACTCCTCTTTGCCCTCGCCTCAGGTTGT
GGTATTCGGCGGAGGGACCAAGCTGACCGTCCTAGGC
AV17-VH (SEQ ID NO: 38) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAACGTCCGGTGATGTTTGACTACTGGGGCCAGGGAACCCTGGTCACCG
TCTCGAGT
AV17-VL (SEQ ID NO: 39) nucleic acid sequence
TCGTCTGAGCTGACTCAGGACCCTGCTGTGTCTGTGGCCTTGGGACAGACAGTCAGGATCACATGCCA
AGGAGACAGTCTCAGAAGCTATTATGCAAGCTGGTACCAGCAGAAGCCAGGACAGGCCCCTGTACTTG
TCATCTATGGTAAAAACAACCGGCCCTCAGGGATCCCAGACCGATTCTCTGGCTCCAGCTCAGGAAAC
ACAGCTTCCTTGACCATCACTGGGGCTCAGGCGGAAGATGAGGCTGACTATTACTGTAACTCCTCTTT
GCCCTCGCCTCAGGTTGTGGTATTCGGCGGAGGGACCAAGCTGACCGTCCTAGGC
Linker (SEQ ID NO: 40) amino acid sequence
GGGGSGGGGSGGGG
Linker (SEQ ID NO: 41) nucleic acid sequence
GGTGGAGGCGGTTCAGGCGGAGGTGGCTCTGGCGGTGGCGGA
Anti-human cadherin-17 antibody AV17.2 HCDR3 (SEQ ID NO: 42) amino acid
sequence
RPHMFDY
AV17.2 HCDR1 (SEQ ID NO: 43) amino acid sequence
SSYAMS
AV17.2 HCDR2 (SEQ ID NO: 44) amino acid sequence
AISGSGGSTYYADSVKG
AV17.2 LCDR1 (SEQ ID NO: 45) amino acid sequence
QGDSLRSYYAS
AV17.2 LCDR2 (SEQ ID NO: 46) amino acid sequence
GKNNRPS
AV17.2 LCDR3 (SEQ ID NO: 47) amino acid sequence
NSSRPDSNGV
AV17.2 VH (SEQ ID NO: 48) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKRPHMFDYWGQGTLVTVSS
AV17.2 VL (SEQ ID NO: 49) amino acid sequence
SSELTQDPAVSVALGQTVRITCQGDSLRSYYASWYQQKPGQAPVLVIYGKNNRPSGIPDRFSGSSSGN
TASLTITGAQAEDEADYYCNSSRPDSNGVVFGGGTKLTVLG
AV17.2 full-length scFv (SEQ ID NO: 50) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKRPHMFDYWGQGTLVTVSSGGGGSGGGGSGGGGSSELTQ
DPAVSVALGQTVRITCQGDSLRSYYASWYQQKPGQAPVLVIYGKNNRPSGIPDRFSGSSSGNTASLTI
TGAQAEDEADYYCNSSRPDSNGVVFGGGTKLTVLG
AV17.2 full-length scFv (SEQ ID NO: 51) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAACGGCCGCATATGTTTGACTACTGGGGCCAGGGAACCCTGGTCACCG
TCTCGAGTGGTGGAGGCGGTTCAGGCGGAGGTGGCTCTGGCGGTGGCGGATCGTCTGAGCTGACTCAG
GACCCTGCTGTGTCTGTGGCCTTGGGACAGACAGTCAGGATCACATGCCAAGGAGACAGTCTCAGAAG
CTATTATGCAAGCTGGTACCAGCAGAAGCCAGGACAGGCCCCTGTACTTGTCATCTATGGTAAAAACA
ACCGGCCCTCAGGGATCCCAGACCGATTCTCTGGCTCCAGCTCAGGAAACACAGCTTCCTTGACCATC
ACTGGGGCTCAGGCGGAAGATGAGGCTGACTATTACTGTAACTCCTCTCGGCCCGATTCTAATGGTGT
GGTATTCGGCGGAGGGACCAAGCTGACCGTCCTAGGC
AV17.2 VH (SEQ ID NO: 52) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAACGGCCGCATATGTTTGACTACTGGGGCCAGGGAACCCTGGTCACCG
TCTCGAGT
AV17.2 VL (SEQ ID NO: 53) nucleic acid sequence
TCGTCTGAGCTGACTCAGGACCCTGCTGTGTCTGTGGCCTTGGGACAGACAGTCAGGATCACATGCCA
AGGAGACAGTCTCAGAAGCTATTATGCAAGCTGGTACCAGCAGAAGCCAGGACAGGCCCCTGTACTTG
TCATCTATGGTAAAAACAACCGGCCCTCAGGGATCCCAGACCGATTCTCTGGCTCCAGCTCAGGAAAC
ACAGCTTCCTTGACCATCACTGGGGCTCAGGCGGAAGATGAGGCTGACTATTACTGTAACTCCTCTCG
GCCCGATTCTAATGGTGTGGTATTCGGCGGAGGGACCAAGCTGACCGTCCTAGGC
Anti-anterior gradient protein 2 homolog antibody CPR02-HCDR3 (SEQ ID
NO: 54) amino acid sequence
GAGPTFDY
CPR02-HCDR1 (SEQ ID NO: 55) amino acid sequence
SSYAMS
CPR02-HCDR2 (SEQ ID NO: 56) amino acid sequence
AISGSGGSTYYADSVKG
CPR02 LCDR1 (SEQ ID NO: 57) amino acid sequence
QGDSLRSYYAS
CPR02 LCDR2 (SEQ ID NO: 58) amino acid sequence
GKNNRPS
CPR02 LCDR3 (SEQ ID NO: 59) amino acid sequence
NSSPPTRAVVV
CPR02-VH (SEQ ID NO: 60) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKGAGPTFDYWGQGTLVTVSS
CPR02-VL (SEQ ID NO: 61) amino acid sequence
SSELTQDPAVSVALGQTVRITCQGDSLRSYYASWYQQKPGQAPVLVIYGKNNRPSGIPDRFSGSSSGN
TASLTITGAQAEDEADYYCNSSPPTRAVVVFGGGTKLTVLG
CPR02 full-length scFv sequence (SEQ ID NO: 62) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKGAGPTFDYWGQGTLVTVSSGGGGSGGGGSGGGGSSELT
QDPAVSVALGQTVRITCQGDSLRSYYASWYQQKPGQAPVLVIYGKNNRPSGIPDRFSGSSSGNTASLT
ITGAQAEDEADYYCNSSPPTRAVVVFGGGTKLTVLG
CPR02-VH (SEQ ID NO: 63) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAGGTGCTGGGCCTACTTTTGACTACTGGGGCCAGGGAACCCTGGTCA
CCGTCTCGAGT
CPR02-VL (SEQ ID NO: 64) nucleic acid sequence
TCGTCTGAGCTGACTCAGGACCCTGCTGTGTCTGTGGCCTTGGGACAGACAGTCAGGATCACATGCCA
AGGAGACAGTCTCAGAAGCTATTATGCAAGCTGGTACCAGCAGAAGCCAGGACAGGCCCCTGTACTTG
TCATCTATGGTAAAAACAACCGGCCCTCAGGGATCCCAGACCGATTCTCTGGCTCCAGCTCAGGAAAC
ACAGCTTCCTTGACCATCACTGGGGCTCAGGCGGAAGATGAGGCTGACTATTACTGTAACTCCTCTCC
GCCCACGCGTGCTGTTGTGGTATTCGGCGGAGGGACCAAGCTGACCGTCCTAGGC
CPR02 Full scFv (SEQ ID NO: 65) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAGGTGCTGGGCCTACTTTTGACTACTGGGGCCAGGGAACCCTGGTCA
CCGTCTCGAGTGGTGGAGGCGGTTCAGGCGGAGGTGGCTCTGGCGGTGGCGGATCGTCTGAGCTGACT
CAGGACCCTGCTGTGTCTGTGGCCTTGGGACAGACAGTCAGGATCACATGCCAAGGAGACAGTCTCAG
AAGCTATTATGCAAGCTGGTACCAGCAGAAGCCAGGACAGGCCCCTGTACTTGTCATCTATGGTAAAA
ACAACCGGCCCTCAGGGATCCCAGACCGATTCTCTGGCTCCAGCTCAGGAAACACAGCTTCCTTGACC
ATCACTGGGGCTCAGGCGGAAGATGAGGCTGACTATTACTGTAACTCCTCTCCGCCCACGCGTGCTGT
TGTGGTATTCGGCGGAGGGACCAAGCTGACCGTCCTAGGC
Residues 41-175 of Agr2 from homo sapiens (SEQ ID NO: 66) amino acid
sequence
PQTLSRGWGDQLIWTQTYEEALYKSKTSNKPLMIIHHLDECPHSQALKKVFAENKEIQKLAEQFVLLN
LVYETTDKHLSPDGQYVPRIMFVDPSLTVRADITGRYSNRLYAYEPADTALLLDNMKKALKLLKTEL
Amino Acid sequence of Galectin-4 (Uniprot P56470, residues 1-323) from
homo sapiens (SEQ ID NO: 67)
MAYVPAPGYQPTYNPTLPYYQPIPGGLNVGMSVYIQGVASEHMKRFFVNFVVGQDPGSDVAFHFNPRF
DGWDKVVFNTLQGGKWGSEERKRSMPFKKGAAFELVFIVLAEHYKVVVNGNPFYEYGHRLPLQMVTHL
QVDGDLQLQSINFIGGQPLRPQGPPMMPPYPGPGHCHQQLNSLPTMEGPPTFNPPVPYFGRLQGGLTA
RRTIIIKGYVPPTGKSFAINFKVGSSGDIALHINPRMGNGTVVRNSLLNGSWGSEEKKITHNPFGPGQ
FFDLSIRCGLDRFKVYANGQHLFDFAHRLSAFQRVDTLEIQGDVTLSYVQI
Anti-galectin-4 scFv FF04-HCDR3 (SEQ ID NO: 68) amino acid sequence
MVWFELFDY
FF04-HCDR1 (SEQ ID NO: 69) amino acid sequence
SSYAMS
FF04-HCDR2 (SEQ ID NO: 70) amino acid sequence
AIKGSGGSTYYADSVKG
FF04-LCDR1 (SEQ ID NO: 71) amino acid sequence
RASQSVSSSYLA
FF04-LCDR2 (SEQ ID NO: 72) amino acid sequence
GASSRAT
FF04-LCDR3 (SEQ ID NO: 73) amino acid sequence
QQARGMPLT
FF04-VH (SEQ ID NO: 74) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKMVWFELFDYWGQGTLVTVSS
FF04-VL (SEQ ID NO: 75) amino acid sequence
EIVLTQSPGTLSLSPGERATLSCRASQSVSSSYLAWYQQKPGQAPRLLIYGASSRATGIPDRFSGSGS
GTDFTLTISRLEPEDFAVYYCQQARGMPLTFGQGTKVEIK
FF04 Full-length scFv (SEQ ID NO: 76) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKMVWFELFDYWGQGTLVTVSSGGGGSGGGGSGGGGEIVL
TQSPGTLSLSPGERATLSCRASQSVSSSYLAWYQQKPGQAPRLLIYGASSRATGIPDRFSGSGSGTDF
TLTISRLEPEDFAVYYCQQARGMPLTFGQGTKVEIK
FF04-VH (SEQ ID NO: 77) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAATGGTTTGGTTTGAGCTGTTTGACTACTGGGGCCAGGGAACCCTGG
TCACCGTCTCGAGT
FF04-VL (SEQ ID NO: 78) nucleic acid sequence
GAAATTGTGTTGACGCAGTCTCCAGGCACCCTGTCTTTGTCTCCAGGGGAAAGAGCCACCCTCTCCTG
CAGGGCCAGTCAGAGTGTTAGCAGCAGCTACTTAGCCTGGTACCAGCAGAAACCTGGCCAGGCTCCCA
GGCTCCTCATCTATGGTGCATCCAGCAGGGCCACTGGCATCCCAGACAGGTTCAGTGGCAGTGGGTCT
GGGACAGACTTCACTCTCACCATCAGCAGACTGGAGCCTGAAGATTTTGCAGTGTATTACTGTCAGCA
GGCGCGGGGTATGCCGCTTACGTTCGGCCAAGGGACCAAGGTGGAAATCAAA
FF04 Full-length scFv (SEQ ID NO: 79) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAATGGTTTGGTTTGAGCTGTTTGACTACTGGGGCCAGGGAACCCTGG
TCACCGTCTCGAGTGGTGGAGGCGGTTCAGGCGGAGGTGGCTCTGGCGGTGGCGGAGAAATTGTGTTG
ACGCAGTCTCCAGGCACCCTGTCTTTGTCTCCAGGGGAAAGAGCCACCCTCTCCTGCAGGGCCAGTCA
GAGTGTTAGCAGCAGCTACTTAGCCTGGTACCAGCAGAAACCTGGCCAGGCTCCCAGGCTCCTCATCT
ATGGTGCATCCAGCAGGGCCACTGGCATCCCAGACAGGTTCAGTGGCAGTGGGTCTGGGACAGACTTC
ACTCTCACCATCAGCAGACTGGAGCCTGAAGATTTTGCAGTGTATTACTGTCAGCAGGCGCGGGGTAT
GCCGCTTACGTTCGGCCAAGGGACCAAGGTGGAAATCAAA
Anti-galectin-4 scFv FF05-HCDR3 (SEQ ID NO: 80) amino acid sequence
TRRLKFDY
FF05-HCDR1 (SEQ ID NO: 81) amino acid sequence
SSYAMS
FF05-HCDR2 (SEQ ID NO: 82) amino acid sequence
AISGSGGSTYYADSVKG
FF05-LCDR1 (SEQ ID NO: 83) amino acid sequence
QGDSLRSYYAS
FF05-LCDR2 (SEQ ID NO: 84) amino acid sequence
GKNNRPS
FF05-LCDR3 (SEQ ID NO: 85) amino acid sequence
NSSAWNYPIVV
FF05-VH (SEQ ID NO: 86) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKTRRLKFDYWGQGTLVTVSS
FF05-VL (SEQ ID NO: 87) amino acid sequence
SSELTQDPAVSVALGQTVRITCQGDSLRSYYASWYQQKPGQAPVLVIYGKNNRPSGIPDRFSGSSSGN
TASLTITGAQAEDEADYYCNSSAWNYPIVVFGGGTKLTVLG
FF05-Full-length scFv (SEQ ID NO: 88) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKTRRLKFDYWGQGTLVTVSSGGGGSGGGGSGGGGSSELT
QDPAVSVALGQTVRITCQGDSLRSYYASWYQQKPGQAPVLVIYGKNNRPSGIPDRFSGSSSGNTASLT
ITGAQAEDEADYYCNSSAWNYPIVVFGGGTKLTVLG
FF05-VH (SEQ ID NO: 89) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAACGCGGCGTCTGAAGTTTGACTACTGGGGCCAGGGAACCCTGGTCA
CCGTCTCGAGT
FF05-VL (SEQ ID NO: 90) nucleic acid sequence
TCGTCTGAGCTGACTCAGGACCCTGCTGTGTCTGTGGCCTTGGGACAGACAGTCAGGATCACATGCCA
AGGAGACAGTCTCAGAAGCTATTATGCAAGCTGGTACCAGCAGAAGCCAGGACAGGCCCCTGTACTTG
TCATCTATGGTAAAAACAACCGGCCCTCAGGGATCCCAGACCGATTCTCTGGCTCCAGCTCAGGAAAC
ACAGCTTCCTTGACCATCACTGGGGCTCAGGCGGAAGATGAGGCTGACTATTACTGTAACTCCTCTGC
TTGGAATTATCCCATTGTGGTATTCGGCGGAGGGACCAAGCTGACCGTCCTAGGC
FF05-Full-length scFv (SEQ ID NO: 91) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAACGCGGCGTCTGAAGTTTGACTACTGGGGCCAGGGAACCCTGGTCA
CCGTCTCGAGTGGTGGAGGCGGTTCAGGCGGAGGTGGCTCTGGCGGTGGCGGATCGTCTGAGCTGACT
CAGGACCCTGCTGTGTCTGTGGCCTTGGGACAGACAGTCAGGATCACATGCCAAGGAGACAGTCTCAG
AAGCTATTATGCAAGCTGGTACCAGCAGAAGCCAGGACAGGCCCCTGTACTTGTCATCTATGGTAAAA
ACAACCGGCCCTCAGGGATCCCAGACCGATTCTCTGGCTCCAGCTCAGGAAACACAGCTTCCTTGACC
ATCACTGGGGCTCAGGCGGAAGATGAGGCTGACTATTACTGTAACTCCTCTGCTTGGAATTATCCCAT
TGTGGTATTCGGCGGAGGGACCAAGCTGACCGTCCTAGGC
Amino Acid sequence of galectin-2 from homo sapiens (SEQ ID NO: 92)
MTGELEVKNMDMKPGSTLKITGSIADGTDGFVINLGQGTDKLNLHFNPRFSESTIVCNSLDGSNWGQE
QREDHLCFSPGSEVKFTVTFESDKFKVKLPDGHELTFPNRLGHSHLSYLSVRGGFNMSSFKLKE
Anti-galectin-2 scFv FF06-HCDR3 (SEQ ID NO: 93) amino acid sequence
TRPFRVFDY
FF06-HCDR1 (SEQ ID NO: 94) amino acid sequence
SSYAMS
FF06-HCDR2 (SEQ ID NO: 95) amino acid sequence
AISGSGGSTYYADSVKG
FF06-LCDR1 (SEQ ID NO: 96) amino acid sequence
QGDSLRSYYAS
FF06-LCDR2 (SEQ ID NO: 97) amino acid sequence
GKNNRPS
FF06-LCDR3 (SEQ ID NO: 98) amino acid sequence
NSSRSRRPAVV
FF06-VH (SEQ ID NO: 99) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKTRPFRVFDYWGQGTLVTVSS
FF06-VL (SEQ ID NO: 100) amino acid sequence
SSELTQDPAVSVALGQTVRITCQGDSLRSYYASWYQQKPGQAPVLVIYGKNNRPSGIPDRFSGSSSGN
TASLTITGAQAEDEADYYCNSSRSRRPAVVFGGGTKLTVLG
FF06 Full-length scFv (SEQ ID NO: 101) amino acid sequence
EVQLLESGGGLVQPGGSLRLSCAASGFTFSSYAMSWVRQAPGKGLEWVSAISGSGGSTYYADSVKGRF
TISRDNSKNTLYLQMNSLRAEDTAVYYCAKTRPFRVFDYWGQGTLVTVSSGGGGSGGGGSGGGGSSEL
TQDPAVSVALGQTVRITCQGDSLRSYYASWYQQKPGQAPVLVIYGKNNRPSGIPDRFSGSSSGNTASL
TITGAQAEDEADYYCNSSRSRRPAVVFGGGTKLTVLG
FF06-VH (SEQ ID NO: 102) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAACTCGGCCGTTTAGGGTTTTTGACTACTGGGGCCAGGGAACCCTGG
TCACCGTCTCGAGT
FF06-VL (SEQ ID NO: 103) nucleic acid sequence
TCGTCTGAGCTGACTCAGGACCCTGCTGTGTCTGTGGCCTTGGGACAGACAGTCAGGATCACATGCCA
AGGAGACAGTCTCAGAAGCTATTATGCAAGCTGGTACCAGCAGAAGCCAGGACAGGCCCCTGTACTTG
TCATCTATGGTAAAAACAACCGGCCCTCAGGGATCCCAGACCGATTCTCTGGCTCCAGCTCAGGAAAC
ACAGCTTCCTTGACCATCACTGGGGCTCAGGCGGAAGATGAGGCTGACTATTACTGTAACTCCTCTCG
GTCGCGGCGTCCCGCTGTGGTATTCGGCGGAGGGACCAAGCTGACCGTCCTAGGC
FF06 Full-length scFv (SEQ ID NO: 104) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAGTGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAACTCGGCCGTTTAGGGTTTTTGACTACTGGGGCCAGGGAACCCTGG
TCACCGTCTCGAGTGGTGGAGGCGGTTCAGGCGGAGGTGGCTCTGGCGGTGGCGGATCGTCTGAGCTG
ACTCAGGACCCTGCTGTGTCTGTGGCCTTGGGACAGACAGTCAGGATCACATGCCAAGGAGACAGTCT
CAGAAGCTATTATGCAAGCTGGTACCAGCAGAAGCCAGGACAGGCCCCTGTACTTGTCATCTATGGTA
AAAACAACCGGCCCTCAGGGATCCCAGACCGATTCTCTGGCTCCAGCTCAGGAAACACAGCTTCCTTG
ACCATCACTGGGGCTCAGGCGGAAGATGAGGCTGACTATTACTGTAACTCCTCTCGGTCGCGGCGTCC
CGCTGTGGTATTCGGCGGAGGGACCAAGCTGACCGTCCTAGGC
Anti-human cadherin-17 antibody AV17.3 HCDR3 (SEQ ID NO: 105) amino acid
sequence
MSIRGFDY
AV17.3 HCDR1 (SEQ ID NO: 106) amino acid sequence
SSYAMS
AV17.3 HCDR2 (SEQ ID NO: 107) amino acid sequence
AIKGSGGSTYYADSVKG
AV17.3 LCDR1 (SEQ ID NO: 108) amino acid sequence
QGDSLRSYYAS
AV17.3 LCDR2 (SEQ ID NO: 109) amino acid sequence
GKNNRPS
AV17.3 LCDR3 (SEQ ID NO: 110) amino acid sequence
NSSRGVRPLV
AV17.3 VH (SEQ ID NO: 111) amino acid sequence
EVQLLESGGGLVQPGGSLRLSCAASGFTFSSYAMSWVRQAPGKGLEWVSAIKGSGGSTYYADSVKGRF
TISRDNSKNTLYLQMNSLRAEDTAVYYCAKMSIRGFDYWGQGTLVTVSS
AV17.3 VL (SEQ ID NO: 112) amino acid sequence
SSELTQDPAVSVALGQTVRITCQGDSLRSYYASWYQQKPGQAPVLVIYGKNNRPSGIPDRFSGSSSGN
TASLTITGAQAEDEADYYCNSSRGVRPLVVFGGGTKLTVLG
AV17.3 full-length scFv (SEQ ID NO: 113) amino acid sequence
EVQLLESGGGLVQPGGSLRLSCAASGFTFSSYAMSWVRQAPGKGLEWVSAIKGSGGSTYYADSVKGRF
TISRDNSKNTLYLQMNSLRAEDTAVYYCAKMSIRGFDYWGQGTLVTVSSGGGGSGGGGSGGGGSSELT
QDPAVSVALGQTVRITCQGDSLRSYYASWYQQKPGQAPVLVIYGKNNRPSGIPDRFSGSSSGNTASLT
ITGAQAEDEADYYCNSSRGVRPLVVFGGGTKLTVLG
AV17.3 full-length scFv (SEQ ID NO: 114) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAAGGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAATGTCGATTAGGGGTTTTGACTACTGGGGCCAGGGAACCCTGGTCA
CCGTCTCGAGTGGTGGAGGCGGTTCAGGCGGAGGTGGCTCTGGCGGTGGCGGATCGTCTGAGCTGACT
CAGGACCCTGCTGTGTCTGTGGCCTTGGGACAGACAGTCAGGATCACATGCCAAGGAGACAGTCTCAG
AAGCTATTATGCAAGCTGGTACCAGCAGAAGCCAGGACAGGCCCCTGTACTTGTCATCTATGGTAAAA
ACAACCGGCCCTCAGGGATCCCAGACCGATTCTCTGGCTCCAGCTCAGGAAACACAGCTTCCTTGACC
ATCACTGGGGCTCAGGCGGAAGATGAGGCTGACTATTACTGTAACTCCTCTAGGGGGGTTCGTCCCTT
GGTGGTATTCGGCGGAGGGACCAAGCTGACCGTCCTAGGC
AV17.3 VH (SEQ ID NO: 115) nucleic acid sequence
GAGGTGCAGCTGTTGGAGTCTGGGGGAGGCTTGGTACAGCCTGGGGGGTCCCTGAGACTCTCCTGTGC
AGCCTCTGGATTCACCTTTAGCAGCTATGCCATGAGCTGGGTCCGCCAGGCTCCAGGGAAGGGGCTGG
AGTGGGTCTCAGCTATTAAGGGTAGTGGTGGTAGCACATACTACGCAGACTCCGTGAAGGGCCGGTTC
ACCATCTCCAGAGACAATTCCAAGAACACGCTGTATCTGCAAATGAACAGCCTGAGAGCCGAAGACAC
GGCCGTATATTACTGTGCGAAAATGTCGATTAGGGGTTTTGACTACTGGGGCCAGGGAACCCTGGTCA
CCGTCTCGAGT
AV17.3 VL (SEQ ID NO: 116) nucleic acid sequence
TCGTCTGAGCTGACTCAGGACCCTGCTGTGTCTGTGGCCTTGGGACAGACAGTCAGGATCACATGCCA
AGGAGACAGTCTCAGAAGCTATTATGCAAGCTGGTACCAGCAGAAGCCAGGACAGGCCCCTGTACTTG
TCATCTATGGTAAAAACAACCGGCCCTCAGGGATCCCAGACCGATTCTCTGGCTCCAGCTCAGGAAAC
ACAGCTTCCTTGACCATCACTGGGGCTCAGGCGGAAGATGAGGCTGACTATTACTGTAACTCCTCTAG
GGGGGTTCGTCCCTTGGTGGTATTCGGCGGAGGGACCAAGCTGACCGTCCTAGGC

### REFERENCES

All documents mentioned in this specification are incorporated herein by reference in their entirety.
1. Hart, P. H., et al., Potential antiinflammatory effects of interleukin 4: suppression of human monocyte tumor necrosis factor alpha, interleukin 1, and prostaglandin E2. Proc Natl Acad Sci USA, 1989. 86(10): p. 3803-7.
2. Fenton, M. J., J. A. Buras, and R. P. Donnelly, IL-4 reciprocally regulates IL-1 and IL-1 receptor antagonist expression in human monocytes. J Immunol, 1992. 149(4): p. 1283-8.
3. Colotta, F., et al., Interleukin-1 type II receptor: a decoy target for IL-1 that is regulated by IL-4. Science, 1993. 261(5120): p. 472-5.
4. Cawston, T. E., et al., Interleukin-4 blocks the release of collagen fragments from bovine nasal cartilage treated with cytokines. Biochim Biophys Acta, 1996. 1314(3): p. 226-32.
5. Lacraz, S., et al., Suppression of metalloproteinase biosynthesis in human alveolar macrophages by interleukin-4. J Clin Invest, 1992. 90(2): p. 382-8.
6. Miossec, P., et al., Interleukin-4 inhibits bone resorption through an effect on osteoclasts and proinflammatory cytokines in an ex vivo model of bone resorption in rheumatoid arthritis. Arthritis Rheum, 1994. 37(12): p. 1715-22.
7. Gordon, S., Alternative activation of macrophages. Nat Rev Immunol, 2003. 3(1): p. 23-35.
8. Goerdt, S., et al., Alternative versus classical activation of macrophages. Pathobiology, 1999. 67(5-6): p. 222-6.
9. Ghoreschi, K., et al., Interleukin-4 therapy of psoriasis induces Th2 responses and improves human autoimmune disease. Nat Med, 2003. 9(1): p. 40-6.
10. Lubberts, E., et al., Adenoviral vector-mediated overexpression of IL-4 in the knee joint of mice with collagen-induced arthritis prevents cartilage destruction. J Immunol, 1999. 163(8): p. 4546-56.
11. Asadullah, K., W. Sterry, and H. D. Volk, Interleukin-10 therapy-review of a new approach. Pharmacol Rev, 2003. 55(2): p. 241-69.
12. de Waal Malefyt, R., et al., Interleukin 10 (IL-10) inhibits cytokine synthesis by human monocytes: an autoregulatory role of IL-10 produced by monocytes. J Exp Med, 1991. 174(5): p. 1209-20.
13. de Waal Malefyt, R., et al., Interleukin 10 (IL-10) and viral IL-10 strongly reduce antigen-specific human T cell proliferation by diminishing the antigen-presenting capacity of monocytes via downregulation of class II major histocompatibility complex expression. J Exp Med, 1991. 174(4): p. 915-24.
14. Fiorentino, D. F., et al., IL-10 inhibits cytokine production by activated macrophages. J Immunol, 1991. 147(11): p. 3815-22.
15. Fiorentino, D. F., et al., IL-10 acts on the antigen-presenting cell to inhibit cytokine production by Th1 cells. J Immunol, 1991. 146(10): p. 3444-51.
16. Romagnani, S., Biology of human TH1 and TH2 cells. J Clin Immunol, 1995. 15(3): p. 121-9.
17. Jenkins, J. K., M. Malyak, and W. P. Arend, The effects of interleukin-10 on interleukin-1 receptor antagonist and interleukin-1 beta production in human monocytes and neutrophils. Lymphokine Cytokine Res, 1994. 13(1): p. 47-54.
18. Hart, P. H., et al., Regulation of surface and soluble TNF receptor expression on human monocytes and synovial fluid macrophages by IL-4 and IL-10. J Immunol, 1996. 157(8): p. 3672-80.
19. Joyce, D. A., et al., Two inhibitors of pro-inflammatory cytokine release, interleukin-10 and interleukin-4, have contrasting effects on release of soluble p75 tumor necrosis factor receptor by cultured monocytes. Eur J Immunol, 1994. 24(11): p. 2699-705.
20. Moore, K. W., et al., Interleukin-10 and the interleukin-10 receptor. Annu Rev Immunol, 2001. 19: p. 683-765.
21. Ling, P., et al., Human IL-12 p40 homodimer binds to the IL-12 receptor but does not mediate biologic activity. J Immunol, 1995. 154(1): p. 116-27.
22. Gately, M. K., et al., Interleukin-12 antagonist activity of mouse interleukin-12 p40 homodimer in vitro and in vivo. Ann N Y Acad Sci, 1996. 795: p. 1-12.
23. Gillessen, S., et al., Mouse interleukin-12 (IL-12) p40 homodimer: a potent IL-12 antagonist. Eur J Immunol, 1995. 25(1): p. 200-6.
24. Shimozato, O., et al., The secreted form of the p40 subunit of interleukin (IL)-12 inhibits IL-23 functions and abrogates IL-23-mediated antitumour effects. Immunology, 2006. 117(1): p. 22-8.
25. Kim, D. J., et al., Delivery of IL-12p40 ameliorates DSS-induced colitis by suppressing IL-17A expression and inflammation in the intestinal mucosa. Clin Immunol, 2012. 144(3): p. 190-9.
26. Li, L. J., et al., Role of interleukin-22 in inflammatory bowel disease. World J Gastroenterol, 2014. 20(48): p. 18177-88.
27. Zenewicz, L. A. and R. A. Flavell, Recent advances in IL-22 biology. Int Immunol, 2011. 23(3): p. 159-63.
28. Sugimoto, K., et al., IL-22 ameliorates intestinal inflammation in a mouse model of ulcerative colitis. J Clin Invest, 2008. 118(2): p. 534-44.
29. Aparicio-Siegmund, S. and C. Garbers, The biology of interleukin-27 reveals unique pro- and anti-inflammatory functions in immunity. Cytokine Growth Factor Rev, 2015. 26(5): p. 579-86.
30. Sasaoka, T., et al., Treatment with IL-27 attenuates experimental colitis through the suppression of the development of IL-17-producing T helper cells. American journal of physiology. Gastrointestinal and liver physiology, 2011. 300(4): p. G568-76.
31. Vignali, D. A. and V. K. Kuchroo, IL-12 family cytokines: immunological playmakers. Nature immunology, 2012. 13(8): p. 722-8.
32. Collison, L. W. and D. A. Vignali, Interleukin-35: odd one out or part of the family? Immunological reviews, 2008. 226: p. 248-62.
33. Collison, L. W., et al., The inhibitory cytokine IL-35 contributes to regulatory T-cell function. Nature, 2007. 450(7169): p. 566-9.
34. Wirtz, S., et al., Interleukin-35 mediates mucosal immune responses that protect against T-cell-dependent colitis. Gastroenterology, 2011. 141(5): p. 1875-86.
35. Gasson, J. C., Molecular physiology of granulocyte-macrophage colony-stimulating factor. Blood, 1991. 77(6): p. 1131-45.
36. Hamilton, J. A., Colony-stimulating factors in inflammation and autoimmunity. Nat Rev Immunol, 2008. 8(7): p. 533-44.
37. Walsh, G., Biopharmaceutical benchmarks 2014. Nat Biotechnol, 2014. 32(10): p. 992-1000.
38. Korzenik, J. R., et al., Sargramostim for active Crohn's disease. N Engl J Med, 2005. 352(21): p. 2193-201.
39. Grobeta, P., et al., IL-33 attenuates development and perpetuation of chronic intestinal inflammation. Inflamm Bowel Dis, 2012. 18(10): p. 1900-9.
40. Pastorelli, L., et al., The role of IL-33 in gut mucosal inflammation. Mediators Inflamm, 2013. 2013: p. 608187.
41. Molofsky, A. B., A. K. Savage, and R. M. Locksley, Interleukin-33 in Tissue Homeostasis, Injury, and Inflammation. Immunity, 2015. 42(6): p. 1005-19.
42. Oboki, K., et al., IL-33 is a crucial amplifier of innate rather than acquired immunity. Proc Natl Acad Sci USA, 2010. 107(43): p. 18581-6.
43. Sedhom, M. A., et al., Neutralisation of the interleukin-33/ST2 pathway ameliorates experimental colitis through enhancement of mucosal healing in mice. Gut, 2013. 62(12): p. 1714-23.
44. Lopetuso, L. R., S. Chowdhry, and T. T. Pizarro, Opposing Functions of Classic and Novel IL-1 Family Members in Gut Health and Disease. Front Immunol, 2013. 4: p. 181.

1-10. (canceled)

11. An antibody molecule, or fragment thereof, which binds cadherin-17, wherein the antibody molecule, or fragment, comprises a VH domain comprising a framework and a set of complementarity determining regions HCDR1, HCDR2 and HCDR3, and a VL domain comprising a framework and a set of complementarity determining regions LCDR1, LCDR2 and LCDR3, wherein:
HCDR3 has the amino acid sequence set forth in SEQ ID NO: 28, or the amino acid sequence set forth in SEQ ID NO: 28 with three or fewer amino acid substitutions.

12. The antibody molecule, or fragment, according to claim 11, wherein LCDR3 has the amino acid sequence set forth in SEQ ID NO: 33, or the amino acid sequence set forth in SEQ ID NO: 33 with three or fewer amino acid substitutions.

13. The antibody molecule, or fragment, according to claim 11, wherein
HCDR1 has the amino acid sequence set forth in SEQ ID NO: 29, or the amino acid sequence set forth in SEQ ID NO: 29 with three or fewer amino acid substitutions;
HCDR2 has the amino acid sequence set forth in SEQ ID NO: 30, or the amino acid sequence set forth in SEQ ID NO: 30 with three or fewer amino acid substitutions;
LCDR1 has the amino acid sequence set forth in SEQ ID NO: 31, or the amino acid sequence set forth in SEQ ID NO: 31 with three or fewer amino acid substitutions; and/or
LCDR2 has the amino acid sequence set forth in SEQ ID NO: 32, or the amino acid sequence set forth in SEQ ID NO: 32 with three or fewer amino acid substitutions.

14. The antibody molecule, or fragment, according to claim 13, wherein the VH domain comprises or consist of the amino acid sequence set forth in SEQ ID NO: 34, and/or the VL domain comprises or consist of the amino acid sequence set forth in SEQ ID NO: 35.

15. The antibody molecule, or fragment, according to claim 14, wherein the antibody molecule, or fragment, is an scFv and comprises the amino acid sequence set forth in SEQ ID NO: 36.

16. An antibody molecule, or fragment thereof, which binds cadherin-17, wherein the antibody molecule, or fragment, comprises a VH domain comprising a framework and a set of complementarity determining regions HCDR1, HCDR2 and HCDR3, and a VL domain comprising a framework and a set of complementarity determining regions LCDR1, LCDR2 and LCDR3, wherein:
HCDR3 has the amino acid sequence set forth in SEQ ID NO: 42, or the amino acid sequence set forth in SEQ ID NO: 42 with three or fewer amino acid substitutions.

17. The antibody molecule, or fragment, according to claim 16, wherein LCDR3 has the amino acid sequence set forth in SEQ ID NO: 47, or the amino acid sequence set forth in SEQ ID NO: 47 with three or fewer amino acid substitutions.

18. The antibody molecule, or fragment, according to claim 16, wherein
HCDR1 has the amino acid sequence set forth in SEQ ID NO: 43, or the amino acid sequence set forth in SEQ ID NO: 43 with three or fewer amino acid substitutions;
HCDR2 has the amino acid sequence set forth in SEQ ID NO: 44, or the amino acid sequence set forth in SEQ ID NO: 44 with three or fewer amino acid substitutions;
LCDR1 has the amino acid sequence set forth in SEQ ID NO: 45, or the amino acid sequence set forth in SEQ ID NO: 45 with three or fewer amino acid substitutions; and/or
LCDR2 has the amino acid sequence set forth in SEQ ID NO: 46, or the amino acid sequence set forth in SEQ ID NO: 46 with three or fewer amino acid substitutions.

19. The antibody molecule, or fragment, according to claim 18, wherein the VH domain comprises or consist of the amino acid sequence set forth in SEQ ID NO: 48, and/or the VL domain comprises or consist of the amino acid sequence set forth in SEQ ID NO: 49.

20. The antibody molecule, or fragment, according to claim 19, wherein the antibody molecule, or fragment, is an scFv and comprises the amino acid sequence set forth in SEQ ID NO: 50.

21-49. (canceled)

50. The antibody molecule, or fragment, according to claim 12, wherein
HCDR1 has the amino acid sequence set forth in SEQ ID NO: 29, or the amino acid sequence set forth in SEQ ID NO: 29 with three or fewer amino acid substitutions;
HCDR2 has the amino acid sequence set forth in SEQ ID NO: 30, or the amino acid sequence set forth in SEQ ID NO: 30 with three or fewer amino acid substitutions;
LCDR1 has the amino acid sequence set forth in SEQ ID NO: 31, or the amino acid sequence set forth in SEQ ID NO: 31 with three or fewer amino acid substitutions; and/or
LCDR2 has the amino acid sequence set forth in SEQ ID NO: 32, or the amino acid sequence set forth in SEQ ID NO: 32 with three or fewer amino acid substitutions.

51. The antibody molecule, or fragment, according to claim 17, wherein
HCDR1 has the amino acid sequence set forth in SEQ ID NO: 43, or the amino acid sequence set forth in SEQ ID NO: 43 with three or fewer amino acid substitutions;
HCDR2 has the amino acid sequence set forth in SEQ ID NO: 44, or the amino acid sequence set forth in SEQ ID NO: 44 with three or fewer amino acid substitutions;
LCDR1 has the amino acid sequence set forth in SEQ ID NO: 45, or the amino acid sequence set forth in SEQ ID NO: 45 with three or fewer amino acid substitutions; and/or
LCDR2 has the amino acid sequence set forth in SEQ ID NO: 46, or the amino acid sequence set forth in SEQ ID NO: 46 with three or fewer amino acid substitutions.
